SEQUENCE LISTING (243 sequences)

SEQ ID NO: 1 - protein, 7 aa, Artificial Sequence
  Description of Artificial Sequence Synthetic peptide
XXXXXXX

SEQ ID NO: 2 - protein, 9 aa, Artificial Sequence
  Description of Artificial Sequence Synthetic peptide
XGXXXXXWX

SEQ ID NO: 3 - protein, 9 aa, Artificial Sequence
  Description of Artificial Sequence Synthetic peptide
XGPTLRXWX

SEQ ID NO: 4 - protein, 18 aa, Artificial Sequence
  Description of Artificial Sequence Synthetic peptide
GGCADGPTLREWISFCGG

SEQ ID NO: 5 - protein, 19 aa, Artificial Sequence
  Description of Artificial Sequence Synthetic peptide
GNADGPTLRQWLEGRRPKN

SEQ ID NO: 6 - protein, 19 aa, Artificial Sequence
  Description of Artificial Sequence Synthetic peptide
GGCADGPTLREWISFCGGK

SEQ ID NO: 7 - protein, 18 aa, Artificial Sequence
  Description of Artificial Sequence Synthetic peptide
TIKGPTLRQWLKSREHTS

SEQ ID NO: 8 - protein, 18 aa, Artificial Sequence
  Description of Artificial Sequence Synthetic peptide
SIEGPTLREWLTSRTPHS

SEQ ID NO: 9 - protein, 19 aa, Artificial Sequence
  Description of Artificial Sequence Synthetic peptide
LAIEGPTLRQWLHGNGRDT

SEQ ID NO: 10 - protein, 14 aa, Artificial Sequence
  Description of Artificial Sequence Synthetic peptide
CADGPTLREWISFC

SEQ ID NO: 11 - protein, 14 aa, Artificial Sequence
  Description of Artificial Sequence Synthetic peptide
IEGPTLRQWLAARA

SEQ ID NO: 12 - protein, 10 aa, Artificial Sequence
  Description of Artificial Sequence Synthetic peptide
XXGXXXXXWX

SEQ ID NO: 13 - protein, 7 aa, Artificial Sequence
  Description of Artificial Sequence Synthetic peptide
XXXXXXX

SEQ ID NO: 14 - protein, 9 aa, Artificial Sequence
  Description of Artificial Sequence Synthetic peptide
XWXXXXXGX

SEQ ID NO: 15 - protein, 9 aa, Artificial Sequence
  Description of Artificial Sequence Synthetic peptide
XWXRLTPGX

SEQ ID NO: 16 - protein, 10 aa, Artificial Sequence
  Description of Artificial Sequence Synthetic peptide
XWXXXXXGXX

SEQ ID NO: 17 - protein, 18 aa, Artificial Sequence
  Description of Artificial Sequence Synthetic peptide
GGCFSIWERLTPGDACGG

SEQ ID NO: 18 - protein, 19 aa, Artificial Sequence
  Description of Artificial Sequence Synthetic peptide
NKPRRGELWQRLTPGDANG

SEQ ID NO: 19 - protein, 19 aa, Artificial Sequence
  Description of Artificial Sequence Synthetic peptide
KGGCFSIWERLTPGDACGG

SEQ ID NO: 20 - protein, 18 aa, Artificial Sequence
  Description of Artificial Sequence Synthetic peptide
STHERSKLWQRLTPGKIT

SEQ ID NO: 21 - protein, 18 aa, Artificial Sequence
  Description of Artificial Sequence Synthetic peptide
SHPTRSTLWERLTPGEIS

SEQ ID NO: 22 - protein, 19 aa, Artificial Sequence
  Description of Artificial Sequence Synthetic peptide
TDRGNGHLWQRLTPGEIAL

SEQ ID NO: 23 - protein, 14 aa, Artificial Sequence
  Description of Artificial Sequence Synthetic peptide
CFSIWERLTPGDAC

SEQ ID NO: 24 - protein, 14 aa, Artificial Sequence
  Description of Artificial Sequence Synthetic peptide
ARAALWQRLTPGEI

SEQ ID NO: 25 - protein, 34 aa, Artificial Sequence
  Description of Artificial Sequence Synthetic peptide
GGGGGGGGGGGGGGGGGGGGARAALWQRLTPGEI

SEQ ID NO: 26 - protein, 33 aa, Artificial Sequence
  Description of Artificial Sequence Synthetic peptide
GGGGGGGGGGGGGGGGGGGARAALWQRLTPGEI

SEQ ID NO: 27 - protein, 32 aa, Artificial Sequence
  Description of Artificial Sequence Synthetic peptide
GGGGGGGGGGGGGGGGGGARAALWQRLTPGEI

SEQ ID NO: 28 - protein, 31 aa, Artificial Sequence
  Description of Artificial Sequence Synthetic peptide
GGGGGGGGGGGGGGGGGARAALWQRLTPGEI

SEQ ID NO: 29 - protein, 30 aa, Artificial Sequence
  Description of Artificial Sequence Synthetic peptide
GGGGGGGGGGGGGGGGARAALWQRLTPGEI

SEQ ID NO: 30 - protein, 29 aa, Artificial Sequence
  Description of Artificial Sequence Synthetic peptide
GGGGGGGGGGGGGGGARAALWQRLTPGEI

SEQ ID NO: 31 - protein, 28 aa, Artificial Sequence
  Description of Artificial Sequence Synthetic peptide
GGGGGGGGGGGGGGARAALWQRLTPGEI

SEQ ID NO: 32 - protein, 27 aa, Artificial Sequence
  Description of Artificial Sequence Synthetic peptide
GGGGGGGGGGGGGARAALWQRLTPGEI

SEQ ID NO: 33 - protein, 26 aa, Artificial Sequence
  Description of Artificial Sequence Synthetic peptide
GGGGGGGGGGGGARAALWQRLTPGEI

SEQ ID NO: 34 - protein, 25 aa, Artificial Sequence
  Description of Artificial Sequence Synthetic peptide
GGGGGGGGGGGARAALWQRLTPGEI

SEQ ID NO: 35 - protein, 24 aa, Artificial Sequence
  Description of Artificial Sequence Synthetic peptide
GGGGGGGGGGARAALWQRLTPGEI

SEQ ID NO: 36 - protein, 23 aa, Artificial Sequence
  Description of Artificial Sequence Synthetic peptide
GGGGGGGGGARAALWQRLTPGEI

SEQ ID NO: 37 - protein, 22 aa, Artificial Sequence
  Description of Artificial Sequence Synthetic peptide
GGGGGGGGARAALWQRLTPGEI

SEQ ID NO: 38 - protein, 21 aa, Artificial Sequence
  Description of Artificial Sequence Synthetic peptide
GGGGGGGARAALWQRLTPGEI

SEQ ID NO: 39 - protein, 20 aa, Artificial Sequence
  Description of Artificial Sequence Synthetic peptide
GGGGGGARAALWQRLTPGEI

SEQ ID NO: 40 - protein, 19 aa, Artificial Sequence
  Description of Artificial Sequence Synthetic peptide
GGGGGARAALWQRLTPGEI

SEQ ID NO: 41 - protein, 18 aa, Artificial Sequence
  Description of Artificial Sequence Synthetic peptide
GGGGARAALWQRLTPGEI

SEQ ID NO: 42 - protein, 17 aa, Artificial Sequence
  Description of Artificial Sequence Synthetic peptide
GGGARAALWQRLTPGEI

SEQ ID NO: 43 - protein, 14 aa, Artificial Sequence
  Description of Artificial Sequence Synthetic peptide
ARAALWQRLTPGEI

SEQ ID NO: 44 - protein, 14 aa, Artificial Sequence
  Description of Artificial Sequence Synthetic peptide
ARAALWQRLTPGEI

SEQ ID NO: 45 - protein, 14 aa, Artificial Sequence
  Description of Artificial Sequence Synthetic peptide
ARAALWQRLTPGEI

SEQ ID NO: 46 - protein, 14 aa, Artificial Sequence
  Description of Artificial Sequence Synthetic peptide
ARAALWQRLTPGEI

SEQ ID NO: 47 - protein, 14 aa, Artificial Sequence
  Description of Artificial Sequence Synthetic peptide
ARAALWQRLTPGEI

SEQ ID NO: 48 - protein, 14 aa, Artificial Sequence
  Description of Artificial Sequence Synthetic peptide
ARAALWQRLTPGEI

SEQ ID NO: 49 - protein, 14 aa, Artificial Sequence
  Description of Artificial Sequence Synthetic peptide
ARAALWQRLTPGEI

SEQ ID NO: 50 - protein, 14 aa, Artificial Sequence
  Description of Artificial Sequence Synthetic peptide
ARAALWQRLTPGEI

SEQ ID NO: 51 - protein, 14 aa, Artificial Sequence
  Description of Artificial Sequence Synthetic peptide
ARAALWQRLTPGEI

SEQ ID NO: 52 - protein, 14 aa, Artificial Sequence
  Description of Artificial Sequence Synthetic peptide
ARAALWQRLTPGEI

SEQ ID NO: 53 - protein, 34 aa, Artificial Sequence
  Description of Artificial Sequence Synthetic peptide
KKKKKKKKKKKKKKKKKKKKARAALWQRLTPGEI

SEQ ID NO: 54 - protein, 33 aa, Artificial Sequence
  Description of Artificial Sequence Synthetic peptide
KKKKKKKKKKKKKKKKKKKARAALWQRLTPGEI

SEQ ID NO: 55 - protein, 32 aa, Artificial Sequence
  Description of Artificial Sequence Synthetic peptide
KKKKKKKKKKKKKKKKKKARAALWQRLTPGEI

SEQ ID NO: 56 - protein, 31 aa, Artificial Sequence
  Description of Artificial Sequence Synthetic peptide
KKKKKKKKKKKKKKKKKARAALWQRLTPGEI

SEQ ID NO: 57 - protein, 30 aa, Artificial Sequence
  Description of Artificial Sequence Synthetic peptide
KKKKKKKKKKKKKKKKARAALWQRLTPGEI

SEQ ID NO: 58 - protein, 29 aa, Artificial Sequence
  Description of Artificial Sequence Synthetic peptide
KKKKKKKKKKKKKKKARAALWQRLTPGEI

SEQ ID NO: 59 - protein, 28 aa, Artificial Sequence
  Description of Artificial Sequence Synthetic peptide
KKKKKKKKKKKKKKARAALWQRLTPGEI

SEQ ID NO: 60 - protein, 27 aa, Artificial Sequence
  Description of Artificial Sequence Synthetic peptide
KKKKKKKKKKKKKARAALWQRLTPGEI

SEQ ID NO: 61 - protein, 26 aa, Artificial Sequence
  Description of Artificial Sequence Synthetic peptide
KKKKKKKKKKKKARAALWQRLTPGEI

SEQ ID NO: 62 - protein, 25 aa, Artificial Sequence
  Description of Artificial Sequence Synthetic peptide
KKKKKKKKKKKARAALWQRLTPGEI

SEQ ID NO: 63 - protein, 24 aa, Artificial Sequence
  Description of Artificial Sequence Synthetic peptide
KKKKKKKKKKARAALWQRLTPGEI

SEQ ID NO: 64 - protein, 23 aa, Artificial Sequence
  Description of Artificial Sequence Synthetic peptide
KKKKKKKKKARAALWQRLTPGEI

SEQ ID NO: 65 - protein, 22 aa, Artificial Sequence
  Description of Artificial Sequence Synthetic peptide
KKKKKKKKARAALWQRLTPGEI

SEQ ID NO: 66 - protein, 21 aa, Artificial Sequence
  Description of Artificial Sequence Synthetic peptide
KKKKKKKARAALWQRLTPGEI

SEQ ID NO: 67 - protein, 20 aa, Artificial Sequence
  Description of Artificial Sequence Synthetic peptide
KKKKKKARAALWQRLTPGEI

SEQ ID NO: 68 - protein, 19 aa, Artificial Sequence
  Description of Artificial Sequence Synthetic peptide
KKKKKARAALWQRLTPGEI

SEQ ID NO: 69 - protein, 18 aa, Artificial Sequence
  Description of Artificial Sequence Synthetic peptide
KKKKARAALWQRLTPGEI

SEQ ID NO: 70 - protein, 17 aa, Artificial Sequence
  Description of Artificial Sequence Synthetic peptide
KKKARAALWQRLTPGEI

SEQ ID NO: 71 - protein, 34 aa, Artificial Sequence
  Description of Artificial Sequence Synthetic peptide
ARAALWQRLTPGEIGGGGGGGGGGGGGGGGGGGG

SEQ ID NO: 72 - protein, 33 aa, Artificial Sequence
  Description of Artificial Sequence Synthetic peptide
ARAALWQRLTPGEIGGGGGGGGGGGGGGGGGGG

SEQ ID NO: 73 - protein, 32 aa, Artificial Sequence
  Description of Artificial Sequence Synthetic peptide
ARAALWQRLTPGEIGGGGGGGGGGGGGGGGGG

SEQ ID NO: 74 - protein, 31 aa, Artificial Sequence
  Description of Artificial Sequence Synthetic peptide
ARAALWQRLTPGEIGGGGGGGGGGGGGGGGG

SEQ ID NO: 75 - protein, 30 aa, Artificial Sequence
  Description of Artificial Sequence Synthetic peptide
ARAALWQRLTPGEIGGGGGGGGGGGGGGGG

SEQ ID NO: 76 - protein, 29 aa, Artificial Sequence
  Description of Artificial Sequence Synthetic peptide
ARAALWQRLTPGEIGGGGGGGGGGGGGGG

SEQ ID NO: 77 - protein, 28 aa, Artificial Sequence
  Description of Artificial Sequence Synthetic peptide
ARAALWQRLTPGEIGGGGGGGGGGGGGG

SEQ ID NO: 78 - protein, 27 aa, Artificial Sequence
  Description of Artificial Sequence Synthetic peptide
ARAALWQRLTPGEIGGGGGGGGGGGGG

SEQ ID NO: 79 - protein, 26 aa, Artificial Sequence
  Description of Artificial Sequence Synthetic peptide
ARAALWQRLTPGEIGGGGGGGGGGGG

SEQ ID NO: 80 - protein, 25 aa, Artificial Sequence
  Description of Artificial Sequence Synthetic peptide
ARAALWQRLTPGEIGGGGGGGGGGG

SEQ ID NO: 81 - protein, 24 aa, Artificial Sequence
  Description of Artificial Sequence Synthetic peptide
ARAALWQRLTPGEIGGGGGGGGGG

SEQ ID NO: 82 - protein, 23 aa, Artificial Sequence
  Description of Artificial Sequence Synthetic peptide
ARAALWQRLTPGEIGGGGGGGGG

SEQ ID NO: 83 - protein, 22 aa, Artificial Sequence
  Description of Artificial Sequence Synthetic peptide
ARAALWQRLTPGEIGGGGGGGG

SEQ ID NO: 84 - protein, 21 aa, Artificial Sequence
  Description of Artificial Sequence Synthetic peptide
ARAALWQRLTPGEIGGGGGGG

SEQ ID NO: 85 - protein, 20 aa, Artificial Sequence
  Description of Artificial Sequence Synthetic peptide
ARAALWQRLTPGEIGGGGGG

SEQ ID NO: 86 - protein, 19 aa, Artificial Sequence
  Description of Artificial Sequence Synthetic peptide
ARAALWQRLTPGEIGGGGG

SEQ ID NO: 87 - protein, 18 aa, Artificial Sequence
  Description of Artificial Sequence Synthetic peptide
ARAALWQRLTPGEIGGGG

SEQ ID NO: 88 - protein, 17 aa, Artificial Sequence
  Description of Artificial Sequence Synthetic peptide
ARAALWQRLTPGEIGGG

SEQ ID NO: 89 - protein, 14 aa, Artificial Sequence
  Description of Artificial Sequence Synthetic peptide
ARAALWQRLTPGEI

SEQ ID NO: 90 - protein, 14 aa, Artificial Sequence
  Description of Artificial Sequence Synthetic peptide
ARAALWQRLTPGEI

SEQ ID NO: 91 - protein, 14 aa, Artificial Sequence
  Description of Artificial Sequence Synthetic peptide
ARAALWQRLTPGEI

SEQ ID NO: 92 - protein, 14 aa, Artificial Sequence
  Description of Artificial Sequence Synthetic peptide
ARAALWQRLTPGEI

SEQ ID NO: 93 - protein, 14 aa, Artificial Sequence
  Description of Artificial Sequence Synthetic peptide
ARAALWQRLTPGEI

SEQ ID NO: 94 - protein, 14 aa, Artificial Sequence
  Description of Artificial Sequence Synthetic peptide
ARAALWQRLTPGEI

SEQ ID NO: 95 - protein, 14 aa, Artificial Sequence
  Description of Artificial Sequence Synthetic peptide
ARAALWQRLTPGEI

SEQ ID NO: 96 - protein, 14 aa, Artificial Sequence
  Description of Artificial Sequence Synthetic peptide
ARAALWQRLTPGEI

SEQ ID NO: 97 - protein, 14 aa, Artificial Sequence
  Description of Artificial Sequence Synthetic peptide
ARAALWQRLTPGEI

SEQ ID NO: 98 - protein, 14 aa, Artificial Sequence
  Description of Artificial Sequence Synthetic peptide
ARAALWQRLTPGEI

SEQ ID NO: 99 - protein, 34 aa, Artificial Sequence
  Description of Artificial Sequence Synthetic peptide
ARAALWQRLTPGEIKKKKKKKKKKKKKKKKKKKK

SEQ ID NO: 100 - protein, 33 aa, Artificial Sequence
  Description of Artificial Sequence Synthetic peptide
ARAALWQRLTPGEIKKKKKKKKKKKKKKKKKKK

SEQ ID NO: 101 - protein, 32 aa, Artificial Sequence
  Description of Artificial Sequence Synthetic peptide
ARAALWQRLTPGEIKKKKKKKKKKKKKKKKKK

SEQ ID NO: 102 - protein, 31 aa, Artificial Sequence
  Description of Artificial Sequence Synthetic peptide
ARAALWQRLTPGEIKKKKKKKKKKKKKKKKK

SEQ ID NO: 103 - protein, 30 aa, Artificial Sequence
  Description of Artificial Sequence Synthetic peptide
ARAALWQRLTPGEIKKKKKKKKKKKKKKKK

SEQ ID NO: 104 - protein, 29 aa, Artificial Sequence
  Description of Artificial Sequence Synthetic peptide
ARAALWQRLTPGEIKKKKKKKKKKKKKKK

SEQ ID NO: 105 - protein, 28 aa, Artificial Sequence
  Description of Artificial Sequence Synthetic peptide
ARAALWQRLTPGEIKKKKKKKKKKKKKK

SEQ ID NO: 106 - protein, 27 aa, Artificial Sequence
  Description of Artificial Sequence Synthetic peptide
ARAALWQRLTPGEIKKKKKKKKKKKKK

SEQ ID NO: 107 - protein, 26 aa, Artificial Sequence
  Description of Artificial Sequence Synthetic peptide
ARAALWQRLTPGEIKKKKKKKKKKKK

SEQ ID NO: 108 - protein, 25 aa, Artificial Sequence
  Description of Artificial Sequence Synthetic peptide
ARAALWQRLTPGEIKKKKKKKKKKK

SEQ ID NO: 109 - protein, 24 aa, Artificial Sequence
  Description of Artificial Sequence Synthetic peptide
ARAALWQRLTPGEIKKKKKKKKKK

SEQ ID NO: 110 - protein, 23 aa, Artificial Sequence
  Description of Artificial Sequence Synthetic peptide
ARAALWQRLTPGEIKKKKKKKKK

SEQ ID NO: 111 - protein, 22 aa, Artificial Sequence
  Description of Artificial Sequence Synthetic peptide
ARAALWQRLTPGEIKKKKKKKK

SEQ ID NO: 112 - protein, 21 aa, Artificial Sequence
  Description of Artificial Sequence Synthetic peptide
ARAALWQRLTPGEIKKKKKKK

SEQ ID NO: 113 - protein, 20 aa, Artificial Sequence
  Description of Artificial Sequence Synthetic peptide
ARAALWQRLTPGEIKKKKKK

SEQ ID NO: 114 - protein, 19 aa, Artificial Sequence
  Description of Artificial Sequence Synthetic peptide
ARAALWQRLTPGEIKKKKK

SEQ ID NO: 115 - protein, 18 aa, Artificial Sequence
  Description of Artificial Sequence Synthetic peptide
ARAALWQRLTPGEIKKKK

SEQ ID NO: 116 - protein, 17 aa, Artificial Sequence
  Description of Artificial Sequence Synthetic peptide
ARAALWQRLTPGEIKKK

SEQ ID NO: 117 - protein, 41 aa, Artificial Sequence
  Description of Artificial Sequence Synthetic peptide
GGGGGIEGPTLRQWLAARAGGGGGGGGARAALWQRLTPGEI

SEQ ID NO: 118 - protein, 14 aa, Artificial Sequence
  Description of Artificial Sequence Synthetic peptide
IEGPTLRQWLAARA

SEQ ID NO: 119 - protein, 14 aa, Artificial Sequence
  Description of Artificial Sequence Synthetic peptide
IEGPTLRQWLAARA

SEQ ID NO: 120 - protein, 14 aa, Artificial Sequence
  Description of Artificial Sequence Synthetic peptide
IEGPTLRQWLAARA

SEQ ID NO: 121 - protein, 41 aa, Artificial Sequence
  Description of Artificial Sequence Synthetic peptide
GGGGGARAALWQRLTPGEIGGGGGGGGIEGPTLRQWLAARA

SEQ ID NO: 122 - protein, 14 aa, Artificial Sequence
  Description of Artificial Sequence Synthetic peptide
ARAALWQRLTPGEI

SEQ ID NO: 123 - protein, 38 aa, Artificial Sequence
  Description of Artificial Sequence Synthetic peptide
ARAALWQRLTPGEIGGGGGGGGIEGPTLRQWLAARAIK

SEQ ID NO: 124 - protein, 14 aa, Artificial Sequence
  Description of Artificial Sequence Synthetic peptide
ARAALWQRLTPGEI

SEQ ID NO: 125 - protein, 41 aa, Artificial Sequence
  Description of Artificial Sequence Synthetic peptide
GGGGGARAALWQRLTPGEIGGGGGGGGARAALWQRLTPGEI

SEQ ID NO: 126 - protein, 14 aa, Artificial Sequence
  Description of Artificial Sequence Synthetic peptide
ARAALWQRLTPGEI

SEQ ID NO: 127 - protein, 37 aa, Artificial Sequence
  Description of Artificial Sequence Synthetic peptide
ARAALWQRLTPGEIGGGGGGGGARAALWQRLTPGEIK

SEQ ID NO: 128 - protein, 14 aa, Artificial Sequence
  Description of Artificial Sequence Synthetic peptide
ARAALWQRLTPGEI

SEQ ID NO: 129 - protein, 20 aa, Artificial Sequence
  Description of Artificial Sequence Synthetic peptide
GGGGGGGGGGGGGGGGGGGG

SEQ ID NO: 130 - protein, 19 aa, Artificial Sequence
  Description of Artificial Sequence Synthetic peptide
GGGGGGGGGGGGGGGGGGG

SEQ ID NO: 131 - protein, 18 aa, Artificial Sequence
  Description of Artificial Sequence Synthetic peptide
GGGGGGGGGGGGGGGGGG

SEQ ID NO: 132 - protein, 17 aa, Artificial Sequence
  Description of Artificial Sequence Synthetic peptide
GGGGGGGGGGGGGGGGG

SEQ ID NO: 133 - protein, 16 aa, Artificial Sequence
  Description of Artificial Sequence Synthetic peptide
GGGGGGGGGGGGGGGG

SEQ ID NO: 134 - protein, 15 aa, Artificial Sequence
  Description of Artificial Sequence Synthetic peptide
GGGGGGGGGGGGGGG

SEQ ID NO: 135 - protein, 14 aa, Artificial Sequence
  Description of Artificial Sequence Synthetic peptide
GGGGGGGGGGGGGG

SEQ ID NO: 136 - protein, 13 aa, Artificial Sequence
  Description of Artificial Sequence Synthetic peptide
GGGGGGGGGGGGG

SEQ ID NO: 137 - protein, 12 aa, Artificial Sequence
  Description of Artificial Sequence Synthetic peptide
GGGGGGGGGGGG

SEQ ID NO: 138 - protein, 11 aa, Artificial Sequence
  Description of Artificial Sequence Synthetic peptide
GGGGGGGGGGG

SEQ ID NO: 139 - protein, 10 aa, Artificial Sequence
  Description of Artificial Sequence Synthetic peptide
GGGGGGGGGG

SEQ ID NO: 140 - protein, 9 aa, Artificial Sequence
  Description of Artificial Sequence Synthetic peptide
GGGGGGGGG

SEQ ID NO: 141 - protein, 8 aa, Artificial Sequence
  Description of Artificial Sequence Synthetic peptide
GGGGGGGG

SEQ ID NO: 142 - protein, 7 aa, Artificial Sequence
  Description of Artificial Sequence Synthetic peptide
GGGGGGG

SEQ ID NO: 143 - protein, 6 aa, Artificial Sequence
  Description of Artificial Sequence Synthetic peptide
GGGGGG

SEQ ID NO: 144 - protein, 5 aa, Artificial Sequence
  Description of Artificial Sequence Synthetic peptide
GGGGG

SEQ ID NO: 145 - protein, 4 aa, Artificial Sequence
  Description of Artificial Sequence Synthetic peptide
GGGG

SEQ ID NO: 146 - protein, 20 aa, Artificial Sequence
  Description of Artificial Sequence Synthetic peptide
KKKKKKKKKKKKKKKKKKKK

SEQ ID NO: 147 - protein, 19 aa, Artificial Sequence
  Description of Artificial Sequence Synthetic peptide
KKKKKKKKKKKKKKKKKKK

SEQ ID NO: 148 - protein, 18 aa, Artificial Sequence
  Description of Artificial Sequence Synthetic peptide
KKKKKKKKKKKKKKKKKK

SEQ ID NO: 149 - protein, 17 aa, Artificial Sequence
  Description of Artificial Sequence Synthetic peptide
KKKKKKKKKKKKKKKKK

SEQ ID NO: 150 - protein, 16 aa, Artificial Sequence
  Description of Artificial Sequence Synthetic peptide
KKKKKKKKKKKKKKKK

SEQ ID NO: 151 - protein, 15 aa, Artificial Sequence
  Description of Artificial Sequence Synthetic peptide
KKKKKKKKKKKKKKK

SEQ ID NO: 152 - protein, 14 aa, Artificial Sequence
  Description of Artificial Sequence Synthetic peptide
KKKKKKKKKKKKKK

SEQ ID NO: 153 - protein, 13 aa, Artificial Sequence
  Description of Artificial Sequence Synthetic peptide
KKKKKKKKKKKKK

SEQ ID NO: 154 - protein, 12 aa, Artificial Sequence
  Description of Artificial Sequence Synthetic peptide
KKKKKKKKKKKK

SEQ ID NO: 155 - protein, 11 aa, Artificial Sequence
  Description of Artificial Sequence Synthetic peptide
KKKKKKKKKKK

SEQ ID NO: 156 - protein, 10 aa, Artificial Sequence
  Description of Artificial Sequence Synthetic peptide
KKKKKKKKKK

SEQ ID NO: 157 - protein, 9 aa, Artificial Sequence
  Description of Artificial Sequence Synthetic peptide
KKKKKKKKK

SEQ ID NO: 158 - protein, 8 aa, Artificial Sequence
  Description of Artificial Sequence Synthetic peptide
KKKKKKKK

SEQ ID NO: 159 - protein, 7 aa, Artificial Sequence
  Description of Artificial Sequence Synthetic peptide
KKKKKKK

SEQ ID NO: 160 - protein, 6 aa, Artificial Sequence
  Description of Artificial Sequence Synthetic peptide
KKKKKK

SEQ ID NO: 161 - protein, 5 aa, Artificial Sequence
  Description of Artificial Sequence Synthetic peptide
KKKKK

SEQ ID NO: 162 - protein, 4 aa, Artificial Sequence
  Description of Artificial Sequence Synthetic peptide
KKKK

SEQ ID NO: 163 - protein, 8 aa, Artificial Sequence
  Description of Artificial Sequence Synthetic peptide
GGGKGGGG

SEQ ID NO: 164 - protein, 27 aa, Artificial Sequence
  Description of Artificial Sequence Synthetic peptide
GGGGGXXXXXXXGGGGGGGGXXXXXXX

SEQ ID NO: 165 - protein, 22 aa, Artificial Sequence
  Description of Artificial Sequence Synthetic peptide
XXXXXXXGGGGGGGGXXXXXXX

SEQ ID NO: 166 - protein, 23 aa, Artificial Sequence
  Description of Artificial Sequence Synthetic peptide
XXXXXXXGGGGGGGGXXXXXXXK

SEQ ID NO: 167 - protein, 22 aa, Artificial Sequence
  Description of Artificial Sequence Synthetic peptide
XXXXXXXGGGKGGGGXXXXXXX

SEQ ID NO: 168 - protein, 41 aa, Artificial Sequence
  Description of Artificial Sequence Synthetic peptide
GGGGGIEGPTLRQWLAARAGGGGGGGGIEGPTLRQWLAARA

SEQ ID NO: 169 - protein, 36 aa, Artificial Sequence
  Description of Artificial Sequence Synthetic peptide
IEGPTLRQWLAARAGGGGGGGGIEGPTLRQWLAARA

SEQ ID NO: 170 - protein, 37 aa, Artificial Sequence
  Description of Artificial Sequence Synthetic peptide
IEGPTLRQWLAARAGGGGGGGGIEGPTLRQWLAARAK

SEQ ID NO: 171 - protein, 14 aa, Artificial Sequence
  Description of Artificial Sequence Synthetic peptide
IEGPTLRQWLAARA

SEQ ID NO: 172 - protein, 18 aa, Artificial Sequence
  Description of Artificial Sequence Synthetic peptide
IEGPTLRQWLAARAGGGK

SEQ ID NO: 173 - protein, 41 aa, Artificial Sequence; identical to SEQ ID NO: 168
  Description of Artificial Sequence Synthetic peptide

SEQ ID NO: 174 - protein, 41 aa, Artificial Sequence; identical to SEQ ID NO: 117
  Description of Artificial Sequence Synthetic peptide

SEQ ID NO: 175 - protein, 14 aa, Artificial Sequence
  Description of Artificial Sequence Synthetic peptide
IEGPTLRQWLAARA

SEQ ID NO: 176 - protein, 41 aa, Artificial Sequence; identical to SEQ ID NO: 121
  Description of Artificial Sequence Synthetic peptide

SEQ ID NO: 177 - protein, 14 aa, Artificial Sequence
  Description of Artificial Sequence Synthetic peptide
ARAALWQRLTPGEI

SEQ ID NO: 178 - protein, 38 aa, Artificial Sequence; identical to SEQ ID NO: 123
  Description of Artificial Sequence Synthetic peptide

SEQ ID NO: 179 - protein, 14 aa, Artificial Sequence
  Description of Artificial Sequence Synthetic peptide
ARAALWQRLTPGEI

SEQ ID NO: 180 - protein, 41 aa, Artificial Sequence; identical to SEQ ID NO: 125
  Description of Artificial Sequence Synthetic peptide

SEQ ID NO: 181 - protein, 14 aa, Artificial Sequence
  Description of Artificial Sequence Synthetic peptide
ARAALWQRLTPGEI

SEQ ID NO: 182 - protein, 37 aa, Artificial Sequence; identical to SEQ ID NO: 127
  Description of Artificial Sequence Synthetic peptide

SEQ ID NO: 183 - protein, 14 aa, Artificial Sequence
  Description of Artificial Sequence Synthetic peptide
ARAALWQRLTPGEI

SEQ ID NO: 184 - protein, 41 aa, Artificial Sequence
  Description of Artificial Sequence Synthetic peptide
GGGGGLQGPTLRAWRAGMAGGGGGGGGIEGPTLRQWLAARA

SEQ ID NO: 185 - protein, 41 aa, Artificial Sequence
  Description of Artificial Sequence Synthetic peptide
GGGGGIEGPTLRQWLAARAGGGGGGGGLQGPTLRAWRAGMA

SEQ ID NO: 186 - protein, 42 aa, Artificial Sequence
  Description of Artificial Sequence Synthetic peptide
LQGPTLRAWRAGMAGGGGGGGGIEGPTLRQWLAARAGGGGGK

SEQ ID NO: 187 - protein, 42 aa, Artificial Sequence
  Description of Artificial Sequence Synthetic peptide
IEGPTLRQWLAARAGGGGGGGIEGPTLRQWLAARAGGGGGKK

SEQ ID NO: 188 - protein, 41 aa, Artificial Sequence
  Description of Artificial Sequence Synthetic peptide
GGGGGLQGPTLRAWRAGMAGGGGGGGGLQGPTLRAWRAGMA

SEQ ID NO: 189 - protein, 19 aa, Artificial Sequence
  Description of Artificial Sequence Synthetic peptide
GGGGGLQGCTLRAWRAGMC

SEQ ID NO: 190 - protein, 36 aa, Artificial Sequence; identical to SEQ ID NO: 169
  Description of Artificial Sequence Synthetic peptide

SEQ ID NO: 191 - protein, 36 aa, Artificial Sequence
  Description of Artificial Sequence Synthetic peptide
LQGPTLRAWRAGMAGGGGGGGGIEGPTLRQWLAARA

SEQ ID NO: 192 - protein, 41 aa, Artificial Sequence; identical to SEQ ID NO: 184
  Description of Artificial Sequence Synthetic peptide

SEQ ID NO: 193 - protein, 41 aa, Artificial Sequence; identical to SEQ ID NO: 185
  Description of Artificial Sequence Synthetic peptide

SEQ ID NO: 194 - protein, 36 aa, Artificial Sequence; identical to SEQ ID NO: 169
  Description of Artificial Sequence Synthetic peptide

SEQ ID NO: 195 - protein, 36 aa, Artificial Sequence; identical to SEQ ID NO: 191
  Description of Artificial Sequence Synthetic peptide

SEQ ID NO: 196 - protein, 36 aa, Artificial Sequence; identical to SEQ ID NO: 169
  Description of Artificial Sequence Synthetic peptide

SEQ ID NO: 197 - protein, 37 aa, Artificial Sequence; identical to SEQ ID NO: 170
  Description of Artificial Sequence Synthetic peptide

SEQ ID NO: 198 - protein, 14 aa, Artificial Sequence
  Description of Artificial Sequence Synthetic peptide
IEGPTLRQWLAARA

SEQ ID NO: 199 - protein, 18 aa, Artificial Sequence
  Description of Artificial Sequence Synthetic peptide
IEGPTLRQWLAARAGGGK

SEQ ID NO: 200 - protein, 41 aa, Artificial Sequence; identical to SEQ ID NO: 168
  Description of Artificial Sequence Synthetic peptide

SEQ ID NO: 201 - protein, 36 aa, Artificial Sequence; identical to SEQ ID NO: 169
  Description of Artificial Sequence Synthetic peptide

SEQ ID NO: 202 - protein, 37 aa, Artificial Sequence; identical to SEQ ID NO: 170
  Description of Artificial Sequence Synthetic peptide

SEQ ID NO: 203 - protein, 14 aa, Artificial Sequence
  Description of Artificial Sequence Synthetic peptide
IEGPTLRQWLAARA

SEQ ID NO: 204 - protein, 18 aa, Artificial Sequence
  Description of Artificial Sequence Synthetic peptide
IEGPTLRQWLAARAGGGK

SEQ ID NO: 205 - protein, 4 aa, Artificial Sequence
  Description of Artificial Sequence Synthetic peptide
GGGK

SEQ ID NO: 206 - protein, 14 aa, Artificial Sequence
  Description of Artificial Sequence Synthetic peptide
LQGPTLRAWRAGMA

SEQ ID NO: 207 - protein, 14 aa, Artificial Sequence
  Description of Artificial Sequence Synthetic peptide
LQGCTLRAWRAGMC

SEQ ID NO: 208 - protein, 14 aa, Artificial Sequence
  Description of Artificial Sequence Synthetic peptide
AMGARWARLTPGQL

SEQ ID NO: 209 - protein, 14 aa, Artificial Sequence
  Description of Artificial Sequence Synthetic peptide
CMGARWARLTCGQL

SEQ ID NO: 210 - protein, 25 aa, Artificial Sequence
  Description of Artificial Sequence Synthetic peptide
GGGGGGGGGGGGKGGGGGGGGGGGG

SEQ ID NO: 211 - protein, 13 aa, Artificial Sequence
  Description of Artificial Sequence Synthetic peptide
KGGGGGGGGGGGG

SEQ ID NO: 212 - protein, 13 aa, Artificial Sequence
  Description of Artificial Sequence Synthetic peptide
GGGGGGGGGGGGK

SEQ ID NO: 213 - protein, 12 aa, Artificial Sequence
  Description of Artificial Sequence Synthetic peptide
GGGGGGGGGGGG

SEQ ID NO: 214 - protein, 12 aa, Artificial Sequence
  Description of Artificial Sequence Synthetic peptide
KKKKKKKKKKKK

SEQ ID NO: 215 - protein, 14 aa, Artificial Sequence
  Description of Artificial Sequence Synthetic peptide
IEGPTLRQWLAARA

SEQ ID NO: 216 - protein, 36 aa, Artificial Sequence
  Description of Artificial Sequence Synthetic peptide
IEGPTLRQWLAARAGGGKGGGGIEGPTLRQWLAARA

SEQ ID NO: 217 - protein, 21 aa, Artificial Sequence
  Description of Artificial Sequence Synthetic peptide
GGGGGGGGGGKGGGGGGGGGG

SEQ ID NO: 218 - protein, 11 aa, Artificial Sequence
  Description of Artificial Sequence Synthetic peptide
KGGGGGGGGGG

SEQ ID NO: 219 - protein, 11 aa, Artificial Sequence
  Description of Artificial Sequence Synthetic peptide
GGGGGGGGGGK

SEQ ID NO: 220 - protein, 35 aa, Artificial Sequence
  Description of Artificial Sequence Synthetic peptide
XXXXXXXGGGGGGGGGGKGGGGGGGGGGXXXXXXX

SEQ ID NO: 221 - protein, 25 aa, Artificial Sequence
  Description of Artificial Sequence Synthetic peptide
XXXXXXXKGGGGGGGGGGXXXXXXX

SEQ ID NO: 222 - protein, 25 aa, Artificial Sequence
  Description of Artificial Sequence Synthetic peptide
XXXXXXXGGGGGGGGGGKXXXXXXX

SEQ ID NO: 223 - protein, 54 aa, Artificial Sequence
  Description of Artificial Sequence Synthetic peptide
GGGGGXXXXXXXGGGGGGGGXXXXXXXGGGGGXXXXXXXGGGGGGGGXXXXXXX

SEQ ID NO: 224 - protein, 23 aa, Artificial Sequence
  Description of Artificial Sequence Synthetic peptide
KXXXXXXXGGGGGGGGXXXXXXX

SEQ ID NO: 225 - protein, 15 aa, Artificial Sequence
  Description of Artificial Sequence Synthetic peptide
ARAALWQRLTPGEIK

SEQ ID NO: 226 - protein, 14 aa, Artificial Sequence
  Description of Artificial Sequence Synthetic peptide
ARAALWQRLTPGEI

SEQ ID NO: 227 - protein, 15 aa, Artificial Sequence
  Description of Artificial Sequence Synthetic peptide
ARAALWQRLTPGEIK

SEQ ID NO: 228 - protein, 14 aa, Artificial Sequence
  Description of Artificial Sequence Synthetic peptide
IEGPTLRQWLAARA

SEQ ID NO: 229 - protein, 15 aa, Artificial Sequence
  Description of Artificial Sequence Synthetic peptide
IEGPTLRQWLAARAK

SEQ ID NO: 230 - protein, 14 aa, Artificial Sequence
  Description of Artificial Sequence Synthetic peptide
ARAALWQRLTPGEI

SEQ ID NO: 231 - protein, 15 aa, Artificial Sequence
  Description of Artificial Sequence Synthetic peptide
ARAALWQRLTPGEIK

SEQ ID NO: 232 - protein, 18 aa, Artificial Sequence
  Description of Artificial Sequence Synthetic peptide
GGGGIEGPTLRQWLAARA

SEQ ID NO: 233 - protein, 15 aa, Artificial Sequence
  Description of Artificial Sequence Synthetic peptide
ARAALWQRLTPGEIK

SEQ ID NO: 234 - protein, 14 aa, Artificial Sequence
  Description of Artificial Sequence Synthetic peptide
IEGPTLRQWLAARA

SEQ ID NO: 235 - protein, 15 aa, Artificial Sequence
  Description of Artificial Sequence Synthetic peptide
IEGPTLRQWLAARAK

SEQ ID NO: 236 - protein, 14 aa, Artificial Sequence
  Description of Artificial Sequence Synthetic peptide
ARAALWQRLTPGEI

SEQ ID NO: 237 - protein, 15 aa, Artificial Sequence
  Description of Artificial Sequence Synthetic peptide
ARAALWQRLTPGEIK

SEQ ID NO: 238 - protein, 14 aa, Artificial Sequence
  Description of Artificial Sequence Synthetic peptide
IEGPTLRQWLAARA

SEQ ID NO: 239 - protein, 18 aa, Artificial Sequence
  Description of Artificial Sequence Synthetic peptide
GGGGIEGPTLRQWLAARA

SEQ ID NO: 240 - protein, 14 aa, Artificial Sequence
  Description of Artificial Sequence Synthetic peptide
IEGPTLRQWLAARA

SEQ ID NO: 241 - protein, 18 aa, Artificial Sequence
  Description of Artificial Sequence Synthetic peptide
GGGGIEGPTLRQWLAARA

SEQ ID NO: 242 - protein, 4 aa, Artificial Sequence
  Description of Artificial Sequence Synthetic peptide
GGGG

SEQ ID NO: 243 - protein, 41 aa, Artificial Sequence; identical to SEQ ID NO: 168
  Description of Artificial Sequence Synthetic peptide

(19) United States
(12) Patent Application Publication
(10) Pub. No.: US 2009/0054332 A1
(43) Pub. Date: Feb. 26, 2009

(54) THOMBOPOIETIN PEPTIDE CONJUGATES

(75) Inventors: Jean-Philippe Estradier, Montreal (CA); Martin Robitaille, St. Colomban (CA); Karen Thibaudeau, Rosemere (CA); Dominique P. Bridon, San Francisco, CA (US); Xicai Huang, Belle Mead, NJ (US); Omar Quraishi, Westmount (CA); Nathalie Bousquet-Gagnon, Lachine (CA)

Correspondence Address: LOWRIE, LANDO & ANASTASI, LLP, ONE MAIN STREET, SUITE 1100, CAMBRIDGE, MA, 02142, US

(73) Assignee: CONJUCHEM BIOTECHNOLOGIES, INC., Montreal (CA)

(21) Appl. No.: 12/143,780

(22) Filed: Jun. 21, 2008

Related U.S. Application Data
(60) Provisional application No. 60/936,843, filed on Jun. 21, 2007.
(60) Provisional application No. 60/967,279, filed on Aug. 30, 2007.

(51) Int. Cl. A61K 38/16; C07K 7/06; C07K 7/08; C07K 1/14; C07K 14/76; C07K 19/00

(52) U.S. Cl. 514/12; 530/329; 530/326; 530/327; 530/362; 530/391.7; 530/344

(57) ABSTRACT

The present invention relates to compounds comprising modified thrombopoietin peptides and conjugates of such modified peptides to serum components, typically serum proteins or peptides. The compounds and conjugates of the invention comprise a reactive group or a residue of a reactive group, which is covalently attached to a modified peptide, optionally through a linking group. The present invention also provides methods for the covalent attachment of a modified peptide to a serum protein or peptide to form a conjugate of the invention. The conjugates of the invention typically exhibit a longer in vivo circulating half-life compared to the corresponding unconjugated peptides. The conjugates of the invention also retain at least some of the biological activity of the unconjugated peptides, and typically exhibit increased biological activity compared to the unconjugated peptides. The present invention also provides methods for the treatment and prevention of a disease or disorder comprising the administration of one or more of the compounds or conjugates of the invention to a subject in need of such treatment or prevention.

### CROSS REFERENCE TO RELATED APPLICATIONS

This application claims priority to U.S. Ser. No. 60/936,843, filed on Jun. 21, 2007, and U.S. Ser. No. 60/967,279, filed on Aug. 30, 2007. The contents of the aforementioned applications are hereby incorporated by reference in their entirety. This application also incorporates by reference the International Application filed with the U.S. Receiving Office on Jun. 21, 2008, entitled “Thrombopoietin Peptide Conjugates” and bearing attorney docket number C2077-7017WO.

### BACKGROUND OF THE INVENTION

Thrombopoietin (TPO) is a glycoprotein hormone involved in the regulation of platelet production. Thrombopoietin is believed to be capable of promoting the proliferation of megakaryocyte progenitors in the bone marrow and their maturation into platelet-producing megakaryocytes.

Thrombopoietin could have therapeutic value in treatment of patients with reduced platelet count. For instance, cancer patients can suffer thrombocytopenias on account of myelosuppressive chemotherapy. Such patients conventionally have been treated by platelet transfusion. Thrombopoietin can stimulate immature blood cells and megakaryocytes to develop into platelet-producing megakaryocytes.

At least two forms of recombinant human thrombopoietin have been tested in clinical trails. One is a truncated version comprising the N-terminal 163 amino acids of TPO conjugated with polyethylene glycol and is known as pegylated recombinant human megakaryocyte growth and development factor (PEG-rHuMDGF). The other is a full length, glycosylated molecule known as recombinant human thrombopoietin (rhTPO).

Both forms of recombinant thrombopoietin have been evaluated in Phase I and Phase I/II clinical trials in cancer patients receiving chemotherapy in order to boost platelet counts. Basser et al., 1996, Lancet 348:1279-1281; Vadhan-Raj et al., 1996, Blood 88:448a; Basser et al., 1997, Blood 89:118-3128; Fannuchi et al., 1997, New England Journal of Medicine 336:404-409; Vadhan-Raj et al. 1997, Ann. Intern. Med. 126:673-681; Vadhan-Raj S. et al., 1998, Semin. Hematol. 35:261-268; Vadhan-Raj et al., 2000, Ann Intern Med 132:364-8; Vadhan-Raj et al., 2003, J Clin Oncol 21:3158-67; Angiolillo et al., 2005, Clin. Cancer Res. 11(7):2644-50.

Both forms of TPO have been found to be immunogenic in a small proportion of subjects, and neutralizing antibodies have also been demonstrated to both molecules. Hardy et al., 1997, Toxicologist 36:277; Li et al., 2001, Blood 98:3241-3248; Koren, 2002, Dev Biol 109:87-95; Basser et al., 2002, Blood 99:2599-2602; Koren, 2002, Current Pharmaceutical Biotechnology 3:349-360. Side effects have included thrombocytosis and thrombosis. However, recent phase I trials have demonstrated safe administration of rhTPO in adult and pediatric populations. Angiolillo et al., 2005, Clin. Cancer Res. 11(7):2644-50; Wolff et al., 2001, Bone Marrow Transplant 27:261-8.

In a phase III study, PEG-rHuMDGF was found not consistently efficacious in reducing the duration of severe thrombocytopenia. Schuster et al., 2002, Exp. Hematol. 30: 1044-50.

Other forms of thrombopoietin have been described, for example, in Cwirla et al., 1997, Science 276:1696-1698 and in U.S. Pat. No. 6,465,430.

In some recipients of platelet transfusion, platelet refractoriness is observed. This means that their platelet count increases by less than what would be expected for the quantity of platelets they have received. This results in the need for additional and more frequent transfusions to reach and maintain a minimally acceptable level of platelets in their blood.

Platelet refractoriness is often observed in patients receiving multiple transfusions and is also more common in women who have been pregnant.

Frequently, refractoriness results from platelet alloimmunization, the generation of antibodies against platelet membrane glycoproteins. Refractoriness can also result from non-alloimmune factors including but not limited to ABO blood-type incompatibility, ITP (immune thrombocytopenic purpura), sepsis, malaria, or hemolytic anemia, alone or in combination with alloimmunization. In general, the more transfusions one has received, the higher the risk of developing refractoriness through alloimmunization. In one study (Kiefel et al., 2001, Transfusion 41:766-770), platelet-reactive antibodies were detected in 45% of 252 multi-transfused patients.

In some 70-80% of refractoriness cases, antibodies are against HLA class I antigens (human leukocyte antigens). In a further 10-20%, they are against HPA (platelet-specific antigens). Lastly, in some 5-10% of cases, both types are observed.

Recently, platelet donations have been treated by leukoreduction, the reduction of the number of leukocytes contaminating the collected platelets through filtering or irradiation, in order to minimize the risk of infectious disease transmission by leukocyte-associated infectious agents or alloimmunization. This has reduced but not eliminated the incidence of platelet alloimmunization.

Matching for both AB 0 blood-type and HLA has a 1-in-4 chance amongst family relatives and a 1-in-10,000 chance among random donors, which could result in difficulty in finding a platelet match, especially in view of the short storage shelf-life for platelets of 3-5 days at 22° C.

To date a need still exists for safe, effective forms of thrombopoietin useful, for example, for treatment of thrombocytopenia.

### SUMMARY OF THE INVENTION

The present invention provides a compound comprising a modified thrombopoietin peptide wherein the peptide is modified by the covalent attachment of a reactive group to the peptide, wherein the covalent attachment is optionally through a linking group, and wherein the reactive group is capable of forming a covalent bond with an amino, hydroxyl, or thiol group of a serum protein. The present invention also provides a modified thrombopoietin peptide wherein the peptide is covalently attached to a reactive group, wherein the covalent attachment is optionally through a linking group, and wherein the reactive group is capable of forming a covalent bond with an amino, hydroxyl, or thiol group of a serum protein. The present invention further provides a modified thrombopoietin peptide consisting of a peptide covalently attached to a reactive group, wherein the covalent attachment is optionally through a linking group, and wherein the reactive group is capable of forming a covalent bond with an amino, hydroxyl, or thiol group of a serum protein.

A thrombopoietin peptide of the invention is any peptide that is capable of binding a thrombopoietin receptor as detected by any assay known to those of skill in the art, and that displays one or more biological activities of thrombopoietin, including but not limited to, regulating proliferation and differentiation of megakaryocytes, and production of platelets. In some embodiments of the peptides, compounds or conjugates of the invention, the amino acid sequence of the peptide comprises or consists of a sequence selected from the group consisting of SEQ ID NOs: 1-128, 168-204, and 206-209. In other embodiments of the peptides, compounds or conjugates of the invention, the peptide is a derivative of the peptide comprising a sequence selected from the group consisting of SEQ ID NOs: 1-128, 168-204, and 206-209. Unless stated otherwise, the amino acid sequences and formulae depicted herein are shown in a configuration from N-terminus to C-terminus.

The present invention also provides a conjugate comprising a modified thrombopoietin peptide, wherein the peptide is covalently linked to a residue of a reactive group, optionally through a linking group, and wherein the residue of the reactive group is covalently linked to a serum protein. Such a conjugate can be formed, for example, by reacting a modified thrombopoietin peptide of the invention with a serum protein under conditions in which the reactive group of the modified thrombopoietin peptide is capable of reacting with the serum protein to form said conjugate.

The peptides of the invention are modified by the covalent attachment of one or more reactive groups to the peptide. Typically, each peptide is modified by the covalent attachment of one reactive group to a defined amino acid of the peptide. A reactive group is chosen for its ability to form a covalent bond with another peptide or protein, thus enabling the covalent attachment of the modified peptide to the other peptide or protein through the reactive group. A residue of a reactive group refers to the chemical structure resulting from covalent bond formation between the reactive group and another moiety, e.g., a peptide or protein such as a peptide or protein present in blood. Typically, the reactive group is capable of forming a covalent bond with a peptide or protein present in blood, referred to herein as a serum protein or peptide. As used herein, reference to a blood or serum protein (or peptide) means that the protein or peptide can be found in the blood or serum, but for purposes of the invention need not be isolated from the blood or serum. Thus, a blood or serum protein (or peptide) as used herein can be chemically synthesized or recombinantly produced. In a specific embodiment, the serum protein is albumin (e.g., human serum albumin), transferrin, or ferritin, or an immunoglobulin such as IgM or IgG. In one embodiment, the serum protein is recombinant. In one embodiment, the serum protein is recombinantly produced albumin, typically recombinant human albumin.

The reactive group can be linked to the peptide directly or via an optional linking group. The optional linking group can be any linking group apparent to those of skill in the art. The linking group can be a biocompatible polymer with a length of 1 to 100 atoms. As described herein, the length of a linking group is expressed by the number of atoms in the shortest chain of atoms between the groups linked by the linking group. In certain embodiments, the biocompatible polymer is a linker having a length of 1 to 100 atoms that, e.g., includes or consists of polyglycine, polylysine, polyglutamate, polyisoleucine, or polyarginine residues, or a combination thereof. For example, the polyglycine or polylysine linkers can include at least two, three, four, five, six, eight, nine, ten, eleven or twelve glycine or lysine residues, or a combination thereof. In other embodiments, the polyglycine or polylysine linkers can include no more than two, three, four, five, six, eight, nine, ten, eleven or twelve glycine or lysine residues. Exemplary linking groups include, but are not limited to, polyglycine, polylysine, polyglutamate, polyisoleucine, polyarginine or other suitable linking groups including two or more amino acids where the amino acids are the same or different. In some embodiments, the amino acid linking groups can include at least two, three, four, five, six, eight, nine, ten, eleven or twelve amino acid residues, for example, glycine, lysine, glutamate, isoleucine and/or arginine residues. In other embodiments, the amino acid linking groups can include no more than two, three, four, five, six, eight, nine, ten, eleven or twelve amino acid residues, for example, glycine, lysine, glutamate, isoleucine and/or arginine residues. Examples of such a polyglycine that includes a lysine, e.g., a single lysine, have, for example, an amino acid sequence of (G)ₐK(G)_{b}, K(G)c, and (G)_{d}K, where a, b, c and d are each an integer greater than or equal to one, for example, one, two, three, four, five, six, seven, eight, nine, ten, eleven, twelve or more. In some examples, a and b may be the same, whereas in other examples a and b are not the same. For example, a may be three and b may be four to provide a linking group having an amino acid sequence of GGGKGGGG (SEQ ID NO:163). In some examples, a lysine, e.g., the single lysine, within the polyglycine linker may itself be linked to one or more additional moieties, e.g., a linking group, a reactive group, a residue of a reactive group linked to a protein (e.g., a serum protein). For example, the lysine may be linked to one or more additional linkers through, for example, the epsilon amino group of the side chain.

In other embodiments, the linking group includes a biocompatible polymer, e.g., a polymer having monomers chosen from one or more of AEA ((2-amino) ethoxy acetic acid), AEEA ([2-(2-amino)ethoxy)]ethoxy acetic acid) or OA (8-amino octanoic acid, also called 8-amino caprylic acid, of formula NH₂—(CH₂)₇—COOH), or a combination thereof. In certain embodiments, the biocompatible polymer is an AEA polymer having a length of 1 to 100 atoms. In certain embodiments, the biocompatible polymer is an AEEA polymer having a length of 1 to 100 atoms. In certain embodiments, the biocompatible polymer is an OA polymer having a length of 1 to 100 atoms. Illustrative examples of linking groups include monomers, dimers, trimers, tetramers, pentamers, sixmers, septamers, octamers, nonamer, decamer, undecamers, dodecamers of glycine, lysine, glutamate, isoleucine, or arginine residues, AEA, AEEA or OA (e.g., an OA dimer (-OA-OA-) or an OA trimer (-OA-OA-OA-), or any combination thereof (e.g., any combination of Glyₙ, Lysₙ, OA, AEA, or AEEA, wherein n=1, 2, 3, 4, 5, 6, 7, 8, 9, 10, 11, or 12). In some embodiments, the linking group has an amino acid, e.g., a single lysine or K. The amino acid, e.g., the single lysine, can be modified to be linked to one or more additional moieties, e.g., a linking group, a reactive group, a residue of a reactive group linked to a protein (e.g., a serum protein). For example, the single lysine may be linked to the reactive group or one or more additional linkers through, for example, the epsilon amino group of the side chain. Examples of such linkers including a single lysine have, for example, the sequence of (monomer)ₐK(monomer)_{b}, K(monomer)c, and (monomer)_{d}K, where a, b, c and d are each an integer greater than or equal to one, for example, one, two, three, four, five, six, seven, eight, nine, ten, eleven, twelve or more. In some examples, a and b may be the same, whereas in other examples a and b are not the same. For example, a may be three and b may be four to provide a linking group having the following sequence: (OA)ₙK(OA)ₙ, (OA)ₙK(AEA)ₙ, (G)ₙK(OA)ₙ, (OA)ₙK(G)ₙ, wherein n=1, 2, 3, 4, 5, 6, 7, 8, 9, 10, 11, or 12. In the case where the reactive group is located on the linker between the two TMPs, the total length between the two TMPs will not typically exceed 100 atoms. The attachments to the linker group are not considered to be part of the linker between the two TMPs.

The linking group can include any combinations of the aforesaid biocompatible polymers. For example, a polyglycine linker can be combined with one or more monomers of AEA, AEEA or OA in any configuration. In one embodiment, the polyglycine linker is attached to one or more monomers of AEA, AEEA or OA at either the N- or the C-terminal end of the first or last glycine residue in the polyglycine linker. Alternatively one or more monomers of AEA, AEEA or OA are inserted in between glycine residues in the polyglycine linker.

In particular embodiments, the reactive group (for example, MPA, GMBA, NHS, sulfo-NHS, MBS or GMBS), is attached to the peptide through one or more linking groups, including, for example, a polyglycine linker, a polyglycine-lysine linker, AEEA, AEA, or OA, or any combination thereof. In certain embodiments in which the reactive group is attached to the peptide through more than one linking group, each linking group can be independently selected from the group consisting typically of, polyglycine, polylysine, AEA, AEEA, and OA. In embodiments, the number of linking groups (e.g., monomeric polymer units) is from 1 to 2, 1 to 3, 1 to 4, 1 to 5, 1 to 6, 1 to 7, 1 to 8, 1 to 9, 1 to 10, 1 to 11, 1 to 12. Where there is more than one linking group, the linking groups can be the same or different linking groups. For example, any combination of one of polyglycine, polylysine, AEA, AEEA, and/or OA can be used in any order. In one embodiment, the reactive group, typically MPA, is attached to the peptide via 1, 2, 3, 4, 5, 6, 7, 8, 9, 10, 11, or 12 polyglycine, polylysine, AEA, AEEA or OA linking groups which are arranged in tandem. In another embodiment, the reactive group, typically MPA, is attached to the peptide via 1, 2, 3, 4, 5, 6, 7, 8, 9, 10, 11, or 12 polyglycine, polylysine, AEA, AEEA or OA linking groups which are arranged in a branched configuration.

The reactive group can be linked directly or via the linking group to any site of the peptide deemed suitable according to one of skill in the art. In certain embodiments, the reactive group is linked directly or indirectly to the backbone of the peptide. In certain embodiments, the reactive group is linked directly or indirectly to the N-terminus, e.g., the N-terminal amine, of the peptide. In certain embodiments, the reactive group is linked directly or indirectly to the C-terminus, e.g., the C-terminal carboxyl, of the peptide. In certain embodiments, the reactive group is linked directly or indirectly to a side chain of the peptide, e.g., a side chain hydroxyl, thiol, amino or carboxyl, of the peptide. In other embodiments, the reactive group is linked directly or indirectly to the epsilon amino group of a lysine side chain of the peptide. In yet other embodiments, the reactive group is linked to any site in the linking group that is connecting two thrombopoietin peptides (e.g., the reactive group is linked to a linking group that connects an end or an internal side chain of a first thrombopoietin peptide to an end or an internal side chain of a second thrombopoietin peptide). In one embodiment, the reactive group is linked to a linking group that connects the C-terminal carboxyl end of a first thrombopoietin peptide to the N-terminal end of a second thrombopoietin peptide.

In certain embodiments, a compound of the invention can comprise more than one thrombopoietin peptide. The thrombopoietin peptides can be linked in a linear manner via amide bonds along the peptide chain. They can be linked via a C-terminal to C-terminal linkage, an N-terminal to N-terminal linkage, an N-terminal to C-terminal linkage, or via a mixture thereof (i.e. when the compound comprises three or more thrombopoietin peptides). The thrombopoietin peptides can also be linked via an internal side chain or via any linkage apparent to those of skill in the art.

In embodiments that comprise more than one thrombopoietin peptide, the thrombopoietin peptides can be linked directly or via one or more optional linking groups, e.g., a linking group as described herein. An optional linking group can be any linking group apparent to those of skill in the art. For instance, the linking group can be a biocompatible polymer with a length of 1 to 100 atoms. As described herein, the length of a linking group is expressed by the number of atoms in the shortest chain of atoms between the groups linked by the linking group. In certain embodiments, the biocompatible polymer is a polyglycine, polylysine, polyglutamate, polyisoleucine, polyarginine, each one having a length of 1 to 100 atoms or other suitable linking groups including two or more amino acids where the amino acids are the same or different. For example, the polymer linkers can include at least two, three, four, five, six, eight, nine, ten, eleven or twelve glycine, lysine, glutamate, isoleucine or polyarginine residues. In other embodiments, the biocompatible polymer is a polymer having monomers selected from AEA, AEEA and OA. In certain embodiments, the biocompatible polymer is an AEA, AEEA or OA polymer having a length of 1 to 100 atoms. Illustrative examples of linking groups include monomers, dimers, trimers, tetramers, pentamers, sixmers, septamers, octamers, nonamer, decamer, undecamers, dodecamers of glycine, lysine, glutamate, isoleucine, or arginine residues, AEA, AEEA or OA (e.g., an OA dimer (-OA-OA-) or an OA trimer (-OA-OA-OA-), or any combination thereof (e.g., any combination of Glyₙ, Lysₙ, OA, AEA, or AEEA, wherein n=1, 2, 3, 4, 5, 6, 7, 8, 9, 10, 11, or 12). In one embodiment, the polyglycine linker is attached to one or more monomers of AEA, AEEA or OA at either the N- or the C-terminal end of the first or last glycine residue in the polyglycine linker. Alternatively one or more monomers of AEA, AEEA or OA are inserted in between glycine residues in the polyglycine linker. In other embodiments, the linking group is a peptide having a labile chemical bond which is cleavable by an enzyme or which is cleavable under acidic conditions. In other embodiments, the linking group has a single lysine. The single lysine can be modified to be linked to a reactive group directly or via one or more linking groups. For example, the K group may be linked to the reactive group or one or more additional linkers through, for example, the epsilon amino group of the side chain. Examples of such linkers including a single lysine have, for example, the sequence of (monomer)ₐK(monomer)_{b}, K(monomer)c, and (monomer)_{d}K, where a, b, c and d are each an integer greater than or equal to one, for example, one, two, three, four, five, six, seven, eight, nine, ten, eleven, twelve or more. In some examples, a and b may be the same, whereas in other examples a and b are not the same. For example, a may be three and b may be four to provide a linking group having the following sequence: (OA)ₙK(OA)ₙ, (OA)ₙK(AEA)ₙ, (G)ₙK(OA)ₙ, (OA)ₙK(G)ₙ, wherein n=1, 2, 3, 4, 5, 6, 7, 8, 9, 10, 11, or 12. In the case where the reactive group is located on the linker between the two TMPs, the total length between the two TMPs will not typically exceed 100 atoms. The attachments to the linker group are not considered to be part of the linker between the two TMPs.

The present invention also provides conjugates that are composed of modified peptides that are covalently attached to a serum protein through a reactive group. Thus, a conjugate of the present invention comprises a modified thrombopoietin peptide, in which the reactive group has formed a covalent bond to a serum protein.

The present invention also provides methods for the covalent attachment of a modified peptide to a serum protein to form the conjugates of the invention. In one embodiment, a modified peptide of the invention is covalently bound to a serum protein in vitro (ex vivo), by an in vitro reaction of the serum protein with the modified peptide comprising the reactive group, such that the modified peptide is covalently attached to the serum protein through a residue of the reactive group. In another embodiment, a modified peptide of the invention is covalently bound to a serum protein in vivo, by an in vivo reaction of the serum protein with the modified peptide comprising the reactive group, such that the modified peptide is covalently attached to the serum protein through a residue of the reactive group.

In one embodiment of the compound or conjugate of the invention, the serum protein is selected from the group consisting of albumin, transferrin, ferritin, IgM, IgG, a steroid binding protein, a thyroxin binding protein, and alpha-2-macroglobulin.

In one embodiment of the compound or conjugate of the invention, the covalent attachment of the reactive group is at substantially only one site on the peptide.

In one embodiment of the conjugate of the invention, the covalent attachment of the reactive group is at substantially only one site on the peptide and the reactive group is covalently attached to the serum protein at substantially only one site on the protein. In one embodiment, the serum protein is albumin. In a specific embodiment, the covalent attachment of the reactive group is at the epsilon amino group of a lysine in the peptide, optionally through a linking group, and a residue of the reactive group is covalently attached to albumin through a maleimide group to the thiol group of cysteine 34 of albumin. Typically, the maleimide group is part of a maleimido propionic acid (MPA).

In one embodiment of the compound or conjugate of the invention, the serum protein is albumin, typically human albumin. In one embodiment, the serum protein is recombinant, typically recombinant albumin, and most typically recombinant human albumin.

In one embodiment of the compound or conjugate of the invention, the reactive group is a maleimide containing group, typically gamma-maleimide-butylamide (GMBA), maleimido propionic acid (MPA), N-hydroxysuccinimide (NHS), N-hydroxy-sulfosuccinimide (sulfo-NHS), maleimide-benzoyl-succinimide (MBS) or gamma-maleimido-butyryloxy succinimide ester (GMBS).

In certain embodiments, a modified peptide (Formula Ia) or conjugate (Formula Ib) of the invention is according to the following formula:

TMP-LL (optionally)-reactive group (Formula Ia)

TMP-LL (optionally)-residue-X-protein (Formula Ib)

wherein TMP is a thrombopoietin peptide, e.g., a thrombopoietin peptide as described herein; LL is an optional linker, e.g., a biocompatible linker as disclosed herein; reactive group is a coupling group as described herein, e.g., MPA; residue is a residue of a reactive group following conjugation; protein is a serum protein and X is a —S—, —N(H)— or —O— from said protein, e.g. from an amino acid side chain. As described herein, the residue or the linker can be linked to any site of the thrombopoietin peptide, including, for example, the N-terminus, the C-terminus or an amino acid side chain. In other embodiments, the residue or the linker is linked to the epsilon amino group of a lysine side chain in the peptide, which lysine can be present in the native peptide or added to the N- or C-terminus of the peptide or inserted or substituted into the native peptide. In certain embodiments, the residue is covalently coupled to the linker, e.g., a biocompatible linker as disclosed herein.

In other embodiments, a modified peptide (Formula Ia, IIa, or IVa) or a conjugate (Formula IIb, IIIb or IVb) of the invention is according a formula chosen from one of the following:

TMP₁-LL₁-TMP₂-LL₂ (optionally)—reactive group (Formula IIa);

TMP₁-LL₁-TMP₂-LL₂ (optionally)—residue-X-protein (Formula IIb);

Reactive group-LL₂ (optionally)-TMP₁-LL₁-TMP₂ (Formula IIa);

Protein-X-residue-LL₂ (optionally)-TMP₁-LL₁-TMP₂ (Formula IIIb);

TMP₁-LL₁-LL₂ (optionally) [reactive group]-TMP₂ (Formula IVa);

TMP₁-LL₁-LL₂ (optionally) [residue-X-protein]-TMP₂ (Formula IVb);

also represented herein as Formula IVc:

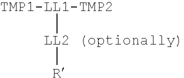

wherein TMP₁ and TMP₂ are each independently a thrombopoietin peptide as described herein (any combination, orientation or order of a TMP peptide as described herein can be used); each LL₁ and LL₂ are each independently a linker, e.g., a biocompatible linker as disclosed herein; reactive group is a coupling group as described herein, e.g., MPA; residue is a residue of a reactive group following conjugation, R′ can be either a reactive group or a residue of a reactive group following conjugation; protein is a serum protein, and X is a —S—, —N(H)— or —O— from said protein, e.g. from an amino acid side chain. LL₂ linker may be optional. In embodiments where LL₂ is not present, the reactive group is linked directly to the N-terminus, the C-terminus or an amino acid side chain of TMP₁ and TMP₂. As described herein, the linker can be linked to any site of the thrombopoietin peptide, including, for example, the N-terminus, the C-terminus or an amino acid side chain. In specific embodiments, the linker is linked to the epsilon amino group of a lysine side chain in the peptide, which lysine can be present in the native peptide or added to the N- or C-terminus of the peptide or inserted or substituted into the native peptide. In other embodiments, an amino acid residue is covalently coupled to the linker, e.g., a biocompatible linker as disclosed herein. For example, the LL₁ linker can contain one amino acid, e.g., a lysine, residue. The amino acid, e.g., lysine, residue of LL₁ can be linked to a reactive group directly or optionally via a linker, LL₂. In embodiments of Formula IV where the lysine residue is in linker LL₁, the epsilon amino group of a lysine side chain can be covalently linked to one or more moieties, e.g., a linking group (e.g., LL₂), a reactive group, or a residue of a reactive group coupled to a protein, e.g., albumin. Examples of such linkers include—(monomerₙ-K[(optional monomer)ₙ (reactive group)](optional monomer)ₙ, wherein n=1 to 12, and the monomer can be any of glycine, lysine, glutamate, isoleucine, or arginine residues, AEA, AEEA or OA.

Exemplary modified peptides (Formula Va, VIa, VIIa, VIIIa or IXa) or conjugates (Formula Vb, VIIb, VIIb, VIIb or IXb) include or consist of the following formula:

Reactive Group-(Gly₅)-TMP₁-(Gly₈)-TMP₂ (SEQ ID NO: 164) (Formula Va);

Protein-X-Residue-(Gly₅)-TMP₁-(Gly₈)-TMP₂ (SEQ ID NO: 164) (Formula Vb);

TMP₁-(Gly₈)-TMP₂—(OA₂)-K-reactive group (SEQ ID NO: 165) (Formula VIa);

TMP₁-(Gly₈)-TMP₂—(OA₂)-K-residue-X-protein (SEQ ID NO: 165), (Formula VIb);

TMP₁-(Gly₈)-TMP₂-K-(OA₂)-reactive group (SEQ ID NO: 166) (Formula VIIIa);

TMP₁-(Gly₈)-TMP₂-K-(OA₂)-residue-X-protein (SEQ ID NO: 166) (Formula VIb);

Reactive group-(OA₂)-(TMP₁)-(OA₃)-(TMP₂) (Formula VIIIa);

Protein-X-residue-(OA₂)-(TMP₁)—(OA₃)-(TMP₂) (Formula VIIb);

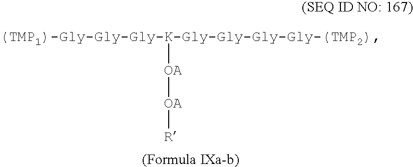

wherein

TMP₁ and TMP₂ are each a peptide having a sequence of X¹ X² X³ X⁴ X⁵ X⁶ X⁷, where X¹ is C, L, M, P, Q or V; X² is F, K, L, N, Q, R, S, T or V; X³ is C, F, I, L, M, R, S, V or W; X⁴ is any of the 20 genetically coded L-amino acids; X⁵ is A, D, E, G, K, M, Q, R, S, T, V or Y; X⁶ is C, F, G, L, M, S, V, W or Y; and X⁷ is C, G, I, K, L, M, N, R or V. Any combination, orientation, or order of a TMP peptide as described herein can be used. In one embodiment, TMP₁ and TMP₂ are each a peptide having a sequence IEGPTLRQWLAARA (SEQ ID NO:11);

OA is an 8-aminooctanoyl linking group;

R′ is either a reactive group, e.g., MPA (Formula IXa), or a residue of a reactive group following conjugation (Formula IXb);

protein is a serum protein and X is a —S—, —N(H)— or —O— from said protein, e.g. from an amino acid side chain;

Gly or G is glycine;

K is lysine and the reactive group is linked to the lysine through the lysine's epsilon amino group to form the reactive group residue. In some examples, the reactive group may be removed and substituted by a linking group that can react with the C-terminus of the lysine group. Illustrative such linking groups are capable of forming an amide bond between the C-terminus of the lysine and an amino group of the linker. One such linking is a derivatized reactive group that includes a primary amino group.

Specific examples of modified peptides or conjugates include or consist of the following sequences:

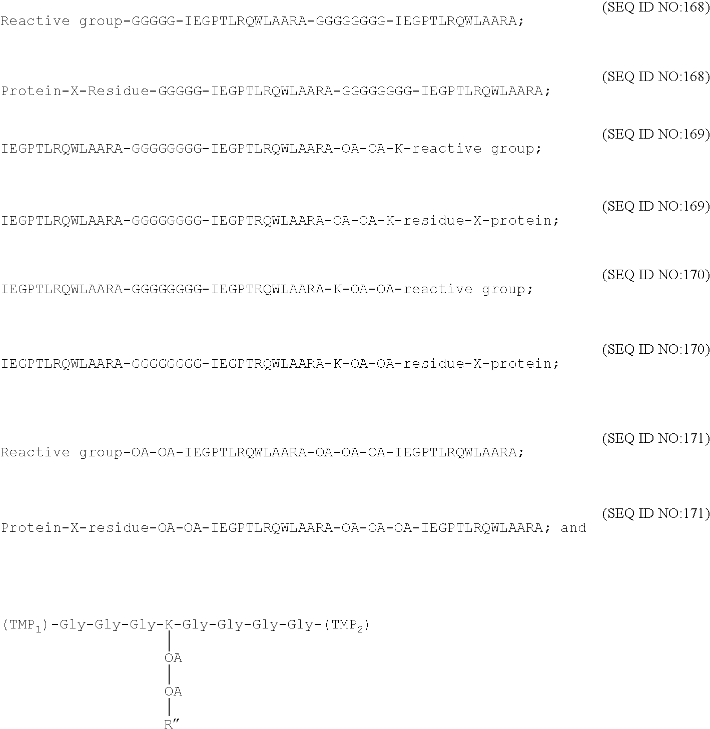

(SEQ ID NO:167), wherein R″ is either a reactive group, or a residue of a reactive group following conjugation; TMP₁ and TMP₂ are each a peptide having a sequence IEGPTLRQWLAARA (SEQ ID NO:11). Although the spacing between amino acids is sometimes indicated as ‘-’, unless indicated otherwise, the peptide and linker sequence described herein are intended to be contiguous.

In one embodiment of the compound or conjugate of the invention, the peptide is a derivative having up to 4 amino acid substitutions, insertions, or deletions relative to the sequence of any of SEQ ID NOS 1-128, 168-204, and 206-209.

In one embodiment of the compound or conjugate of the invention, the peptide is a mammalian peptide or derivative. In a specific embodiment, the peptide is selected from the group consisting of a mouse, rat, guinea pig, rabbit, dog, horse, cow, pig, or primate peptide or derivative. In one embodiment, the peptide is a human peptide or derivative.

In one embodiment of the conjugate of the invention, the conjugate has a decreased rate of excretion and/or an increased circulating half-life in vivo compared to a corresponding unconjugated peptide. In one embodiment, the conjugate retains at least some of the biological activity of the unconjugated peptide. In another embodiment, the conjugate exhibits more biological activity than the unconjugated peptide. In certain embodiments, the biological activity is the ability to modulate megakaryocyte or platelet production. In certain embodiments, the biological activity is the ability to bind to a thrombopoietin receptor such as, for example, cMP-1.

The invention also provides a method for extending the in vivo half-life of a modified peptide of the invention, the method comprising contacting the modified peptide containing a reactive group with a serum protein under conditions suitable for the formation of a covalent bond between the reactive group and the serum protein, thereby forming a modified peptide residue conjugated to the serum protein, which modified peptide residue conjugated to the serum protein has an increased in vivo half-life relative to the unconjugated modified peptide. In one embodiment, the contacting is performed in vivo. In one embodiment, the contacting is performed in vitro. In embodiments, the serum protein is albumin, typically human, and most typically recombinant human albumin.

The invention also provides a method for modifying the biological activity of a thrombopoietin peptide by forming a conjugate of the invention, either in vivo by administration of a compound (comprising a modified peptide or modified derivative with a reactive group as described above) of the invention or forming a conjugate of the invention ex vivo, as described herein, for example, by separating action on peripheral and central receptors following the formation of a covalent bond between the reactive group residue and the serum protein. In one embodiment, the method allows access of the conjugate of the invention to peripheral receptors only so as to directly act in an agonist or antagonist manner.

The invention also provides a method for treating or preventing a disease or disorder in a subject, the method comprising administering to the subject an amount of the compound or conjugate of the invention effective for said treating or preventing. In one embodiment, the disease or disorder is thrombocytopenia. The thrombocytopenia can be caused by any cause of thrombocytopenia known to those of skill in the art. In certain embodiments, the thrombocytopenia is caused by cancer therapy. In certain embodiments, the thrombocytopenia is immune thrombocytopenic purpura. In certain embodiments, the thrombocytopenia is caused by myelodysplastic syndrome or leukemia. In certain embodiments, the thrombocytopenia is caused by a disease or condition damaging the liver such as hepatitis or cirrhosis. In certain embodiments, the thrombocytopenia is caused by infectious diseases such as HIV infection. In certain embodiments, the thrombocytopenia is caused by trauma or surgery such as liver transplantation or cardiopulmonary bypass. In certain embodiments, the thrombocytopenia is caused by substances such as heparin.

The invention also provides a method for harvesting platelets in a subject, the method comprising administering to the subject an amount of the compound or conjugate of the invention and harvesting platelets from the subject. In certain embodiments, the harvested platelets are administered to a recipient to prevent or treat thrombocytopenia. In one embodiment, the donor is the recipient of the harvested platelets. In another embodiment, the donor is a relative of the recipient of the harvested platelets. In another embodiment, the donor is unrelated to the recipient of the harvested platelets. In a further embodiment, multiple recipients can be administered the harvested platelets, and these can include the donor, its relatives, and unrelated persons in any combination.

The invention also provides a method for harvesting and administering platelets in a subject with the aim of preventing or providing reduced or absent refractoriness to platelet transfusion, the method comprising administering to a subject an amount of a compound or conjugate of the invention and harvesting platelets from the donor prior to administering the platelets to a recipient. In certain embodiments, the recipient is refractory to platelet transfusion. In certain embodiments, the recipient is not refractory to platelet transfusion but is about to undergo, or has already started to undergo multiple platelet transfusions. In certain embodiments, the recipient is not refractory to platelet transfusion, but has an infrequent combination of ABO blood-type, HLA (human leukocyte antigens), and HPA (platelet-specific antigens). In certain embodiments, donor and recipient are matched for ABO blood-type and HLA compatibility. In certain further embodiments, donor and recipient are matched for ABO blood-type, HLA, and HPA (platelet-specific antigens) compatibility. In other embodiments, donor and recipient are matched for HLA compatibility and/or HPA compatibility, and optionally for ABO blood-type compatibility.

The invention also provides a pharmaceutical composition comprising a compound or conjugate of the invention and a pharmaceutically acceptable carrier, excipient, or diluent.

In a specific embodiment, the compounds or conjugates of the invention are purified. In a specific embodiment, the compounds or conjugates of the invention that are used in the methods of the invention and/or that are present in the pharmaceutical compositions of the invention are purified.

The invention also provides a kit comprising in one or more containers a compound or conjugate of the invention. In one embodiment, the compound or conjugate is in lyophilized form. In accordance with this embodiment, the kit further comprises a container of sterile solution suitable for reconstituting the lyophilized compound or conjugate.

The invention also provides a syringe containing in a solution a compound or conjugate of the invention.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

All publications, patent applications, patents, and other references mentioned herein are incorporated by reference in their entirety.

Other features, objects, and advantages of the invention will be apparent from the description and drawings, and from the claims.

### DETAILED DESCRIPTION OF THE INVENTION

The present invention provides compounds comprising modified thrombopoietin peptide, and conjugates comprising such residues of modified peptides covalently attached to a blood component, for example, a serum protein or peptide.

### Thrombopoietin Derivatives and Conjugates

A thrombopoietin peptide of the invention is any peptide that is capable of binding a thrombopoietin receptor as detected by any assay known to those of skill in the art, and that displays one or more biological activities of thrombopoietin, including but not limited to, regulating proliferation and differentiation of megakaryocytes, and production of platelets. In certain embodiments, the amino acid sequence of the thrombopoietin peptide comprises or consists of SEQ ID NO:1:

X¹ X² X³ X⁴ X⁵ X⁶ X⁷

where X¹ is C, L, M, P, Q or V; X² is F, K, L, N, Q, R, S, T or V; X³ is C, F, I, L, M, R, S, V or W; X⁴ is any of the 20 genetically coded L-amino acids; X⁵ is A, D, E, G, K, M, Q, R, S, T, V or Y; X⁶ is C, F, G, L, M, S, V, W or Y; and X⁷ is C, G, I, K, L, M, N, R or V. The thromopoietin peptide can be oriented from N-terminus to C-terminus, or from C-terminus to N-terminus.

In certain embodiments, the amino acid sequence of the thrombopoietin peptide comprises or consists of SEQ ID NO:2:

X⁸ G X¹ X² X³ X⁴ X⁵ W X⁷

where X¹ is C, L, M, P, Q, or V; X² is F, R, S, or T; X³ is F, L, V, or W; X is A, K, L, M, R, S, V, or T; X⁵ is A, E, G, K, M, Q, R, S, or T; X⁷ is C, I, K, L, M, R or V; and each X⁸ residue is independently selected from any of the 20 genetically coded L-amino acids, their stereoisomeric D-amino acids; and non-natural amino acids. In certain embodiments, each X⁸ residue is independently selected from any of the 20 genetically coded L-amino acids and their stereoisomeric D-amino acids. In certain embodiments (SEQ ID NO:3), X¹ is P; X² is T; X³ is L; X⁴ is R; X⁵ is E or Q; and X⁷ is I or L. The thromopoietin peptide can be oriented from N-terminus to C-terminus, or from C-terminus to N-terminus.

Exemplary thrombopoietin peptides include peptides whose amino acid sequences comprise or consist of any of SEQ ID NOs: 4-11 or a derivative thereof:

(SEQ ID NO:4)

G G C A D G P T L R E W I S F C G G;

(SEQ ID NO:5)

G N A D G P T L R Q W L E G R R P K N;

(SEQ ID NO:6)

G G C A D G P T L R E W I S F C G G K;

(SEQ ID NO:7)

T I K G P T L R Q W L K S R E H T S;

(SEQ ID NO:8)

S I E G P T L R E W L T S R T P H S;

(SEQ ID NO:9)

L A I E G P T L R Q W L H G N G R D T;

(SEQ ID NO:10)

C A D G P T L R E W I S F C;

(SEQ ID NO:11)

I E G P T L R Q W L A A R A;

(SEQ ID NO:206)

L Q G P T L R A W R A G M A;
and

(SEQ ID NO:207)

L Q G C T L R A W R A G M C.

The thromopoietin peptide can be oriented from N-terminus to C-terminus, or from C-terminus to N-terminus.

In certain embodiments, the amino acid sequence of the thrombopoietin peptide comprises or consists of SEQ ID NO:12:

X⁹ X⁸ G X¹ X² X³ X⁴ X⁵ W X⁷

where X¹ is C, L, M, P, Q, or V; X² is F, R, S, or T; X³ is F, L, V, or W; X⁴ is A, K, L, M, R, S, V, or T; X⁵ is A, E, G, K, M, Q, R, S, or T; X⁷ is C, I, K, L, M R, or V; X⁹ is A, C, E, G, I, L, M, P, R, Q, S, T, or V; and X⁸ is A, C, D, E, K, L, Q, R, S, T, or V. In certain embodiments, X⁹ is A or I; and X⁸ is D, E, or K. The thromopoietin peptide can be oriented from N-terminus to C-terminus, or from C-terminus to N-terminus. In certain embodiments, the amino acid sequence of the thrombopoietin peptide comprises or consists of SEQ ID NO: 13:

X⁷ X⁶ X⁵ X⁴ X³ X² X¹

where X¹ is C, L, M, P, Q or V; X² is F, K, L, N, Q, R, S, T or V; X³ is C, F, I, L, M, R, S, V or W; X⁴ is any of the 20 genetically coded L-amino acids; X⁵ is A, D, E, G, K, M, Q, R, S, T, V or Y; X⁶ is C, F, G, L, M, S, V, W or Y; and X⁷ is C, G, I, K, L, M, N, R or V. The thromopoietin peptide can be oriented from N-terminus to C-terminus, or from C-terminus to N-terminus.

In certain embodiments, the amino acid sequence of the thrombopoietin peptide comprises or consists of SEQ ID NO:14:

X⁷ W X⁵ X⁴ X³ X² X¹ G X⁸

where X¹ is C, L, M, P, Q, or V; X² is F, R, S, or T; X is F, L, V, or W; X is A, K, L, M, R, S, V, or T; X⁵ is A, E, G, K, M, Q, R, S, or T; X⁷ is C, I, K, L, M, R, or V; and each X⁸ residue is independently selected from any of the 20 genetically coded L-amino acids, their stereoisomeric D-amino acids; and non-natural amino acids. In certain embodiments, each X⁸ residue is independently selected from any of the 20 genetically coded L-amino acids and their stereoisomeric D-amino acids. In certain embodiments (SEQ ID NO:15), X¹ is P; X² is T; X³ is L; X⁴ is R; X⁵ is E or Q; and X⁷ is I or L. The thromopoietin peptide can be oriented from N-terminus to C-terminus, or from C-terminus to N-terminus.

In certain embodiments, the amino acid sequence of the thrombopoietin peptide comprises or consists of SEQ ID NO:16:

X⁷ W X⁵ X⁴ X³ X² X¹ G X⁸ X⁹

where X¹ is C, L, M, P, Q, or V; X² is F, R, S, or T; X³ is F, L, V, or W; X is A, K, L, M, R, S, V, or T; X⁵ is A, E, G, K, M, Q, R, S, or T; X⁷ is C, I, K, L, M, R, or V; X⁸ is A, C, D, E, K, L, Q, R, S, T, or V; X⁹ is A, C, E, G, I, L, M, P, R, Q, S, T, or V. In certain embodiments, X⁹ is A or I; and X⁸ is D, E, or K. The thromopoietin peptide can be oriented from N-terminus to C-terminus, or from C-terminus to N-terminus.

Exemplary thrombopoietin peptides include the peptides whose amino acid sequences comprise or consist of any of SEQ ID NOs: 17-24 or a derivative thereof:

(SEQ ID NO:17)

G G C F S I W E R L T P G D A C G G;

(SEQ ID NO:18)

N K P R R G E L W Q R L T P G D A N G;

(SEQ ID NO:19)

K G G C F S I W E R L T P G D A C G G;

(SEQ ID NO:20)

S T H E R S K L W Q R L T P G K I T;

(SEQ ID NO:21)

S H P T R S T L W E R L T P G E I S;

(SEQ ID NO:22)

T D R G N G H L W Q R L T P G E I A L;

(SEQ ID NO:23)

C F S I W E R L T P G D A C;

(SEQ ID NO:24)

A R A A L W Q R L T P G E I;

(SEQ ID NO:208)

AMGARWARLTPGQL;
and

(SEQ ID NO:209)

CMGARWARLTCGQL.

The thromopoietin peptide can be oriented from N-terminus to C-terminus, or from C-terminus to N-terminus.

In certain embodiments, the thrombopoietin peptide is any thrombopoietin peptide described in, e.g., U.S. Pat. No. 6,465,430 and by Kimura et al. (1997) J Biochem. 122:1046-51, the contents of which are hereby incorporated by reference in their entirety.

In certain embodiments, a thrombopoietin peptide, is a peptide that binds to a thrombopoietin receptor (“cMP-1”) or a member of its family of subtypes. Exemplary thrombopoietin assays include those described in U.S. Pat. No. 6,465,430, the contents of which are hereby incorporated by reference in their entirety. In certain embodiments, the thrombopoietin peptide is capable of binding a thrombopoietin receptor with an IC₅₀ of less than 2 mM, less than 1 mM, less than 500 μM, less than 250 μM, less than 100 μM, less than 50 μM, less than 25 μM, less than 10 μM, less than 5 μM, less than 2.5 μM or less than 1 μM.

In certain embodiments, a thrombopoietin peptide is a mammalian peptide, specifically, a mouse, rat, guinea pig, rabbit, dog, cat, horse, cow, pig, or primate peptide, or derivative thereof. Typically, the peptide is a human peptide, or derivative thereof.

The thrombopoietin peptides of the invention (as present in the modified peptides, conjugates or compounds of the invention) are typically amidated at the C-terminus and/or acylated at the N-terminus.

As used herein, a “derivative” of a peptide refers to a compound wherein the amino acid sequence of the compound is the same as that of the peptide except for up to 10, typically up to 8, up to 6, up to 5, up to 4, up to 3, up to 2, or up to 1 amino acid insertions, deletions, and/or substitutions of the amino acid sequence of the peptide. Typically, a derivative binds to the same biological receptor as the peptide and thus displays at least some of the biological activity of the peptide.

The amino acid notations used herein for the twenty genetically encoded L-amino acids are conventional and are as follows:

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically indicated otherwise, the three-letter amino acid abbreviations designate amino acids in the L-configuration. Specific enantiomers are preceded with a “D-” or “L-”, depending upon the enantiomer. Unless specifically indicated otherwise, the capital one-letter abbreviations refer to amino acids in the L-configuration.

Unless noted otherwise, when polypeptide sequences are presented as a series of one-letter and/or three-letter abbreviations, the sequences are presented in the amino to carboxy terminal direction, in accordance with common practice.

As used herein, “genetically encoded amino acid” refers to L-isomers of the twenty amino acids that are defined by genetic codons. The genetically encoded amino acids are the L-isomers of glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine and lysine.

A modified peptide, conjugate or compound of the invention comprises a reactive group covalently attached to the peptide. The reactive group is chosen for its ability to form a stable covalent bond with a serum protein or peptide, for example, by reacting with one or more amino groups, hydroxyl groups, or thiol groups on the serum protein or peptide. Typically, a reactive group reacts with only one amino group, hydroxyl group, or thiol group on the serum protein or peptide. Typically, a reactive group reacts with a specific amino group, hydroxyl group, or thiol group on the serum protein or peptide. A conjugate of the invention comprises a modified peptide, which is covalently attached to a serum protein or peptide via a reaction of the reactive group with an amino group, hydroxyl group, or thiol group on the serum protein or peptide. Thus, a conjugate of the invention comprises a modified peptide, in which a residue of the reactive group has formed a covalent bond to a serum protein or peptide. As used herein, “a residue of a reactive group” or “a reactive group residue” refers to the chemical structure resulting from covalent bond formation between the reactive group and another moiety, e.g., a peptide or protein present in blood. In embodiments of the modified peptides, conjugates or compounds of the invention, the reactive group is a maleimide containing group selected from gamma-maleimide-butrylamide (GMBA), maleimido propionic acid (MPA), N-hydroxysuccinimide (NHS), N-hydroxy-sulfosuccinimide (sulfo-NHS), maleimide-benzoyl-succinimide (MBS) and gamma-maleimido-butyryloxy succinimide ester (GMBS).

As used herein, a reference to blood or serum proteins (or peptides) means that the protein or peptide can be found in the blood or serum but for purposes of the invention need not be isolated from the blood or serum. Thus, a blood or serum protein (or peptide) as used herein can be chemically synthesized or recombinantly produced. In a specific embodiment, the serum protein is selected from the group consisting of albumin, transferrin, ferritin, IgM, or IgG. In another embodiment, the serum protein is a steroid binding protein, a thyroxin binding protein, or alpha-2-macroglobulin. In certain embodiments, the modified peptides of the invention are conjugated to cells present in the blood, such as erythrocytes or platelets. In a preferred embodiment, the serum protein is albumin, typically recombinant albumin, and most typically human.

As used herein, “albumin” refers to the most abundant protein in blood plasma having a molecular weight of approximately between 65 and 67 kilodaltons in its monomeric form, depending on the species of origin. The term “albumin” is used interchangeably with “serum albumin” and is not meant to define the source of the albumin which forms a conjugate with the modified peptides of the invention. Thus, the term “albumin” as used herein may refer either to albumin purified from a natural source such as blood or serous fluids, or it may refer to chemically synthesized or recombinantly produced albumin.

In various embodiments, albumin variants or derivatives of native albumins can be used for formation of conjugates with the modified peptides of the invention. In some embodiments, the albumin is a mammalian albumin, or a variant or derivative thereof. Non-limiting examples of mammalian albumins that can be used include human, bovine, ovine, caprine, rabbit, feline, canine, porcine, primate, or rodent albumin. In a preferred embodiment, the mammalian albumin is human albumin. In one embodiment, the human albumin is purified from blood or serous fluids. In another embodiment, the albumin is recombinant albumin. In a particular embodiment, the albumin is recombinant human albumin (referred to herein as “rHA”). In various embodiments, rHA can be produced in a mammalian or non-mammalian organism. In one embodiment, the rHA is produced in a non-mammalian organism. Examples of non-mammalian organisms that can be used for the production of rHA include, without limitation, yeast, bacteria, plants, fungi, and insects. In one embodiment, the rHA is produced in a whole plant or a whole fungus. In another embodiment, the rHA is produced in cultured plant cells, cultured fungus cells, or cultured insect cells. In another embodiment, the rHA is produced in a non-human mammal or in non-human mammalian cells. Examples of non-human mammals that can be used for the production of rHA include, without limitation, those belonging to one of the following: the family Bovidae, the family Canidae, the family Suidae, the order Rodentia, the order Lagomorpha, and the order Primates (excluding humans). In a particular embodiment, the non-human mammal that is used for the production of rHA is selected from the group consisting of a cow, a dog, a pig, a sheep, a goat, a rat, a mouse, a rabbit, a chimpanzee, and a gorilla. In another embodiment, the non-human mammalian cells used for the production of rHA are, without limitation, bovine, canine, porcine, ovine, caprine, rodent, rabbit, or non-human primate cells. The main advantage of rHA produced in a non-human organism compared with albumin purified from human blood or serous fluids is the absence of human-derived products in the manufacturing process of rHA. The use of such controlled production methods leads to a purer product with less structural heterogeneity. Previous studies have indicated that there is no significant difference between soluble rHA and human albumin purified from blood or serous fluids in terms of their biochemical characteristics, radiolabelling efficiency and biological behavior in vitro and in vivo. See Dodsworth et al., 1996, Biotechnol. Appl. Biochem. 24: 171-176. In a particular embodiment, the albumin is the rHA designated by the trade name RECOMBUMIN® (Novozymes Inc., Nottingham, UK). RECOMBUMIN® is a recombinant human albumin that is produced in vitro using recombinant yeast technology, in which genetically modified yeast (Saccharomyces cerevisiae) secrete soluble rHA which is subsequently harvested, purified and formulated for use as an excipient for the manufacture of biologics or a coating for medical devices.

Reactive groups suitable for covalent attachment to thrombopoietin peptide are discussed in more detail below. In certain embodiments, more than one reactive group is attached to the thrombopoietin peptide or linker. Typically, a single reactive group is attached to a particular defined amino acid of the thrombopoietin peptide, or monomer or peptide of the linker. In one embodiment, a single reactive group is attached to the peptide at a particular defined amino acid of the thrombopoietin peptide or linker. In a specific embodiment, a single maleimide containing group, typically maleimido propionic acid (MPA), is attached to the peptide at a particular defined amino acid of the peptide. The reactive group can be attached to the thrombopoietin peptide or a linking group by any method or technique known to those of skill in the art. Exemplary methods or techniques are described in U.S. Pat. No. 6,849,714, the content of which is hereby incorporated by reference in its entirety.

For example, the reactive group can be linked to any site of the thrombopoietin peptide or a linking group deemed suitable according to one of skill in the art. In certain embodiments, the reactive group is linked to the backbone of the peptide or derivative. In certain embodiments, the reactive group is linked to the N-terminus, e.g., the N-terminal amine, of the peptide or derivative. In certain embodiments, the reactive group is linked to the C-terminus, e.g., the C-terminal carboxyl, of the peptide or derivative. In certain embodiments, the reactive group is linked to a side chain of the peptide or derivative, e.g., a side chain hydroxyl, thiol, amino or carboxyl, of the peptide or derivative. In specific embodiments, the reactive group is linked to the epsilon amino group of a lysine side chain of the peptide or derivative.

In other embodiments, the reactive group is bound to the peptide via one or more intervening molecules, or “linking groups” (also referred to herein as “linkers”). In certain embodiments, the linking group is a biocompatible polymer, e.g., a peptide or an alkyl or an alkoxy containing polymer. In a specific embodiment, the linking group is a peptide having a labile chemical bond which is cleavable by an enzyme or which is cleaved under specific chemical conditions, e.g., acidic conditions. In one embodiment, the modified peptide comprises a reactive group covalently attached to the peptide through one or more linking groups. In certain embodiments, the linking group includes one or more reactive groups, typically one linking group. In certain embodiments, the linking group has a length of 1 to 100 atoms. As described herein, the length of a linking group is expressed by the number of atoms in the shortest chain of atoms between the groups linked by the linking group. In certain embodiments, the linking group has from 1 to 100 atoms, from 1 to 80 atoms, from 1 to 60 atoms, from 1 to 50 atoms, from 1 to 40 atoms, from 1 to 30 atoms, from 1 to 20 atoms, from 10 to 20 atoms or from 5 to 15 atoms. Where more than one linking group is present, the linking groups may be the same or different linking groups. The linking group can be attached to the thrombopoietin peptide by any method or technique known to those of skill in the art. Exemplary methods or techniques are described in U.S. Pat. No. 6,849,714, the contents of which are hereby incorporated by reference in its entirety.

Exemplary peptide linking groups include, but are not limited to, polyglycine, polyglutamate, polyisoleucine, polyarginine or other suitable linking groups including two or more amino acids. In some examples, the amino acid linking groups can include at least two, three, four, five, six, eight, nine, ten, eleven or twelve amino acid residues, for example, glycine or lysine residues. A polyglycine linker can include one or more different residues (e.g., lysine residues) inserted in any configuration, e.g., near the N- or C-terminal end, or in the middle of a stretch of glycine residues. In other embodiments, a polyglycine linker is combined with one or more monomers of AEA, AEEA or OA in any configuration. In one embodiment, the polyglycine linker is attached to one or more monomers of AEA, AEEA or OA at either the N- or the C-terminal end of the first or last glycine residue in the polyglycine linker. Alternatively one or more monomers of AEA, AEEA or OA are inserted in between glycine residues in the polyglycine linker. In examples, where a polyglycine is used as a linker, the polyglycine may include a single lysine to provide a free epsilon amino group capable of reacting with another linker or with a protein. Examples of such a polyglycine that includes an amino acid, e.g., a single lysine, have, for example, an amino acid sequence of (G)ₐK(G)_{b}, K(G)c, and (G)_{d}K. where a, b, c and d are each an integer greater than or equal to one, for example, one, two, three, four, five, six, seven, eight, nine, ten, eleven, twelve or more. In some examples, a and b may be the same, whereas in other examples a and b are not the same. For example, a may be three and b may be four to provide a linking group having an amino acid sequence of GGGKGGGG (SEQ ID NO:163). In some examples, the single lysine within the polyglycine linker may itself be linked to additional moieties, e.g., a linking group, a reactive group or a residue of a reactive group. For example, the lysine residue may be linked to one or more additional linkers through, for example, the epsilon amino group of the side chain.

Linking groups may comprise one or more alkyl groups such as methyl, ethyl, propyl, butyl, etc. groups, alkoxy groups, alkenyl groups, alkynyl groups or amino group substituted by alkyl groups, cycloalkyl groups, polycyclic groups, aryl groups, polyaryl groups, substituted aryl groups, heterocyclic groups, and substituted heterocyclic groups. Linking groups may also comprise polyethoxy aminoacids such as AEA ((2-amino) ethoxy acetic acid) or AEEA ([2-(2-amino)ethoxy)]ethoxy acetic acid). In one embodiment, the one or more linking groups is selected from the group consisting of polyglycine, polylysine, AEEA, AEA and OA (8-amino octanoic acid). In certain embodiments, the linking group is a peptide having a labile chemical bond which is cleavable by an enzyme or which is cleaved under acidic conditions.

In certain embodiments, the linking group may be selected from linking groups including an amino group and a carboxy group including, but not limited to, AEA, AEEA and OA. In certain embodiments, the linking group may be a polymer of AEA having a length of 1 to 100 atoms. In certain embodiments, the linking group may be an AEEA polymer having a length of 1 to 100 atoms. In certain embodiments, the linking group may be an OA polymer having a length of 1 to 100 atoms. Illustrative examples of linking groups include monomers, dimers, trimers, tetramers, pentamers, sixmers, septamers, octamers, nonamer, decamer, undecamers, dodecamers of glycine, lysine, glutamate, isoleucine, or arginine residues, AEA, AEEA or OA (e.g., an OA dimer (-OA-OA-) or an OA trimer (-OA-OA-OA-), or any combination thereof (e.g., any combination of Glyₙ, Lysₙ, OA, AEA, or AEEA, wherein n=1, 2, 3, 4, 5, 6, 7, 8, 9, 10, 11, or 12). In some embodiments, the linking group has a single lysine. The single lysine can be modified to be linked to a reactive group directly or via one or more linking groups. For example, the K group may be linked to the reactive group or one or more additional linkers through, for example, the epsilon amino group of the side chain. Examples of such linkers including a single lysine have, for example, the sequence of (monomer)ₐK(monomer)_{b}, K(monomer)c, and (monomer)_{d}K, where a, b, c and d are each an integer greater than or equal to one, for example, one, two, three, four, five, six, seven, eight, nine, ten, eleven, twelve or more. In some examples, a and b may be the same, whereas in other examples a and b are not the same. For example, a may be three and b may be four to provide a linking group having the following sequence: (OA)ₙK(OA)ₙ, (OA)ₙK(AEA)ₙ, (G)ₙK(OA)ₙ, (OA)ₙK(G)ₙ, wherein n=1, 2, 3, 4, 5, 6, 7, 8, 9, 10, 11, or 12. In the case where the reactive group is located on the linker between the two TMPs, the total length between the two TMPs will not typically exceed 100 atoms. The attachments to the linker group are not considered to be part of the linker between the two TMPs.

The linking group can include any combinations of the aforesaid biocompatible polymers. For example, a polyglycine linker can be combined with one or more monomers of AEA, AEEA or OA in any configuration. In one embodiment, the polyglycine linker is attached to one or more monomers of AEA, AEEA or OA at either the N- or the C-terminal end of the first or last glycine residue in the polyglycine linker. Alternatively one or more monomers of AEA, AEEA or OA are inserted in between glycine residues in the polyglycine linker.

In particular embodiments, the reactive group (for example, MPA, GMBA, NHS, sulfo-NHS, MBS or GMBS), is attached to the peptide through one or more linking groups, including, for example, a polyglycine linker, a polyglycine-lysine linker, AEEA, AEA, or OA, or any combination thereof. In certain embodiments in which the reactive group is attached to the peptide through more than one linking group, each linking group can be independently selected from the group consisting typically of, polyglycine, polylysine, AEA, AEEA, and OA. In embodiments, the number of linking groups (e.g., monomeric polymer units) is from 1 to 2, 1 to 3, 1 to 4, 1 to 5, 1 to 6, 1 to 7, 1 to 8, 1 to 9, 1 to 10, 1 to 11, 1 to 12. Where there is more than one linking group, the linking groups can be the same or different linking groups. For example, any combination of one of polyglycine, polylysine, AEA, AEEA, and/or OA can be used in any order. In one embodiment, the reactive group, typically MPA, is attached to the peptide via 1, 2, 3, 4, 5, 6, 7, 8, 9, 10, 11, or 12 polyglycine, polylysine, AEA, AEEA or OA linking groups which are arranged in tandem. In another embodiment, the reactive group, typically MPA, is attached to the peptide via 1, 2, 3, 4, 5, 6, 7, 8, 9, 10, 11, or 12 polyglycine, polylysine, AEA, AEEA or OA linking groups which are arranged in a branched configuration.

Typically, the peptide and serum protein (e.g., albumin) of the conjugates of the invention are present in the conjugate in a 1:1 molar ratio, or an approximately 1:1 molar ratio. In a preferred embodiment, the peptide and serum protein are present in the conjugate in a 1:1 molar ratio, or an approximately 1:1 molar ratio, and the peptide is attached to a residue of the reactive group, optionally through a linking group, at substantially only one site on the peptide and the residue of the reactive group is attached to the serum protein at substantially only one site on the serum protein. In the foregoing context, attached at “substantially only one site” means that at least 70%, 75%, 80%, 85%, 90%, 95% or 99% of the attachments are at a single site.

The serum protein is albumin, and the single site of attachment of the reactive group to the albumin can be the thiol of cysteine 34 of albumin (e.g., via a maleimide linkage). In a specific embodiment, the reactive group is a single MPA reactive group attached to the peptide, optionally through a linking group, at a single defined amino acid and the MPA is covalently attached to albumin at substantially a single amino acid residue of albumin, typically cysteine 34. In particular embodiments, the reactive group is a single MPA reactive group attached to the peptide, optionally through a linking group, at the epsilon amino group of a lysine and a residue of the MPA is covalently attached to albumin at substantially a single amino acid residue of albumin, typically cysteine 34. In one embodiment, the albumin is recombinant human albumin. In certain embodiments, the reactive group, typically MPA, is attached to the peptide through one or more linking groups, typically AEEA, AEA, or OA (8-amino octanoic acid or octanoyl). In certain examples of embodiments in which the reactive group, typically MPA, is attached to the peptide through more than one linking group, each linking group can be independently selected from the group consisting typically of, polyglycine, polylysine, AEA, AEEA, and OA. Typically the number of linking groups is from 1 to 6. Where there is more than one linking group, the linking groups can be identical or different linking groups. In one embodiment, the reactive group, typically MPA, is attached to the peptide via 1, 2, 3, 4, 5 or 6 AEEA linking groups which are arranged in tandem. In another embodiment, the reactive group, typically MPA, is attached to the peptide via 1, 2, 3, 4, 5 or 6 OA linking groups which are arranged in tandem.

The present invention also provides methods for covalently attaching a modified peptide to a serum protein or peptide, thereby forming a conjugate of the invention. In one embodiment, a modified peptide covalently attached to a serum protein is prepared in vivo by administration of a modified peptide directly to a subject such that a residue of the reactive group of the peptide forms a covalent bond with one or more serum proteins in vivo. Such modified peptides for in vivo conjugation with serum proteins are referred to herein as “prodrugs.” The prodrug may optionally contain one or more protecting groups which prevent the reactive group from forming covalent bonds in vitro. Typically, the protecting group of a prodrug according to the invention is labile in vivo, so that the reactive group is released from its protection and is free to covalently bond to a serum protein in vivo after administration to the subject.

In another embodiment, a modified peptide comprising a serum protein is prepared in vitro (ex vivo) by covalently attaching the modified peptide to the serum protein in vitro such that a residue of the reactive group of the peptide forms a covalent bond with the serum protein. In one embodiment, the serum protein is autologous to the subject. In a specific embodiment, the serum protein is isolated from the subject. In certain embodiments, the isolated serum protein from the subject is purified from other proteins present in the blood and/or from blood cells before it is covalently attached to the modified peptide. In accordance with this embodiment, the resulting conjugate is administered to the subject from which the serum protein was isolated, or to an autologous subject. In another embodiment, the serum protein is a recombinant serum protein. Typically, the serum protein is recombinant albumin, most typically the serum protein is recombinant human albumin.

In a preferred embodiment, a conjugate of the invention is formed by contacting a modified peptide comprising a maleimido group with a thiol-containing serum protein, typically albumin, under conditions comprising a pH of between 6.5 and 7.4, thereby typically forming a stable thioether linkage which cannot be cleaved under physiological conditions. In certain preferred embodiments, the serum protein is recombinant human albumin.

In one embodiment, the modified peptide is amidated at its C-terminal end. In another embodiment, the modified peptide is not amidated at its C-terminal end. A modified peptide, conjugate or compound of the invention can also comprise such an amidated peptide.

In one embodiment, the modified peptide is acylated at its N-terminal end. In another embodiment, the modified peptide is not acylated at its N-terminal end. A modified peptide, conjugate, compound of the invention can also comprise such an acylated peptide.

The present invention provides conjugates having improved pharmacokinetic properties compared to unconjugated peptides. The pharmacokinetic properties of a peptide include, for example, its rate of absorption and excretion, its tissue distribution profile, its rate of metabolism and its toxicity. Typically, the conjugates of the invention have a decreased rate of excretion and/or an increased circulating half-life in vivo, compared to unconjugated peptides. Pharmacokinetic properties are discussed in more detail in Section 4.3.

The modified peptides and conjugates of the invention typically retain at least some of the biological activity of the corresponding unmodified or unconjugated peptides. In certain embodiments a modified peptide or conjugate of the invention has improved biological activity compared to the corresponding unmodified or unconjugated peptide. Biological activity includes in vitro activity, for example, receptor binding, e.g., to a thrombopoietin receptor such as cMP-1 or a member of its family of subtypes. Biological activity also includes in vivo activity, for example, the ability to promote megakaryopoiesis or thrombopoiesis.

### Exemplary Modified Thrombopoietin Peptides

In certain embodiments, the modified thrombopoietin peptides of the invention include a generic formula as follows:

(LL_{A})ₖ-(LL_{B})ₘ-(LL_{C})ₙ-(TMP₁)-(LL_{D})_{z}-(TMP₂)-(LL_{E})ₚ-(LL_{F})_{q}-(LL_{G})ᵣ

In certain embodiments, z is an integer greater than or equal to one, and the sum of k, m, n, p, q and r is an integer that is zero or greater, more particularly, the sum of k, m, n, p, q and r is an integer greater than or equal to one. Thus, where k is one, m, n, p, q and r may be zero. Where m is 1, then k, n, p, q and r may be zero. Where n is one, then k, m, p, q and r may be zero. Similarly, where p is one, then k, m, n, q and r may be zero. Where q is one, then k, m, n, p and r may be zero. Where r is one, then k, m, n, p, and q may be zero. In some instances, the sum of k, m, n, p, q and r is at least two, three, four, five, six, seven, eight, nine, ten, fifteen, twenty or more with any one or more of k, m, n, p, q and r being absent. In one embodiment, the sum of k, m, n, p, q and r is greater than or equal to one if z is an integer greater than one, for example, two or more. In certain instances, z may be one, two, three, four, five, six, seven, eight, nine, ten or more. In a particular embodiment where z is one, the sum of k, m, n, p, q and r may be zero as detailed further below.

The exact groups used for the linking groups LL_{A}, LL_{B}, LL_{C}, LL_{D}, LL_{E}, LL_{F} and LL_{G} may vary. As used herein, the term “linking group” is used interchangeably in certain instances with the term “linker.” In addition, any two or more of the various linking groups LL_{A}, LL_{B}, LL_{C}, LL_{D}, LL_{E}, LL_{F} and LL_{G} may be the same or different. In some embodiments, one of the linking groups may be replaced with a reactive group, which may be used, for example, to conjugate the composition to a protein or peptide. Exemplary configurations of compositions that include one or more linking groups and a reactive group are described herein. Illustrative linking groups include those described herein, for example, those having a length of 1 to 100 atoms. Specific linking groups include, but are not limited to, polyglycine, polyglutamate, polyisoleucine, polyarginine or other suitable linking groups including two or more amino acids. In some examples, the amino acid linking groups can include at least two, three, four, five, six, eight, nine, ten, eleven or twelve amino acid residues, for example, glycine or lysine residues.

In certain embodiments, the linking group may be selected from linking groups including an amino group and a carboxy group including, but not limited to, AEA ((2-amino) ethoxy acetic acid), AEEA ([2-(2-amino)ethoxy)]ethoxy acetic acid) and OA (8-amino octanoic acid, also called 8-amino caprylic acid, of formula NH₂—(CH₂)₇—COOH). In certain embodiments, the linking group may be a polymer of AEA having a length of 1 to 100 atoms. In certain embodiments, the linking group may be an AEEA polymer having a length of 1 to 100 atoms. In certain embodiments, the linking group may be an OA polymer having a length of 1 to 100 atoms. Illustrative examples of linking groups include monomers, dimers, trimers, tetramers or pentamers of AEA, AEEA or OA (e.g., an OA dimer (-OA-OA-) or an OA trimer (-OA-OA-OA-)). The linking group can also include any combinations of the aforesaid biocompatible polymers. For example, a polyglycine linker can be combined with one or more monomers of AEA, AEEA or OA in any configuration. In one embodiment, the polyglycine linker is attached to one or more monomers of AEA, AEEA or OA at either the N- or the C-terminal end of the first or last glycine residue in the polyglycine linker. Alternatively one or more monomers of AEA, AEEA or OA are inserted in between glycine residues in the polyglycine linker. In other embodiments, the linking group is a peptide having a labile chemical bond which is cleavable by an enzyme or which is cleavable under acidic conditions. In examples, where a polyglycine is used as a linker, the polyglycine may include a single lysine to provide a free epsilon amino group capable of reacting with another linker or with a protein. Examples of such a polyglycine that includes a single lysine have, for example, an amino acid sequence of (G)ₐK(G)_{b}, K(G)c, and (G)_{d}K. where a, b, c and d are each an integer greater than or equal to one, for example, one, two, three, four, five, six, seven, eight, nine, ten, eleven, twelve or more. In some examples, a and b may be the same, whereas in other examples a and b are not the same. For example, a may be three and b may be four to provide a linking group having an amino acid sequence of GGGKGGGG (SEQ ID NO:163).

In some examples, the single lysine within the polyglycine linker may itself be linked to additional linking groups. For example, the lysine group may be linked to one or more additional linkers through, for example, the epsilon amino group of the side chain. A generic structure may be represented, for example, as

K-(LL_{H})_{w}-(LL_{I})ₓ-(LL_{J})_{y}

In the formula above, the sum of w, x, and y is an integer greater than or equal to zero, for example, one, two, three, four, five, six, seven, eight, nine, ten or more. Where LL_{H} is bound to the lysine group through the lysine's epsilon amino group, an amide bond may form from reaction of the amino side chain and a carboxyl group of the LL_{H} linking group.

In some examples, one or more of the linking groups may be replaced with, or substituted for, a reactive group. For example, the linking group may be replaced with a maleimide-containing group, preferably gamma-maleimide-butylamide (GMBA) or maleimido propionic acid (MPA). The reactive maleimide group may be used to conjugate the composition to a peptide, protein or other species as discussed further below. The resulting product (for example, a TMP conjugated to a protein through the linking group) may result in a residue of the reactive group conjugating the TMP component of the composition with the protein component of the composition. For example, a reactive group residue may result from reaction of the reactive group with an amino acid of the protein or peptide.

In examples where z is eight, m is one, n is five, and k, p, q, and r are each zero, the composition may have a formula of

(LL_{B})-(LL_{C})₅-(TMP₁)-(LL_{D})₈-(TMP₂).

A specific representation of this formula is MPA-(Gly)₅-(TMP₁)-(Gly)₈-(TMP₂). In this specific formulation, the linker LL_{B} has been replaced with the reactive group MPA, LL_{C} and LL_{D} are each glycine, TMP₁ and TMP₂ are each a peptide having a sequence as shown below to provide (MPA)-(Gly)₅-(TMP₁)-(Gly)₈-(TMP₂). TMP₁ and TMP₂ are each a peptide having a sequence of IEGPTLRQWLAARA or a TMP variant described herein.

Another specific composition of the generic formula where z is eight, p is two, q is one, r is one and each of k, m and n is zero is

(TMP₁)-(LL_{D})₈-(TMP₂)-(LL_{E})₂-(LL_{F})-(LL_{G}).

A specific representation of this formula is TMP₁-(Gly)₈-TMP₂—OA-OA-K-MPA, where LL_{D} is glycine, LL_{E} is an 8-aminooctanoyl linking group, LL_{F} is lysine and LL_{G} has been replaced with the reactive group MPA linked to the lysine through the lysine's epsilon amino group. TMP₁ and TMP₂ are each a peptide having a sequence of IEGPTLRQWLAARA or other TMP variant described herein. In some examples, the MPA shown as LL_{G} may instead, be removed and LL_{G} may be a linking group (or a different reactive group) that can react with the C-terminus of the lysine group. Illustrative such linking groups are capable of forming an amide bond between the C-terminus of the lysine and an amino group of the linker. One such different reactive group is a derivatized MPA that includes a primary amino group.

Another specific composition of the generic formula where k, m and n are zero, z is eight, p is one, q is two and r is one provides (TMP₁)-(LL_{D})₈-(TMP₂)-(LL_{E})-(LL_{F})₂-(LL_{G}). In one embodiment, LL_{D} is glycine, LL_{E} is lysine, LL_{F} is OA and LL_{G} has been replaced with the reactive group MPA to provide a composition having a formula of (TMP₁)-(Gly)₈-(TMP₂)-K-OA-OA-MPA with TMP₁ and TMP₂ each being a peptide having a sequence of IEGPTLRQWLAARA or a TMP variant described herein.

Another specific composition of the generic formula where z is three, m is one, n is two and k, p, q and r are each zero is (LL_{B})-(LL_{C})₂-(TMP₁)-(LL_{D})₃-(TMP₂). In one embodiment, LL_{B} has been replaced with MPA, LL_{C} and LL_{D} may each be OA with one of the OA groups of LL_{C} being bound to the N-terminal amino group of Ile (or other N-terminal amino acid residue) of TMP₁ and LL_{D} may be OA to provide (MPA)-(OA)₂-(TMP)₁-(OA)₃-(TMP₂). TMP₁ and TMP₂ are each a peptide having a sequence of IEGPTLRQWLAARA or a TMP variant described herein.

Another specific composition of the generic formula where z is one and each of k, m, n, p, q and r is zero provides (TMP₁)-(LL_{D})-(TMP₂). In this specific composition, LL_{D} may be a polyglycine that includes a lysine group such as, for example, (G)ₐK(G)_{b}, K(G)_{c}, and (G)_{d}K. where a, b, c and d are each an integer greater than or equal to one, for example, one, two, three, four, five, six, seven, eight, nine, ten or more. The lysine itself may be linked to a linking group through its epsilon amino group to provide K-(LL_{H})_{w}-(LL_{I})ₓ-(LL_{J})_{y}. Thus, the formula may be represented as:

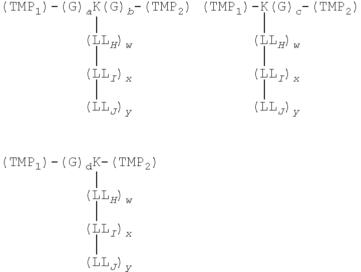

In the formulae above, LL_{H}, LL_{I}, and LL_{J} may be any of the linkers discussed above, and LL_{H}, LL_{I}, and LL_{J} may be the same or may be different. Any of LL_{H}, LL₁, and LL_{J} may be replaced with a reactive group such as, for example, MPA. In certain embodiments, the sum of w, x, and y is an integer greater than or equal to one, for example, one, two, three, four, five, six, seven, eight, nine, ten or more. In one embodiment where LL_{D} is (G)ₐK(G)_{b}, where a is three and b is four, LL_{H} is OA, LL_{I}, has been replaced with MPA, w is two, x is one and y is zero, the following composition is provided

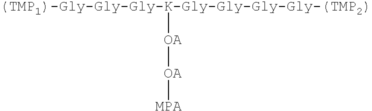

with the epsilon amino group of the lysine coupled to the carboxyl group of one of the 8-aminooctanoyl (OA) groups. Each of TMP₁ and TMP₂ is a peptide having a sequence of IEGPTLRQWLAARA or a TMP variant described herein.

In certain embodiments, any one or more of the compositions listed above may be coupled to a peptide or protein using the reactive group of the composition or directly to the peptide or protein. For example, a composition as shown above that includes an MPA linking group may react with a thiol group of a cysteine in a protein to conjugate the composition to the protein through a residue of the MPA group.

In some examples, the composition may be Protein-(LL_{B})-(LL_{C})₅-(TMP₁)-(LL_{D})₈-(TMP₂), where the protein is conjugated through the LL_{B} group, which may be for example, a residue of an MPA group or a GMBA group. In other examples, the composition may be (TMP₁)-(LL_{D})₈-(TMP₂)-(LL_{E})₂-(LL_{F})-(LL_{G})-Protein, where the protein is conjugated through the LL_{G} group, which may be for example, a residue of an MPA group or a GMBA group. In certain embodiments, the composition may be (TMP₁)-(LL_{D})₈-(TMP₂)-(LL_{E})-(LL_{F})₂-(LL_{G})-Protein, where the protein is conjugated through the LL_{G} group, which may be for example, a residue of an MPA group or a GMBA group. In other embodiments, the composition may be Protein-(LL_{B})-(LL_{C})₂-(TMP₁)-(LL_{D})₃-(TMP₂), where the protein is conjugated through the LL_{B} group, which may be for example, a residue of an MPA group or a GMBA group. In some embodiments, the composition may be (TMP₁)-[(LL_{D})-Protein]-(TMP₂), where the protein is conjugated through the LL_{D} group and the TMP₁ and TMP₂ groups are coupled to each other through the LL_{D} linker. In some examples, LL_{D} may be a (G)ₐK(G)_{b} group such that a composition of (TMP₁)-[(G)ₐ-[K-Protein]-(G)_{b}]-(TMP₂) is provided, where the protein is conjugated through the side chain of the lysine (for example, through a residue of an MPA group or a GMBA group) and the glycine residues are coupled to each other through the lysine. In certain examples, LL_{D} may be a K(G)_{c} group such that a composition of (TMP₁)-[K-Protein]-(G)_{c}]-(TMP₂) is provided, where the protein is conjugated through the side chain of the lysine for example, through a residue of an MPA group or a GMBA group. In additional examples, LL_{D} may be a (G)_{d}K group such that a composition of (TMP₁)-[(G)d-[K-Protein]]-(TMP₂) is provided, where the protein is conjugated through the side chain of the lysine for example, through a residue of an MPA group or a GMBA group.

In certain embodiments, one or more of the linking groups may be replaced with a lysine. For example, the lysine can be substituted for one or more of linkers LL_{A}, LL_{B}, LL_{C}, LL_{D}, LL_{E}, LL_{F}, LL_{G}, LL_{H}, LL_{K}, LL₁, LL_{L}, LL_{M} or other linking groups. Where a lysine does replace a linking group, the lysine may react with a reactive group to conjugate the composition to a peptide or protein. Thus, by replacing different linking groups with the lysine, the lysine group may be moved up or down the composition relative to the position of one or more of the TMP groups, e.g., may be moved more atoms away from one or both of the TMP groups or may be moved fewer atoms from one or more of the TMP groups. By selecting the particular residue where the lysine is placed, the particular site of attachment of the composition to a peptide or protein may be varied

For illustrative purposes only, certain resulting conjugates are shown below for certain compositions described above where one of the linking groups has been replaced with a reactive group, such as, for example, a maleimido group. Additional suitable compositions may be linked to a desired protein or peptide in a similar manner or through a different reactive group than MPA or directly to the composition. In the structures below, X is the particular group of the protein that reacts with the reactive linking group. For example, X may be sulfur, oxygen, nitrogen or other groups that can add to the double bond of the maleimide group. In some examples, the X group is the reside of an amino acid side chain that forms a covalent bond with an atom of the reactive group. In certain examples, the X group may be provided by reaction of the cysteine thiol group with the double bond of the maleimido group.

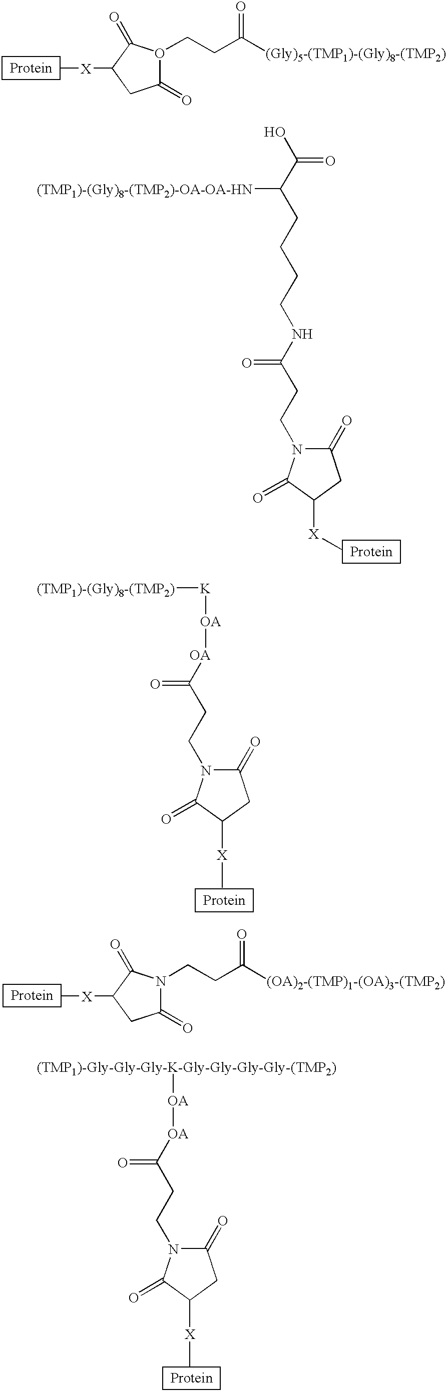

In certain embodiments, more than two TMP groups may be present in the composition. For example, two, three, four or more TMP groups may be present. In some examples, the composition disclosed herein may be dimers in that the entire composition repeats itself twice. The dimers may be linked through one or more groups that include a scissile bond such that cleavage of the scissile bond can provide two units of the composition. Illustrative labile bonds include but are not limited to, arginine-lysine peptide bonds, which may be cleaved enzymatically using trypsin, aromatic amino acid-tryptophan peptide bonds (F-W and Y-W bonds), which may be cleaved using chymotrypsin, an arginine peptide bond, which may be cleaved with clostripain, an aspartate or glutamate peptide bond, which may be cleaved with Staphylococcal protease, a methionine peptide bond, which may be cleaved with cyanogen bromide, an asparagine-glycine peptide bond, which may be cleaved with hydroxylamine, and aspartate-proline peptide bonds, which may be cleaved at pH. 2.5, 40° C. Additional labile bonds and reagents or conditions for cleaving them are also possible.

Illustrative dimeric compositions include, but are not limited to,

(MPA)-(Gly)₅-(TMP₁)-(Gly)₈-(TMP₂)-(Gly)₅-(TMP₁)-(Gly)₈-(TMP₂),

TMP₁-(Gly)₈-TMP₂—OA-OA-K-TMP₁-(Gly)₈-TMP₂—OA-OA-K-MPA,

(TMP₁)-(Gly)₈-(TMP₂)-K-OA-OA-(TMP₁)-(Gly)₈-(TMP₂)-K-OA-OA-MPA,

and

(MPA)-(OA)₂-(TMP)₁-(OA)₃-(TMP₂)-(MPA)-(OA)₂-(TMP)₁-(OA)₃-(TMP₂).

In other embodiments, there may be more than a single type of linking group between the TMP groups. For example, the TMP groups may be separated by linking groups as shown in the following formula

(TMP₁)-(LL_{K})_{f}-(LL_{L})_{g}-(LL_{M})ₕ-(TMP₂)

Where LL_{K}, LL_{L} and LL_{M} are each a linking group (which may be the same or may be different) and f, g and h are each an integer that is zero, one, two, three, four, five, six, seven, eight, nine, ten or more. In some examples, the sum of f, g, and h is zero, one, two, three, four, five, six, seven, eight, nine, ten or more such that the linking groups LL_{K}, LL_{L} and LL_{M} may be absent or such that any one or more of the linking groups LL_{K}, LL_{L} and LL_{M} may be present. Thus, where the TMP groups include two or more linking groups between them, a generic formula shown below can be provided.

(LL_{A})ₖ-(LL_{B})ₘ-(LL_{C})ₙ-(TMP₁)-(LL_{K})_{f}-(LL_{L})_{g}-(LL_{M})ₕ-(TMP₂)-(LL_{E})ₚ-(LL_{F})_{q}-(LL_{G})ᵣ

In the formula above, the various linking groups and subscripts thereof may be any of those species discussed herein for a particular linking group and/or subscript thereof. For example, any one or more of the linking groups may be AEA, AEEA, OA or the like. The subscripts k, m, n, f, h, p, q and r may each by zero, one, two, three, four, five, six, seven, eight, nine, ten or more. In some examples, the sum of a particular group or subset of subscripts may be, for example, any one of those described herein.

Specific exemplary conjugates of the invention have the following structures:

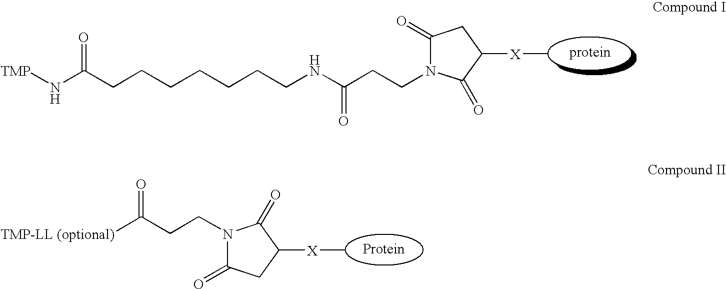

wherein TMP is a thrombopoietin peptide of the invention and X is S, O, or NH of an amino acid of said protein. In certain embodiments, said protein is albumin. In certain embodiments, said protein is albumin and X is S of Cys 34 of said albumin. Albumin of the conjugate can be any albumin as described above. The protein can be linked to any site of the thrombopoietin peptide, including, for example, the N-terminus, the C-terminus or an amino acid side chain. Compound I depicts coupling via a linking group. Compound II shows an example of direct coupling to MPA having an optional linker (LL). In specific embodiments, the protein is linked to the epsilon amino group of a lysine side chain of the peptide. When the reactive group is coupled to the protein using a reactive group that includes a double bond between two carbons, the protein may add to the double bond of the reactive group at either carbon.

In certain embodiments, the conjugate has the following structure:

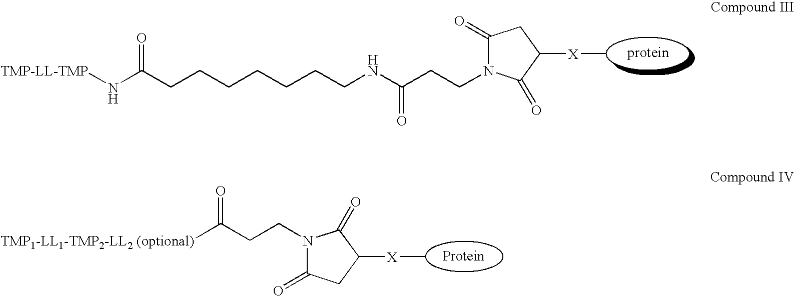

wherein TMP is a thrombopoietin peptide of the invention, LL, LL₁ and LL₂ are each a linker, and X is S, O, or NH of an amino acid of said protein. In certain embodiments, said protein is albumin. In certain embodiments, said protein is albumin and X is S of Cys 34 of said albumin. Albumin of the conjugate can be any albumin as described above. Said protein can be linked to any site of the thrombopoietin peptide, including, for example, the N-terminus, the C-terminus or an amino acid side chain. Compound III depicts coupling via a linking group. Compound IV shows an example of direct coupling to MPA having an optional linker (LL). In specific embodiments, the protein is linked to the epsilon amino group of a lysine side chain of the peptide. The thrombopoietin peptides can be linked via any linkage apparent to those of skill in the art. For example, they can be linked via a C-terminal to C-terminal linkage, an N-terminal to N-terminal linkage, or an N-terminal to C-terminal linkage. In certain embodiments, they can be linked via one or more side chains, e.g., side chain hydroxyls, thiols, carboxyls or amides. The side chain can be linked to an N-terminus, a C-terminus, or another side chain wherein appropriate. When the reactive group is coupled to the protein using a reactive group that includes a double bond between two carbons, the protein may add to the double bond of the reactive group at either carbon.

### Thrombopoietin Peptide Derivatives

In certain embodiments, the thrombopoietin peptide in the modified peptides, compounds or conjugates of the invention is a derivative of a thrombopoietin peptide of a SEQ ID NO. A derivative of a thrombopoietin peptide of a particular SEQ ID NO has one or more amino acid substitutions, deletions, and/or additions that are not present in the particular SEQ ID NO. Typically, the number of amino acids substituted, deleted, or added is 1, 2, 3, 4, 5, 6, 7, 8, 9, or 10 amino acids. In one embodiment, such a derivative contains one or more amino acid deletions, substitutions, or additions at the amino and/or carboxy terminal end of the peptide. In another embodiment, such a derivative contains one or more amino acid deletions, substitutions, or additions at any residue within the length of the peptide.

In certain embodiments, the amino acid substitutions may be conservative or non-conservative amino acid substitutions. Conservative amino acid substitutions are made on the basis of similarity in polarity, charge, solubility, hydrophobicity, hydrophilicity, and/or the amphipathic nature of the amino acid residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for another class.

In certain embodiments, an amino acid substitution may be a substitution with a non-classical amino acid or chemical amino acid analog. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In certain embodiments, a derivative of a thrombopoietin peptide shares an overall sequence homology with the peptide of at least 75%, at least 85%, or at least 95%. Percent homology in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology. In certain embodiments, a derivative of a thrombopoietin peptide is characterized by its percent sequence identity or percent sequence similarity with the peptide. Sequence homology, including percentages of sequence identity and similarity, are determined using sequence alignment techniques well-known in the art, typically computer algorithms designed for this purpose, using the default parameters of said computer algorithms or the software packages containing them.

Non-limiting examples of computer algorithms and software packages incorporating such algorithms include the following. The BLAST family of programs exemplify a preferred, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another preferred example is the algorithm of Myers and Miller (1988 CABIOS 4:11-17) which is incorporated into the ALIGN program (version 2.0) and is available as part of the GCG sequence alignment software package. Also preferred is the FASTA program (Pearson W. R. and Lipman D. J., Proc. Nat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length.

In certain embodiments, a derivative of a thrombopoietin peptide shares a primary amino acid sequence homology over the entire length of the sequence, without gaps, of at least 55%, at least 65%, at least 75%, or at least 85% with the peptide. In a preferred embodiment, a modified thrombopoietin peptide of the invention shares a primary amino acid sequence homology over the entire length of the sequence, without gaps, of at least 90% or at least 95% with the peptide.

In a preferred embodiment, the percent identity or similarity is determined by determining the number of identical (for percent identity) or conserved (for percent similarity) amino acids over a region of amino acids, which region is equal to the total length of the shortest of the two peptides being compared (or the total length of both, if the sequence of both are identical in size). In another embodiment, percent identity or similarity is determined using a BLAST algorithm, with default parameters.

### Reactive Groups

A reactive group is a chemical group which, after conjugation to a thrombopoietin peptide, is capable of forming a covalent bond with a serum protein or peptide. A residue of a reactive group is the chemical moiety of a reactive group after the reactive group forms the covalent bond with a serum protein or peptide to form the conjugates of the invention. Thus, in the context of a conjugate, “a residue of a reactive group” has already reacted with the serum protein to form a covalent bond with the serum protein or peptide.

A reactive group may form a covalent bond with a serum protein or peptide, for example, through an amino, hydroxyl, carboxyl, or thiol group of the serum protein or peptide. Typically, a reactive group forms only one covalent bond with the serum protein or peptide, most typically at a specifically defined amino acid of the serum protein or peptide. Such reactive groups are covalently attached to a thrombopoietin peptide of interest to form a modified thrombopoietin peptide. Reactive groups are typically stable in an aqueous environment. In specific embodiments, the reactive group comprises a carboxy, phosphoryl, or acyl group. In certain embodiments, the acyl group is an ester or a mixed anhydride, or an imidate. In certain embodiments, the reactive group comprises a phenolic group, a thiol ester, an alkyl ester, or a phosphate ester.

In one embodiment, the reactive group comprises an α-, β-unsaturated carbonyl Michael acceptor. In one embodiment, the reactive group comprises a maleimide or a maleimido group which forms a covalent bond with a thiol group of a serum protein. In one embodiment, the reactive group comprises a thiol group which forms a covalent bond with an imidate or thioester group of a serum protein. In one embodiment, the reactive group comprises a carboxy, phosphoryl, or acyl group which forms a covalent bond with an amino group of a serum protein.

In one embodiment, the reactive group comprises a succinimidyl or a maleimido group. A succinimide containing reactive group may be referred to herein as a succinimidyl group. A maleimide containing reactive group may be referred to herein as a maleimido group. In a specific embodiment, the reactive group is selected from the group consisting of N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), gamma-maleimide-butrylamide (GMBA), and maleimidopropionic acid (MPA).

In one embodiment, the reactive group comprises an N-hydroxysuccinimide (NHS) ester which forms a covalent bond with the epsilon amine of a lysine residue of a serum protein. Other reactive groups which may be utilized are described in U.S. Pat. No. 5,612,034, which is hereby incorporated by reference.

In one embodiment, the reactive group comprises a maleimide containing group such as GMBA or MPA which forms a covalent bond with a thiol group of a serum protein. In a specific embodiment, the reactive group forms a covalent bond with the thiol group of a cysteine of the serum protein. Typically, the reactive group forms a covalent bond with cysteine 34 (Cys34) of albumin.

### Maleimide Containing Reactive Groups

A modified peptide comprising a maleimide containing reactive group offers several advantages. First, maleimide-peptides are generally stable in aqueous solutions and in the presence of free amines, providing a modified peptide product with a longer shelf-life. Since maleimide containing groups will only react with free thiols, protecting groups are generally not necessary to prevent the maleimide-peptide from reacting with itself or with other peptides in solution. The increased stability of the maleimide-peptide facilitates further purification steps, which may include chromatographic steps such as high pressure liquid chromatography (“HPLC”). The inclusion of further purification steps is preferred for the preparation of highly purified products suitable for in vivo use.

Another advantage of a modified peptide comprising a maleimide reactive group is the preferential reaction of the maleimide with thiol groups on the serum proteins rather than with amino groups. This means that a modified peptide comprising a maleimide group will covalently bond to fewer different serum proteins in vivo because thiol groups are less abundant on serum proteins than amino groups. Specifically, a modified peptide of the invention comprising a maleimide group is most likely to form a covalent bond in vivo with either albumin or IgG, because both of these serum proteins contain one or more accessible free thiol groups.

A further advantage of a modified peptide comprising a maleimide reactive group is the ability to form well-defined conjugates with the serum protein, albumin, which is the most abundant serum protein. Since albumin contains only a single free thiol, the thiol of Cys34, the maleimide group of the modified peptide preferentially binds to albumin only at Cys34, giving conjugates of approximately a 1:1 molar ratio of peptide to albumin. Thus, a distinct advantage of the maleimide peptides of the invention is the ability to reproducibly form conjugates with albumin in an approximately 1:1 molar ratio with the site of attachment to albumin being a specifically defined amino acid residue on albumin, i.e., Cys 34. Other techniques, such as glutaraldehyde, DCC, EDC and other chemical activations of, for example, free amines, lack this selectivity and give instead heterogeneous populations of conjugates in which the peptide may be covalently bound to the serum protein at any one of a number of possible amino acid residues, e.g., lysine residues, and wherein the serum protein may conjugate to multiple peptides. For example, albumin contains 52 lysine residues, 25-30 of which are located on the surface of the protein and accessible for conjugation. A technique which utilizes chemical activation of amines, or a modified peptide comprising a reactive group which preferentially binds to amines, can form conjugates with albumin at any one of these available lysine residues. The result is multiple conjugation products, some containing 0, 1, 2 or more peptides per albumin, and each having peptides randomly coupled to any one of the 25-30 available lysine residues. Given the numerous combinations possible, characterization of the exact composition and nature of each batch becomes difficult, and batch-to-batch reproducibility is all but impossible, making such conjugates less desirable as therapeutic agents.

In one embodiment, a single reactive group is covalently attached at a defined site of the modified peptide. In one embodiment of the conjugate, a single reactive group is covalently attached at a defined site of the modified peptide and the reactive group is covalently attached to a single defined site of albumin, typically to the thiol group of amino acid residue Cys34 of albumin. Typically, the reactive group of a modified peptide or conjugate of the invention comprises a maleimide group and forms peptide:albumin conjugates of approximately a 1:1 molar ratio. In certain embodiments, a 1:1 molar ratio of peptide to serum protein is preferred over higher ratios because a 1:1 molar ratio provides better biological activity than higher ratios (see e.g., Stehle et al. 1997 Anti-Cancer Drugs 8:677-685, incorporated herein in its entirety).

The manner of modifying thrombopoietin peptide with a reactive group for conjugation to a serum protein will vary widely, depending upon the nature of the various elements comprising the thrombopoietin peptide. The synthetic procedures will be selected so as to be simple, provide for high yields, and allow for a highly purified product. Normally, the chemically reactive group will be created at the last stage of thrombopoietin peptide synthesis, for example, with a carboxyl group, esterification to form an active ester. Specific methods for the production of modified peptides are described in U.S. Pat. No. 6,329,336, 6,849,714 or 6,887,849, the contents of which are hereby incorporated by reference in their entireties.

In an embodiment where the modified peptide is administered to a subject, the modified peptide of the invention comprising a maleimide group will form a conjugate in vivo with albumin preferentially over other thiol-containing serum proteins. The preferential conjugation to albumin is likely because the free thiol of albumin Cys34 has increased reactivity relative to free thiols of other serum proteins. This is due in part to the low pKa of albumin Cys34, making the ionized form of this cysteine predominant under normal physiological conditions and thereby increasing the reactivity of this residue. Albumin Cys34 is also more reactive due in part to its location in a crevice close to the surface of one loop of region V of albumin, making it very accessible.

### Albumin

Any albumin or variant thereof known to those of skill in the art may be used to form a conjugate of the present invention. In some embodiments, the albumin may be albumin isolated from serum and purified for use in the formation of a conjugate. The albumin may be any mammalian albumin known to those of skill in the art, including but not limited to mouse, rat, rabbit, guinea pig, dog, cat, sheep, bovine, ovine, equine, or human albumin. In some embodiments, the albumin is human albumin.

Albumin can be provided from a number of sources, such as purified from blood or expressed by recombinant techniques including in e.g. recombinant yeast or bacteria. In certain embodiments, the albumin is recombinant albumin. The recombinant albumin may be any mammalian albumin known to those of skill in the art, including but not limited to mouse, rat, rabbit, guinea pig, dog, cat, sheep, bovine, ovine, equine, or human albumin. In one embodiment, the recombinant albumin is recombinant human albumin, in particular, recombinant human serum albumin (rHSA).

Human serum albumin (HSA) is responsible for a significant proportion of the osmotic pressure of serum and also functions as a carrier of endogenous and exogenous ligands. In its mature form, HSA is a non-glycosylated monomeric protein of 585 amino acids, corresponding to a molecular weight of about 66 kD. Its globular structure is maintained by 17 disulfide bridges which create a sequential series of 9 double loops. See Brown, J. R., Albumin Structure, Function and Uses, Rosenoer, V. M. et al. (eds), Pergamon Press, Oxford (1977), the content of which is hereby incorporated by reference in its entirety. In a specific embodiment, the invention provides conjugates formed with the mature form of albumin.

In some embodiments, conjugates of the present invention comprise an albumin precursor. Human albumin is synthesized in liver hepatocytes and then secreted in the blood stream. This synthesis leads, in a first instance, to a precursor, prepro-HSA, which comprises a signal sequence of 18 amino acids directing the nascent polypeptide into the secretory pathway. In a specific embodiment, the invention provides conjugates formed with an albumin precursor.

In certain embodiments, conjugates of the invention comprise molecular variants of albumin, for example as those described in WO 2005/058958, the content of which is incorporated by references herein in its entirety. A recombinant human serum albumin variant is commercially available from New Century Pharma (Huntsville, Ala.) under the name Albagen™.

Variants of albumin may include natural variants resulting from the polymorphism of albumin in the human population. More than 30 apparently different genetic variants of human serum albumin have been identified by electrophoretic analysis under various conditions. See e.g., Weitkamp et al., Ann. Hum. Genet., 36(4):381-92 (1973); Weitkamp, Isr. J. Med. Sci., 9(9):1238-48 (1973); Fine et al., Biomedicine, 25(8):291-4 (1976); Fine et al., Rev. Fr. Transfus. Immunohematol., 25(2):149-63. (1982); Rochu et al., Rev. Fr. Transfus. Immunohematol. 31(5):725-33 (1988); Arai et al., Proc. Natl. Acad. Sci. U.S.A 86(2): 434-8 (1989), the contents of which are hereby incorporated by reference in their entireties. In a specific embodiment, the invention provides conjugates formed with molecular variants of albumin.

In some embodiments, conjugates of the invention comprise derivatives of albumin which share substantial homology with albumin. For instance, conjugates may be formed with an albumin homologue having an amino acid sequence at least 75%, at least 80%, at least 85%, more typically at least 90%, and most typically at least 95%, the same as that of albumin. In certain embodiments, the albumin homologue comprises a free cysteine. In certain embodiments, the albumin homologue comprises a single free cysteine. In some embodiments, the albumin homologue comprises a free cysteine 34.

In some embodiments, conjugates of the invention comprise structural derivatives of albumin. Structural derivatives of albumin may include proteins or peptides which possess an albumin-type activity, for example, a functional fragment of albumin. In some embodiments, the derivative is an antigenic determinant of albumin, i.e., a portion of a polypeptide that can be recognized by an anti-albumin antibody. In some embodiments, the recombinant albumin may be any protein with a high plasma half-life which may be obtained by modification of a gene encoding human serum albumin. By way of example and not limitation, the recombinant albumin may contain insertions or deletions in the trace metal binding region of albumin, such that binding of trace metals, e.g., nickel and/or copper is reduced or eliminated, as described in U.S. Pat. No. 6,787,636, the content of which is incorporated by reference in its entirety. Reduced trace metal binding by albumin may be advantageous for reducing the likelihood of an allergic reaction to the trace metal in the subject being treated with the albumin composition.

Structural derivatives of albumin may be generated using any method known to those of skill in the art, including but not limited to, oligonucleotide-mediated (site-directed) mutagenesis, alanine scanning, and polymerase chain reaction (PCR) mutagenesis. Site-directed mutagenesis (see Carter, Biochem. J. 237:1-7 (1986); Zoller and Smith, Methods Enzymol. 154:329-50 (1987)), cassette mutagenesis, restriction selection mutagenesis (Wells et al., Gene 34:315-323 (1985)) or other known techniques can be performed on cloned albumin-encoding DNA to produce albumin variant DNA or sequences which encode structural derivatives of albumin (Ausubel et al., Current Protocols In Molecular Biology, John Wiley and Sons, New York (current edition); Sambrook et al., Molecular Cloning, A Laboratory Manual, 3d. ed., Cold Spring Harbor Laboratory Press, Cold Spring Harbor, N.Y. (2001), the contents of which are hereby incorporated by reference in their entireties.

In certain embodiments, albumin derivatives include any macromolecule by in vitro modification of the albumin protein with a plasma half-life higher than native albumin. In some embodiments, the albumin is modified with one or more fatty acids. In some embodiments, the albumin is modified with one or more metal ions. In some embodiments, the albumin is modified with one or more small molecules having high affinity to albumin. In some embodiments, the albumin is modified with one or more sugars, including but not limited to, glucose, lactose, mannose, and galactose.

Albumin, albumin variants or derivatives for use in forming a conjugate of the present invention may be obtained using methods or materials known to those of skill in the art. For instance, albumin can be obtained from a commercial source, e.g., as RECOMBUMIN® (Novozymes Inc., Nottingham, UK); PLASBUMIN® (Talecris Biotherapeutics, Research Triangle Park, N.C.); ALBAGEN®, (New Century Pharmaceuticals, Huntsville, Ala.); human albumin (Cortex-Biochem, San Leandro, Calif.), human serum albumin, ZLB Behring (King of Prussia, Pa.), or ALBREC® (Mistubishi Pharma, Japan).

Typically, the formation of a conjugate with albumin or an albumin variant or derivative is done ex vivo.

### Peptide Synthesis

The peptides of the invention, including peptide linker groups, may be synthesized by standard methods of solid or solution phase peptide chemistry. A summary of the solid phase techniques may be found in Stewart and Young (1963) Solid Phase Peptide Synthesis, W. H. Freeman Co. (San Francisco), and Meienhofer (1973) Hormonal Proteins and Peptides, Academic Press (New York). For classical solution synthesis see Schroder and Lupke, The Peptides, Vol. 1, Academic Press (New York).

In general, these methods comprise the sequential addition of one or more amino acids or suitably protected amino acids to a growing peptide chain. Normally, either the amino or carboxyl group of the first amino acid is protected by a suitable protecting group. The protected amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth. After all the desired amino acids have been linked in the proper sequence, any remaining protecting groups (and any solid support) are removed sequentially or concurrently to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

In certain embodiments, the peptides of the invention are synthesized with amino- and carboxy-protecting groups for use as pro-drugs. Protecting groups are chemical moieties which block a reactive group on the peptide to prevent undesirable reactions. In one embodiment, a modified peptide of the invention is synthesized with one or more protecting groups that are designed to be cleaved in vivo, thereby exposing the reactive group or groups of the modified peptide to serum proteins after administration of the peptide to a subject. Further examples of protecting groups are provided in the sections that follow.

### Solid Phase Synthesis

The synthesis of the peptide derivatives disclosed herein was performed as follows. Chemicals were purchased from commercial suppliers. The synthesis was performed using an automated solid-phase procedure on a Symphony Peptide Synthesizer with manual intervention during the generation of the DAC™ moiety. The synthesis was performed on Fmoc-protected Ramage amide linker resin using Fmoc-protected amino acids methodology. Coupling of the amino acids was achieved by using O-benzotriazol-1-yl-N,N,N′,N′-tetramethyl-uronium hexafluorophosphate (HBTU) as activator in N,N-dimethylformamide (DMF) solution and diisopropylethylamine (DIEA) as base. The Fmoc protective group was removed using 20% piperidine/DMF. When needed, a Boc-protected amino acid was used at the N-terminus in order to generate the free N_{α}-terminus after the peptide is cleaved from resin. When needed, the selective deprotection of the Lys (Aloc) group was performed manually and accomplished by treating the resin with a solution of 3 eq of Pd(PPh₃)₄ dissolved in 5 mL of C₆H₆CHCl₃ (1:1): 2.5% NMM (v:v): 5% AcOH (v:v) for 2 h. The resin is then washed with CHCl₃ (6×5 mL), 20% AcOH in DCM (6×5 mL), DCM (6×5 mL), and DMF (6×5 mL). When needed, the synthesis was re-automated for the addition of the Fmoc-8-Amino-OctanoicAcid (Fmoc-OA-OH) followed by the 3-maleimidopropionic acid (MPA). Between every coupling, the resin was washed 3 times with N,N-dimethylformamide (DMF) and 3 times with isopropanol (ⁱPrOH). The different peptides were cleaved from the resin using 85% TFA/5% triisopropyl-silane (TIPS)/5% thioanisole and 5% phenol, followed by precipitation by dry-ice cold Et₂O prior to purification.

### HPLC Purification

The peptide crude products were purified by preparative reversed phased HPLC using a Varian (Rainin) preparative binary HPLC system: gradient elution of 32-42% B (0.045% TFA in H₂O (A) and 0.045% TFA in CH₃CN (B)) over 180 min at 9.5 mL/min using a Phenomenex Luna 10μ phenyl-hexyl, 21 mm×250 mm column and UV detector (Varian Dynamax UVD II) at λ214 and 254 nm to afford the desired peptides (i.e., native and DAC™) in >95% purity, as determined by RP-HPLC.

### Characterization Protocols (HPLC:LC/MS)

The purified products were analysed by reversed phased HPLC using a Varian (Rainin) preparative binary HPLC system: gradient elution of 20-60% B (0.045% TFA in H₂O (A) and 0.045% TFA in CH₃CN (B)) over 20 min at 1.0 mL/min using a Phenomenex Luna 10μ phenyl-hexyl, 4.6 mm×250 mm column and UV detector (Varian Dynamax UVD II) at λ214 and 254 nm. In addition, the mass of the purified products were measured by an Agilent 1100 serie LC/EI-MS with a gradient elution of 20-60% B (0.045% TFA in H₂O (A) and 0.045% TFA in CH₃CN (B)) over 30 min at 1.0 mL/min using a Phenomenex Luna 10μ phenyl-hexyl, 4.6 mm×250 mm column and UV detector at λ214 followed by mass detection on the 1100 serie EI-MS. The product presented a mass that was in accordance with the theoritical calculation.

### Amino-Protecting Groups

The term “amino-protecting group” refers to those groups intended to protect the amino-terminal end of an amino acid or peptide or to protect the amino group of an amino acid or peptide against undesirable reactions. Commonly used amino-protecting groups are disclosed in Greene (1981) Protective Groups in Organic Synthesis (John Wiley & Sons, New York), which is hereby incorporated by reference. Additionally, protecting groups can be used which are readily cleaved in vivo, for example, by enzymatic hydrolysis, thereby exposing the amino group for reaction with serum proteins in vivo.

Amino-protecting groups comprise lower alkanoyl groups such as formyl, acetyl (“Ac”), propionyl, pivaloyl, and t-butylacetyl; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, and 4-nitrobenzoyl; sulfonyl groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, αα-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2, -trichloroethoxycarbonyl, phenoxycarbonyl, 4-nitrophenoxycarbonyl, fluorenyl-9-Page 50 of 82 methoxycarbonyl, cyclopentyloxycarbonyl, adamantyloxycarbonyl, cyclohexyloxycarbonyl, and phenylthiocarbonyl; arylalkyl groups such as benzyl, triphenylmethyl, benzyloxymethyl, 9-fluorenylmethyloxycarbonyl (Fmoc) and silyl groups such as trimethylsilyl.

### Carboxy Protecting Groups

The term “carboxy protecting group” refers to a carboxylic acid protecting ester or amide group employed to block or protect the carboxylic acid functionality. Carboxy protecting groups are disclosed in Greene, “Protective Groups in Organic Synthesis” pp. 152-186 (1981), which is hereby incorporated by reference. Additionally, a carboxy protecting group can be used as a pro-drug whereby the carboxy protecting group can be readily cleaved in vivo, for example by enzymatic hydrolysis, thereby exposing the carboxy group for reaction with serum proteins in vivo. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated by reference.

Representative carboxy protecting groups are C₁-C₈ lower alkyl (e.g., methyl, ethyl or t-butyl); arylalkyl such as phenethyl or benzyl and substituted derivatives thereof such as alkoxybenzyl or nitrobenzyl groups; arylalkenyl such as phenylethenyl; aryl and substituted derivatives thereof such as 5-indanyl; dialkylaminoalkyl such as dimethylaminoethyl); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, and propionyloxymethyl; cycloalkanoyloxyalkyl groups such as cyclopropylcarbonyloxymethyl, cyclobutylcarbonyloxymethyl, cyclopentylcarbonyloxymethyl, and cyclohexylcarbonyloxymethyl; aroyloxyalkyls such as benzoyloxymethyl and benzoyloxyethyl; arylalkylcarbonyloxyalkyls such as benzylcarbonyloxymethyl and 2-benzylcarbonyloxyethyl; alkoxycarbonylalkyl or cycloalkyloxycarbonylalkyl such as methoxycarbonylmethyl, cyclohexyloxycarbonylmethyl, and 1-methoxycarbonyl-1-ethyl; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, and 1-cyclohexyloxycarbonyloxy-1-ethyl; aryloxycarbonyloxyalkyl such as 2-(phenoxycarbonyloxy)ethyl, and 2-(5-indanyloxycarbonyloxy)ethyl; alkoxyalkylcarbonyloxyalkyl such as 2-(1-methoxy-2-methylpropan-2-oyloxy)ethyl; arylalkyloxycarbonyloxyalkyl such as 2-(benzyloxycarbonyloxy)ethyl; arylalkenyloxycarbonyloxyalkyl such as 2-(3-phenylpropen-2-yloxycarbonyloxy)ethyl; alkoxycarbonylaminoalkyl such as t-butyloxycarbonylaminomethyl; alkylaminocarbonylaminoalkyl such as methylaminocarbonylaminomethyl; alkanoylaminoalkyl such as acetylaminomethyl; heterocycliccarbonyloxyalkyl such as 4-methylpiperazinylcarbonyloxymethyl; dialkylaminocarbonylalkyl such as dimethylaminocarbonylmethyl, diethylaminocarbonylmethyl; (5-(loweralkyl)-2-oxo-1,3-dioxolen4-yl)alkyl such as (5-t-butyl-2-oxo-1,3-dioxolen-4-yl)methyl; and (5-phenyl-2-oxo-1,3-dioxolen-4-yl)alkyl such as (5-phenyl-2-oxo-1,3-dioxolen-4-yl)methyl.

Preferred carboxy-protected peptides of the invention are peptides wherein the protected carboxy group is a lower alkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, and phenylethyl ester or an alkanoyloxyalkyl, cycloalkanoyloxyalkyl, aroyloxyalkyl or an arylalkylcarbonyloxyalkyl ester. Preferred amide carboxy protecting groups are lower alkylaminocarbonyl groups. For example, aspartic acid may be protected at the α-C-terminal by an acid labile group (e.g., t-butyl) and protected at the β-C-terminal by a hydrogenation labile group (e.g., benzyl) then deprotected selectively during synthesis.

### Pharmacokinetic Properties

The compounds, modified peptides, and/or conjugates of the invention typically exhibit improved pharmacokinetic properties compared to unmodified or unconjugated peptides. For example, the peptides of the invention typically exhibit improved profiles of one or more of the following properties: absorption, distribution, metabolism, excretion and toxicity. These properties are determined using standard pharmacological and profiling assays available in the art. Typically, a compound (comprising a modified peptide with a reactive group as described herein) or conjugate of the invention for therapeutic use has one or more of the following characteristics: a slower rate of elimination compared to the corresponding unmodified or unconjugated peptide, an increased circulating half-life compared to the corresponding unmodified or unconjugated peptide, and solubility in aqueous solution. Typically, a compound or conjugate of the invention for therapeutic use has increased bioavailability upon administration compared to the unmodified or unconjugated peptide.

### Therapeutic Uses

The compounds, modified peptides, and/or conjugates of the invention can be used in the prevention or treatment of a disease or disorder in a subject. The term “treatment” refers to a therapeutic measure. The treatment may be administered to a subject having a medical disorder or who ultimately may acquire the disorder, in order to cure, delay, reduce the severity of, and/or ameliorate one or more symptoms of a disorder or recurring disorder, or in order to prolong the survival of a subject beyond that expected in the absence of such treatment. The term “prophylaxis” or “prevention” refers to a preventive measure. The treatment may be administered to a subject having a medical disorder or who ultimately may acquire the disorder, in order to prevent, delay onset of one or more symptoms of a disorder or recurring disorder, or in order to prolong the survival of a subject beyond that expected in the absence of such treatment.

The term “effective amount” refers to a dosage or amount of the modified TPO peptide or conjugate that is sufficient to modulate one or more activities of TPO to, for example, ameliorate clinical symptoms or achieve a desired biological outcome, e.g., increased platelet production. The “effective amount” may be administered prophylactically or therapeutically, that is, upon single or multiple dose administration to a subject (such as a human patient), it can delay onset of or prevent, or treat or cure, at least one symptom of a disorder or recurring disorder, or prolonging the survival of the subject beyond that expected in the absence of such treatment.

Typically, the disease or disorder is one for which administration of a thrombopoietin agonist, e.g., a thrombopoietin peptide, to a subject has shown some efficacy. In certain embodiments, the compounds or conjugates of the invention are useful for megakaryocytopoiesis. In certain embodiments, the compounds or conjugates of the invention are useful for thrombopoiesis. In certain embodiments, the compounds or conjugates of the invention are useful for the treatment of prevention of any condition or disorder where increased megakaryocytopoiesis is useful. In certain embodiments, the compounds or conjugates of the invention are useful for the treatment of prevention of any condition or disorder where increased thrombopoiesis is useful.

In certain embodiments, the compounds or conjugates of the invention can be used in the prevention or treatment of hematological disorders, including but not limited to platelet disorders and thrombocytopenia. In certain embodiments, the thrombocytopenia is associated with bone marrow transfusion, radiation therapy, or chemotherapy. In certain embodiments, the thrombocytopenia is immune thrombocytopenic purpura. In certain embodiments, the thrombocytopenia is caused by myelodysplastic syndrome or leukemia. In certain embodiments, the thrombocytopenia is caused by a disease or condition damaging the liver such as hepatitis or cirrhosis. In certain embodiments, the thrombocytopenia is caused by infectious diseases such as HIV infection. In certain embodiments, the thrombocytopenia is caused by trauma or surgery such as liver transplantation or cardiopulmonary bypass. In certain embodiments, the thrombocytopenia is caused by substances such as heparin.

In certain embodiments, the present invention provides methods of harvesting platelets from a subject. In the methods, a compound or conjugate of the invention is used to increase platelet production in the subject. In certain embodiments, platelet production can be monitored by any technique known to those of skill in the art. In the methods, platelets are harvested from the subject when appropriate according to the judgment of one of skill in the art. The platelets can be harvested by any technique without limitation. Suitable techniques are within the skill of those in the art.

The activity of the compounds of the present invention can be evaluated either in vitro or in vivo in one of the numerous models described in McDonald, 1992, Am. J. of Pediatric Hematology/Oncology 14:8-21, which is incorporated herein by reference.

### Compositions and Methods of Administering

The invention also provides compositions (including pharmaceutical compositions) comprising a compound or conjugate of the invention, and a pharmaceutically acceptable carrier, excipient, or diluent. In certain embodiments, a composition of the invention may also contain minor amounts of wetting or emulsifying agents, or pH buffering agents. In a specific embodiment, the pharmaceutical composition is pharmaceutically acceptable for administration to a human. In certain embodiments, the pharmaceutical composition comprises a therapeutically or prophylactically effective amount of a compound or conjugate of the invention. The amount of a compound or conjugate of the invention that will be therapeutically or prophylactically effective can be determined by standard clinical techniques. Exemplary effective amounts are described in more detail herein. In certain embodiments, a composition of the invention may also contain a stabilizer. A stabilizer is a compound that reduces the rate of chemical degradation of the modified peptide of the composition. Suitable stabilizers include, but are not limited to, antioxidants, such as ascorbic acid, pH buffers, or salt buffers.

The pharmaceutical compositions of the invention can be in any form suitable for administration to a subject, typically a human subject. In certain embodiments, the compositions of the invention take the form of solutions, suspensions, emulsion, tablets, pills, capsules, powders, and sustained-release formulations. The compositions may also be in particular unit dosage forms. Examples of unit dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

In a specific embodiment, the subject is a mammal such as a cow, horse, sheep, pig, fowl, cat, dog, mouse, rat, rabbit, or guinea pig. In one embodiment, the subject is a human. Typically, the pharmaceutical composition is suitable for veterinary and/or human administration. In accordance with this embodiment, the term “pharmaceutically acceptable” means approved by a regulatory agency of the Federal or a state government or listed in the U.S. Pharmacopeia or other generally recognized pharmacopeia for use in animals, and more particularly for use in humans.

Suitable pharmaceutical carriers for use in the compositions of the invention are sterile liquids, such as water and oils, including those of petroleum, animal, vegetable or synthetic origin. In a specific embodiment, the oil is peanut oil, soybean oil, mineral oil, or sesame oil. Water is a preferred carrier when the pharmaceutical composition is administered intravenously. Saline solutions and aqueous dextrose and glycerol solutions can also be employed as liquid carriers, particularly for injectable solutions. Further examples of suitable pharmaceutical carriers are known in the art, e.g., as described by E. W. Martin in Remington's Pharmaceutical Sciences (1990) 18th ed. (Mack Publishing, Easton Pa.).

Suitable excipients for use in the compositions of the invention include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. Whether a particular excipient is suitable for incorporation into a pharmaceutical composition depends on a variety of factors well known in the art including, but not limited to, the route of administration and the specific active ingredients in the composition.

In certain embodiments, a composition of the invention is an anhydrous composition. Anhydrous compositions can be prepared using anhydrous or low moisture containing ingredients and low moisture or low humidity conditions. Compositions comprising modified peptides having a primary or secondary amine are typically anhydrous if substantial contact with moisture and/or humidity during manufacturing, packaging, and/or storage is expected. An anhydrous composition should be prepared and stored such that its anhydrous nature is maintained. Accordingly, anhydrous compositions are typically packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

### Formulations

Pharmaceutical compositions comprising the compounds or conjugates of the invention, or their pharmaceutically acceptable salts and solvates, are formulated to be compatible with the intended route of administration. The formulations are typically for subcutaneous administration, but can be for administration by other means such as by inhalation or insufflation (either through the mouth or the nose), intradermal, oral, buccal, parenteral, vaginal, or rectal. Typically, the compositions are also formulated to provide increased chemical stability of the compound during storage and transportation. The formulations may be lyophilized or liquid formulations.

In one embodiment, the compounds or conjugates of the invention are formulated for oral administration. Oral formulations can include standard carriers such as pharmaceutical grades of mannitol, lactose, starch, magnesium stearate, sodium saccharine, cellulose, magnesium carbonate, etc. In another embodiment, the compounds or conjugates of the invention are formulated for injection. In one embodiment, the compounds or conjugates of the invention are sterile lyophilized formulations, substantially free of contaminating cellular material, chemicals, virus, or toxins. In a particular embodiment, the compounds or conjugates of the invention are formulated in liquid form. In another particular embodiment, formulations for injection are provided in sterile single dosage containers. In a particular embodiment, formulations for injection are provided in sterile single dosage containers. The formulations may or may not contain an added preservative. Liquid formulations may take such forms as suspensions, solutions or emulsions in oily or aqueous vehicles, and may contain formulation agents such as suspending, stabilizing and/or dispersing agents.

### Methods of Administration

A compound or conjugate of the invention, or a pharmaceutically acceptable salt thereof, is typically administered as a component of a composition that optionally comprises a pharmaceutically acceptable vehicle. The compound or conjugate is typically administered subcutaneously. Another method of administration is via intravenous injection of the compound or conjugate.

In certain embodiments the compound or conjugate of the invention is administered by any other convenient route, for example, by infusion or bolus injection, or by absorption through epithelial or mucocutaneous linings (e.g., oral mucosa, rectal, and intestinal mucosa). Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In most instances, administration will result in the release of the compound or conjugate of the invention into the bloodstream.

In one embodiment, a compound of the invention comprising a maleimide containing reactive group is administered to a subject in a controlled manner such that 80-90% of the administered peptide forms conjugates with albumin and less than 5% forms conjugates with IgG. Such specific conjugation is for in vivo use as it permits an accurate calculation of the estimated half-life of the administered peptide.

In another embodiment, a compound of the invention comprising a maleimide containing reactive group is added to blood, serum, or a saline solution containing serum albumin and/or IgG, under conditions permitting the formation of a covalent bond between the reactive group of the peptide and the albumin and/or IgG, resulting in the formation of a conjugate of the peptide with the albumin or IgG. In a further embodiment, the blood, serum, or saline solution containing the conjugate is administered to a subject.

In certain embodiments, the compounds or conjugates of the invention are administered in combination with one or more other biologically active agents as part of a treatment regimen. In certain embodiments, the compounds or conjugates are administered prior to, concurrently with, or subsequent to the administration of the one or more other biologically active agents. In one embodiment, the one or more other biologically active agents is administered in the same pharmaceutical composition with a compound or conjugate of the invention. In another embodiment, the one or more other biologically active agents is administered in a separate pharmaceutical composition with a compound or conjugate of the invention. In accordance with this embodiment, the one or more other biologically active agents may be administered to the subject by the same or different routes of administration as those used to administer the compound or conjugate of the invention.

In certain embodiments, the compounds or conjugates of the invention are administered in combination with one or more cancer therapy such as bone marrow transfusion, radiation therapy, or chemotherapy.

The term “in combination with” includes the administration of two therapeutic agents either simultaneously, concurrently or sequentially with no specific time limits. In one embodiment, both agents are present in a subject at the same time or exert their biological or therapeutic effect at the same time. In one embodiment, the two therapeutic agents are in the same composition or unit dosage form. In another embodiment, the two therapeutic agents are in separate compositions or unit dosage forms. In certain embodiments, the compound or conjugate of the invention is administered prior to the cancer therapy. In certain embodiments, the compound or conjugate of the invention is administered one week prior to, three days prior to, two days prior to, one day prior to, twelve hours prior to or six hours prior to the cancer therapy.

### Dosages and Frequency

The amount of a compound or conjugate of the invention, or the amount of a composition comprising the compound or conjugate, that will be effective in the prevention or treatment of a disease or disorder can be determined by standard clinical techniques. In vitro or in vivo assays may optionally be employed to help identify optimal dosage ranges. The precise dose to be employed will also depend, e.g., on the route of administration, the type of invention, and the seriousness of the disease or disorder, and should be decided according to the judgment of the practitioner and each patient's circumstances.

Exemplary doses of the compounds or conjugates of the invention, or of compositions (typically pharmaceutical compositions) comprising same, include milligram or microgram or picogram amounts per kilogram of the subject (e.g., about 10 picogram per kilogram to about 500 milligrams per kilogram, about 100 picograms per kilogram to about 5 milligrams per kilogram, or about 1 microgram per kilogram to about 50 micrograms per kilogram). In specific embodiments, a daily dose is at least 1 μg, 5 μg, 10 μg, 100 μg, 250 μg, 500 μg, 750 μg, 1 mg, 5 mg, 5 mg, 10 mg, 25 mg, 50 mg, 75 mg, or at least 100 mg.

In one embodiment, the dosage is a concentration of 0.001 to 5000 mM, 0.01 to 500 mM, 0.1 to 300 mM and 1 mM to 100 mM. In another embodiment, the dosage is a concentration of at least 0.1 μM, 1 μM, 5 μM, at least 10 μM, at least 50 μM, at least 100 μM, at least 500 μM, at least 1 mM, at least 5 mM, at least 10 mM, at least 50 mM, at least 100 mM, or at least 500 mM.

In one embodiment, the dosage is a concentration of 0.001 to 5000 mM, 0.001 to 500 mM, 0.1 to 300 mM and 1 mM to 100 mM. In another embodiment, the dosage is a concentration of at least 0.1 μM, 1 μM, 5 μM, at least 10 μM, at least 50 μM, at least 100 μM, at least 500 μM, at least 1 mM, at least 5 mM, at least 10 mM, at least 50 mM, at least 100 mM, or at least 500 mM.

In a specific embodiment, the dosage is 0.01 μg/kg or more, typically 0.1 μg/kg or more, 0.25 μg/kg or more, 0.5 μg/kg or more, 1 μg/kg or more, 2 μg/kg or more, 3 μg/kg or more, 4 μg/kg or more, 5 μg/kg or more, 6 μg/kg or more, 7 μg/kg or more, 8 μg/kg or more, 9 μg/kg or more, or 10 μg/kg or more, 25 μg/kg or more, typically 50 μg/kg or more, 100 μg/kg or more, 250 μg/kg or more, 500 μg/kg or more, 1 mg/kg or more, 5 mg/kg or more, 6 mg/kg or more, 7 mg/kg or more, 8 mg/kg or more, 9 mg/kg or more, or 10 mg/kg or more of a patient's body weight.

In another embodiment, the dosage is a unit dose of 0.1 μg, 1 μg, typically 5 μg, 10 μg, 50 μg, 100 μg, 250 μg, 500 μg, 750 μg, 1 mg, 5 mg, 10 mg, 50 mg, 100 mg, 150 mg, 200 mg, 250 mg, 300 mg, 350 mg, 400 mg, 500 mg, 550 mg, 600 mg, 650 mg, 700 mg, 750 mg, 800 mg or more.

In another embodiment, the dosage is a unit dose that ranges from about 0.1 μg to about 1 g, typically about 1 μg to about 500 mg, about 100 μg to about 300 mg, about 1 mg to about 100 mg, 1 mg to about 500 mg, about 10 mg to about 800 mg, about 500 mg to about 1000 mg, or about 5 mg to about 1000 mg.

In certain embodiments, suitable dosage ranges for oral administration are about 0.001 milligram to about 5 grams of a compound or conjugate of the invention of the invention, or a pharmaceutically acceptable salt thereof, per kilogram body weight per day. In specific embodiments of the invention, the oral dose is about 0.01 milligram to about 100 milligrams per kilogram body weight per day, typically about 0.1 milligram to about 75 milligrams per kilogram body weight per day, and more typically about 0.5 milligram to 5 milligrams per kilogram body weight per day. The dosage amounts described herein refer to total amounts administered; that is, if more than one compound or conjugate is administered, then, in some embodiments, the dosages correspond to the total amount administered. In a specific embodiment, oral compositions contain about 10% to about 95% a peptide of the invention by weight.

In some embodiments, suitable dosage ranges for intravenous (i.v.) administration are about 1 picogram to about 100 milligrams per kilogram body weight per day, about 1 microgram to about 10 milligrams per kilogram body weight per day, and about 10 microgram to about 1 milligrams per kilogram body weight per day. In some embodiments, suitable dosage ranges for intranasal administration are about 0.01 pg/kg body weight per day to about 1 mg/kg body weight per day. Suppositories generally contain about 0.01 microgram to about 50 milligrams of a compound of the invention per kilogram body weight per day and comprise active ingredient in the range of about 0.1% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 500 milligrams per kilogram of body weight per day. Suitable doses for topical administration are in the range of about 0.001 milligram to about 50 milligrams, depending on the area of administration. Effective doses may be extrapolated from dose-response curves derived from in vitro or animal model test systems. Such animal models and systems are well known in the art.

In another embodiment, a subject is administered one or more doses of a prophylactically or therapeutically effective amount of a compound or conjugate of the invention, or of a composition comprising same, wherein the prophylactically or therapeutically effective amount is not the same for each dose. In another embodiment, a subject is administered one or more doses of a prophylactically or therapeutically effective amount of a compound or conjugate of the invention, or of a composition comprising same, wherein the dose of a prophylactically or therapeutically effective amount administered to said subject is increased by, e.g., 0.001 μg/kg, 0.002 μg/kg, 0.005 μg/kg, 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses. In another embodiment, a subject is administered one or more doses of a prophylactically or therapeutically effective amount of a compound or conjugate of the invention, wherein the dose is decreased by, e.g., 0.001 μg/kg, 0.002 μg/kg, 0.005 μg/kg, 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses.

The above-described administration schedules are provided for illustrative purposes only and should not be considered limiting. A person of ordinary skill in the art will readily understand that all doses are within the scope of the invention.

### Kits

The invention provides a pharmaceutical pack or kit comprising one or more containers filled with one or more of the compounds or conjugates of the invention for the prevention or treatment of a disease or disorder, or one or more symptoms thereof.

In one embodiment, the kit comprises a compound or conjugate of the invention, in one or more containers. In one embodiment, the kit comprises a pharmaceutical composition of the invention, in one or more containers. In one embodiment, the kit optionally contains one or more other biologically active agents useful for the prevention or treatment of a disease or disorder, or one or more symptoms thereof, in one or more other containers. In one embodiment, the kit comprises a compound or conjugate of the invention in lyophilized form. In accordance with this embodiment, the kit may optionally contain a container of sterile solution suitable for reconstituting the lyophilized compound or conjugate. In one embodiment, the kit comprises a unit dosage form of a compound or conjugate of the invention in one or more containers.

Typically, a kit further comprises instructions for preventing or treating the disease or disorder, as well as side effects and dosage information for specific methods of administration. Optionally associated with such container(s) can be a notice in the form prescribed by a governmental agency regulating the manufacture, use or sale of pharmaceuticals or biological products, which notice reflects approval by the agency of manufacture, use or sale for human administration.

The Examples that follow are set forth to aid in the understanding of the inventions but are not intended to, and should not be construed to, limit its scope in any way.

### EXAMPLES

The following Examples disclose general synthetic methods (Example 1), and provide examples of specific examples of modified thrombopoietin peptides of the invention (Examples 2 and 3). Example 4 describes the results of in vivo testing the modified thrombopoietin peptides.

### Example 1

### Synthesis and Purification of Peptide Derivatives

### General Peptide Solid Phase Protocol

Chemicals were purchased from commercial suppliers and were used as received. The synthesis of all the peptide derivatives disclosed herein was performed using an automated solid-phase procedure on a Symphony Peptide Synthesizer with manual intervention during the generation of the DAC™ moiety. The synthesis was performed on Fmoc-protected Ramage amide linker resin using Fmoc-protected amino acids methodology. Coupling of the amino acids was achieved by using O-benzotriazol-1-yl-N,N,N′,N′-tetramethyl-uronium hexafluorophosphate (HBTU) as activator in N,N-dimethylformamide (DMF) solution and diisopropylethylamine (DIEA) as base. The Fmoc protective group was removed using 20% piperidine/DMF. When needed, a Boc-protected amino acid was used at the N-terminus in order to generate the free N_{α}-terminus after the peptide is cleaved from resin. When needed, the selective deprotection of the Lys (Aloc) group was performed manually and accomplished by treating the resin with a solution of 3 eq of Pd(PPh₃)₄ dissolved in 5 mL of C₆H₆CHCl₃ (1:1): 2.5% NMM (v:v): 5% AcOH (v:v) for 2 h. The resin is then washed with CHCl₃ (6×5 mL), 20% AcOH in DCM (6×5 mL), DCM (6×5 mL), and DMF (6×5 mL). When needed, the synthesis was re-automated for the addition of the Fmoc-8-Amino-OctanoicAcid (Fmoc-OA-OH) followed by the 3-maleimidopropionic acid (MPA). Between every coupling, the resin was washed 3 times with N,N-dimethylformamide (DMF) and 3 times with isopropanol (ⁱPrOH). The different peptides were cleaved from the resin using 85% TFA/5% triisopropyl-silane (TIPS)/5% thioanisole and 5% phenol, followed by precipitation by dry-ice cold Et₂O prior to purification.

### General HPLC Purification Protocol

The peptide crude products were purified by preparative reversed phased HPLC using a Varian (Rainin) preparative binary HPLC system: gradient elution of 32-42% B (0.045% TFA in H₂O (A) and 0.045% TFA in CH₃CN (B)) over 180 min at 9.5 mL/min using a Phenomenex Luna 10μ phenyl-hexyl, 21 mm×250 mm column and UV detector (Varian Dynamax UVD II) at λ214 and 254 nm to afford the desired peptides (i.e., native and DAC™) in >95% purity, as determined by RP-HPLC.

### General Characterization Protocols (HPLC: LC/MS)

The purified products were analysed by reversed phased HPLC using a Varian (Rainin) preparative binary HPLC system: gradient elution of 20-60% B (0.045% TFA in H₂O (A) and 0.045% TFA in CH₃CN (B)) over 20 min at 1.0 mL/min using a Phenomenex Luna 10μ phenyl-hexyl, 4.6 mm×250 mm column and UV detector (Varian Dynamax UVD II) at λ214 and 254 nm. In addition, the mass of the purified products were measured by an Agilent 1100 serie LC/EI-MS with a gradient elution of 20-60% B (0.045% TFA in H₂O (A) and 0.045% TFA in CH₃CN (B)) over 30 min at 1.0 mL/min using a Phenomenex Luna 10μ phenyl-hexyl, 4.6 mm×250 mm column and UV detector at λ214 followed by mass detection on the 1100 serie EI-MS. The producted presented a mass that was in accordance with the theoritical calculation.

The modified thrombopoietin peptides may optionally be amidated at the C-termini or acylated at the N-termini. The term “reactive group” as used in the tables below refers to a reactive group, e.g., as described herein, particularly as described herein. The thrombopoietin sequences provided in Tables 1-3 include the linking groups. For example, SEQ ID NO:25 is a sequence of a thrombopoietin peptide (ARAALWQRLTPGEI (SEQ ID NO: 24)) plus a linking group of twenty glycines.

These peptides can be covalently attached to a serum protein such as albumin in vivo, following administration to a subject, or in vitro (ex vivo), where the conjugate is to be administered to the subject. Typically, the peptides are covalently attached to a serum protein, typically albumin, in vitro, to form a conjugate of the invention by following routine experiments as described herein.

### Example 2

### Examples of Modified Thrombopoietin Peptides

### ARAALWQRLTPGEI (SEQ ID NO:24)

TABLE 1
Modified Thrombopoietin Peptides Containing a Linking Group

| | Linker | |
|---|---|---|
| Sequence | Abbreviation | SEQ ID NO |
| (reactive group)-GGGGGGGGGGGGGGGGGGGG- | G₂₀ (SEQ ID | SEQ ID NO:25 |
| ARAALWQRLTPGEI | NO: 129) | |
| (reactive group)-GGGGGGGGGGGGGGGGGGG- | G₁₉ (SEQ ID | SEQ ID NO:26 |
| ARAALWQRLTPGEI | NO: 130) | |
| (reactive group)-GGGGGGGGGGGGGGGGGG- | G₁₈ (SEQ ID | SEQ ID NO:27 |
| ARAALWQRLTPGEI | NO: 131) | |
| (reactive group)-GGGGGGGGGGGGGGGGG- | G₁₇ (SEQ ID | SEQ ID NO:28 |
| ARAALWQRLTPGEI | NO: 132) | |
| (reactive group)-GGGGGGGGGGGGGGGG- | G₁₆ (SEQ ID | SEQ ID NO:29 |
| ARAALWQRLTPGEI | NO: 133) | |
| (reactive group)-GGGGGGGGGGGGGGG- | G₁₅ (SEQ ID | SEQ ID NO:30 |
| ARAALWQRLTPGEI | NO: 134) | |
| (reactive group)-GGGGGGGGGGGGGG- | G₁₄ (SEQ ID | SEQ ID NO:31 |
| ARAALWQRLTPGEI | NO: 135) | |
| (reactive group)-GGGGGGGGGGGGG-ARAALWQRLTPGEI | G₁₃ (SEQ ID | SEQ ID NO:32 |
| | NO: 136) | |
| (reactive group)-GGGGGGGGGGGG-ARAALWQRLTPGEI | G₁₂ (SEQ ID | SEQ ID NO:33 |
| | NO: 137) | |
| (reactive group)-GGGGGGGGGGG-ARAALWQRLTPGEI | G₁₁ (SEQ ID | SEQ ID NO:34 |
| | NO: 138) | |
| (reactive group)-GGGGGGGGGG-ARAALWQRLTPGEI | G₁₀ (SEQ ID | SEQ ID NO:35 |
| | NO: 139) | |
| (reactive group)-GGGGGGGGG-ARAALWQRLTPGEI | G₉ (SEQ ID | SEQ ID NO:36 |
| | NO: 140) | |
| (reactive group)-GGGGGGGG-ARAALWQRLTPGEI | G₈ (SEQ ID | SEQ ID NO:37 |
| | NO: 141) | |
| (reactive group)-GGGGGGG-ARAALWQRLTPGEI | G₇ (SEQ ID | SEQ ID NO:38 |
| | NO: 142) | |
| (reactive group)-GGGGGG-ARAALWQRLTPGEI | G₆ (SEQ ID | SEQ ID NO:39 |
| | NO: 143) | |
| (reactive group)-GGGGG-ARAALWQRLTPGEI | G₅ (SEQ ID | SEQ ID NO:40 |
| | NO: 144) | |
| (reactive group)-GGGG-ARAALWQRLTPGEI | G₄ (SEQ ID | SEQ ID NO:41 |
| | NO: 145) | |
| (reactive group)-GGG-ARAALWQRLTPGEI | G₃ | SEQ ID NO:42 |
| (reactive group)-OA-OA-OA-OA-OA-OA-OA-OA-OA-OA- | OA₁₀ | SEQ ID NO:43 |
| ARAALWQRLTPGEI | | |
| (reactive group)-OA-OA-OA-OA-OA-OA-OA-OA-OA- | OA₉ | SEQ ID NO:44 |
| ARAALWQRLTPGEI | | |
| (reactive group)-OA-OA-OA-OA-OA-OA-OA-OA- | OA₈ | SEQ ID NO:45 |
| ARAALWQRLTPGEI | | |
| (reactive group)-OA-OA-OA-OA-OA-OA-OA- | OA₇ | SEQ ID NO:46 |
| ARAALWQRLTPGEI | | |
| (reactive group)-OA-OA-OA-OA-OA-OA- | OA₆ | SEQ ID NO:47 |
| ARAALWQRLTPGEI | | |
| (reactive group)-OA-OA-OA-OA-OA-ARAALWQRLTPGEI | OA₅ | SEQ ID NO:48 |
| (reactive group)-OA-OA-OA-OA-ARAALWQRLTPGEI | OA₄ | SEQ ID NO:49 |
| (reactive group)-OA-OA-OA-ARAALWQRLTPGEI | OA₃ | SEQ ID NO:50 |
| (reactive group)-OA-OA-ARAALWQRLTPGEI | OA₂ | SEQ ID NO:51 |
| (reactive group)-OA-ARAALWQRLTPGEI | OA | SEQ ID NO:52 |
| (reactive group)-KKKKKKKKKKKKKKKKKKKK- | K₂₀ (SEQ ID | SEQ ID NO:53 |
| ARAALWQRLTPGEI | NO: 146) | |
| (reactive group)-KKKKKKKKKKKKKKKKKKK- | K₁₉ (SEQ ID | SEQ ID NO:54 |
| ARAALWQRLTPGEI | NO: 147) | |
| (reactive group)-KKKKKKKKKKKKKKKKKK- | K₁₈ (SEQ ID | SEQ ID NO:55 |
| ARAALWQRLTPGEI | NO: 148) | |
| (reactive group)-KKKKKKKKKKKKKKKKK- | K₁₇ (SEQ ID | SEQ ID NO:56 |
| ARAALWQRLTPGEI | NO: 149) | |
| (reactive group)-KKKKKKKKKKKKKKKK- | K₁₆ (SEQ ID | SEQ ID NO:57 |
| ARAALWQRLTPGEI | NO: 150) | |
| (reactive group)-KKKKKKKKKKKKKKK- | K₁₅ (SEQ ID | SEQ ID NO:58 |
| ARAALWQRLTPGEI | NO: 151) | |
| (reactive group)-KKKKKKKKKKKKKK- | K₁₄ (SEQ ID | SEQ ID NO:59 |
| ARAALWQRLTPGEI | NO: 152) | |
| (reactive group)-KKKKKKKKKKKKK-ARAALWQRLTPGEI | K₁₃ (SEQ ID | SEQ ID NO:60 |
| | NO: 153) | |
| (reactive group)-KKKKKKKKKKKK-ARAALWQRLTPGEI | K₁₂ (SEQ ID | SEQ ID NO:61 |
| | NO: 154) | |
| (reactive group)-KKKKKKKKKKK-ARAALWQRLTPGEI | K₁₁ (SEQ ID | SEQ ID NO:62 |
| | NO: 155) | |
| (reactive group)-KKKKKKKKKK-ARAALWQRLTPGEI | K₁₀ (SEQ ID | SEQ ID NO:63 |
| | NO: 156) | |
| (reactive group)-KKKKKKKKK-ARAALWQRLTPGEI | K₉ (SEQ ID | SEQ ID NO:64 |
| | NO: 157) | |
| (reactive group)-KKKKKKKK-ARAALWQRLTPGEI | K₈ (SEQ ID | SEQ ID NO:65 |
| | NO: 158) | |
| (reactive group)-KKKKKKK-ARAALWQRLTPGEI | K₇ (SEQ ID | SEQ ID NO:66 |
| | NO: 159) | |
| (reactive group)-KKKKKK-ARAALWQRLTPGEI | K₆ (SEQ ID | SEQ ID NO:67 |
| | NO: 160) | |
| (reactive group)-KKKKK-ARAALWQRLTPGEI | K₅ (SEQ ID | SEQ ID NO:68 |
| | NO: 161) | |
| (reactive group)-KKKK-ARAALWQRLTPGEI | K₄ (SEQ ID | SEQ ID NO:69 |
| | NO: 162) | |
| (reactive group)-KKK-ARAALWQRLTPGEI | K₃ | SEQ ID NO:70 |

TABLE 2
Modified Thrombopoietin Peptides Containing a Linking Group

| | Linker | |
|---|---|---|
| Sequence | Abbreviation | SEQ ID NO |
| ARAALWQRLTPGEI-GGGGGGGGGGGGGGGGGGGG- | G₂₀ (SEQ ID | SEQ ID NO:71 |
| (reactive group) | NO: 129) | |
| ARAALWQRLTPGEI-GGGGGGGGGGGGGGGGGGG- | G₁₉ (SEQ ID | SEQ ID NO:72 |
| (reactive group) | NO: 130) | |
| ARAALWQRLTPGEI-GGGGGGGGGGGGGGGGGG- | G₁₈ (SEQ ID | SEQ ID NO:73 |
| (reactive group) | NO: 131) | |
| ARAALWQRLTPGEI-GGGGGGGGGGGGGGGGG-(reactive | G₁₇ (SEQ ID | SEQ ID NO:74 |
| group) | NO: 132) | |
| ARAALWQRLTPGEI-GGGGGGGGGGGGGGGG-(reactive | G₁₆ (SEQ ID | SEQ ID NO:75 |
| group) | NO: 133) | |
| ARAALWQRLTPGEI-GGGGGGGGGGGGGGG-(reactive | G₁₅ (SEQ ID | SEQ ID NO:76 |
| group) | NO: 134) | |
| ARAALWQRLTPGEI-GGGGGGGGGGGGGG-(reactive | G₁₄ (SEQ ID | SEQ ID NO:77 |
| group) | NO: 135) | |
| ARAALWQRLTPGEI-GGGGGGGGGGGGG-(reactive group) | G₁₃ (SEQ ID | SEQ ID NO:78 |
| | NO: 136) | |
| ARAALWQRLTPGEI-GGGGGGGGGGGG-(reactive group) | G₁₂ (SEQ ID | SEQ ID NO:79 |
| | NO: 137) | |
| ARAALWQRLTPGEI-GGGGGGGGGGG-(reactive group) | G₁₁ (SEQ ID | SEQ ID NO:80 |
| | NO: 138) | |
| ARAALWQRLTPGEI-GGGGGGGGGG-(reactive group) | G₁₀ (SEQ ID | SEQ ID NO:81 |
| | NO: 139) | |
| ARAALWQRLTPGEI-GGGGGGGGG-(reactive group) | G₉ (SEQ ID | SEQ ID NO:82 |
| | NO: 140) | |
| ARAALWQRLTPGEI-GGGGGGGG-(reactive group) | G₈ (SEQ ID | SEQ ID NO:83 |
| | NO: 141) | |
| ARAALWQRLTPGEI-GGGGGGG-(reactive group) | G₇ (SEQ ID | SEQ ID NO:84 |
| | NO: 142) | |
| ARAALWQRLTPGEI-GGGGGG-(reactive group) | G₆ (SEQ ID | SEQ ID NO:85 |
| | NO: 143) | |
| ARAALWQRLTPGEI-GGGGG-(reactive group) | G₅ (SEQ ID | SEQ ID NO:86 |
| | NO: 144) | |
| ARAALWQRLTPGEI-GGGG-(reactive group) | G₄ (SEQ ID | SEQ ID NO:87 |
| | NO: 145) | |
| ARAALWQRLTPGEI-GGG-(reactive group) | G₃ | SEQ ID NO:88 |
| ARAALWQRLTPGEI-OA-OA-OA-OA-OA-OA-OA-OA-OA- | OA₁₀ | SEQ ID NO:89 |
| OA-(reactive group) | | |
| ARAALWQRLTPGEI-OA-OA-OA-OA-OA-OA-OA-OA-OA- | OA₉ | SEQ ID NO:90 |
| (reactive group) | | |
| ARAALWQRLTPGEI-OA-OA-OA-OA-OA-OA-OA-OA- | OA₈ | SEQ ID NO:91 |
| (reactive group) | | |
| ARAALWQRLTPGEI-OA-OA-OA-OA-OA-OA-OA-(reactive | OA₂ | SEQ ID NO:92 |
| group) | | |
| ARAALWQRLTPGEI-OA-OA-OA-OA-OA-OA-(reactive | OA₆ | SEQ ID NO:93 |
| group) | | |
| ARAALWQRLTPGEI-OA-OA-OA-OA-OA-(reactive group) | OA₅ | SEQ ID NO:94 |
| ARAALWQRLTPGEI-OA-OA-OA-OA-(reactive group) | OA₄ | SEQ ID NO:95 |
| ARAALWQRLTPGEI-OA-OA-OA-(reactive group) | OA₃ | SEQ ID NO:96 |
| ARAALWQRLTPGEI-OA-OA-(reactive group) | OA₂ | SEQ ID NO:97 |
| ARAALWQRLTPGEI-OA-(reactive group) | OA | SEQ ID NO:98 |
| ARAALWQRLTPGEI-KKKKKKKKKKKKKKKKKKKK- | K₂₀ (SEQ ID | SEQ ID NO:99 |
| (reactive group) | NO: 146) | |
| ARAALWQRLTPGEI-KKKKKKKKKKKKKKKKKKK- | K₁₉ (SEQ ID | SEQ ID NO:100 |
| (reactive group) | NO: 147) | |
| ARAALWQRLTPGEI-KKKKKKKKKKKKKKKKKK- | K₁₈ (SEQ ID | SEQ ID NO:101 |
| (reactive group) | NO: 148) | |
| ARAALWQRLTPGEI-KKKKKKKKKKKKKKKKK-(reactive | K₁₇ (SEQ ID | SEQ ID NO:102 |
| group) | NO: 149) | |
| ARAALWQRLTPGEI-KKKKKKKKKKKKKKKK-(reactive | K₁₆ (SEQ ID | SEQ ID NO:103 |
| group) | NO: 150) | |
| ARAALWQRLTPGEI-KKKKKKKKKKKKKKK-(reactive | K₁₅ (SEQ ID | SEQ ID NO:104 |
| group) | NO: 151) | |
| ARAALWQRLTPGEI-KKKKKKKKKKKKKK-(reactive | K₁₄ (SEQ ID | SEQ ID NO:105 |
| group) | NO: 152) | |
| ARAALWQRLTPGEI-KKKKKKKKKKKKK-(reactive group) | K₁₃ (SEQ ID | SEQ ID NO:106 |
| | NO: 153) | |
| ARAALWQRLTPGEI-KKKKKKKKKKKK-(reactive group) | K₁₂ (SEQ ID | SEQ ID NO:107 |
| | NO: 154) | |
| ARAALWQRLTPGEI-KKKKKKKKKKK-(reactive group) | K₁₁ (SEQ ID | SEQ ID NO:108 |
| | NO: 155) | |
| ARAALWQRLTPGEI-KKKKKKKKKK-(reactive group) | K₁₀ (SEQ ID | SEQ ID NO:109 |
| | NO: 156) | |
| ARAALWQRLTPGEI-KKKKKKKKK-(reactive group) | K₉ (SEQ ID | SEQ ID NO:110 |
| | NO: 157) | |
| ARAALWQRLTPGEI-KKKKKKKK-(reactive group) | K₈ (SEQ ID | SEQ ID NO:111 |
| | NO: 158) | |
| ARAALWQRLTPGEI-KKKKKKK-(reactive group) | K₇ (SEQ ID | SEQ ID NO:112 |
| | NO: 159) | |
| ARAALWQRLTPGEI-KKKKKK-(reactive group) | K₆ (SEQ ID | SEQ ID NO:113 |
| | NO: 160) | |
| ARAALWQRLTPGEI-KKKKK-(reactive group) | K₅ (SEQ ID | SEQ ID NO:114 |
| | NO: 161) | |
| ARAALWQRLTPGEI-KKKK-(reactive group) | K₄ (SEQ ID | SEQ ID NO:115 |
| | NO: 162) | |
| ARAALWQRLTPGEI-KKK-(reactive group) | K₃ | SEQ ID NO:116 |

### Example 3

### Examples of Thrombopoietin Peptide Dimers

### ARAALWQRLTPGEI (SEQ ID NO:24)

### IEGPTLRQWLAARA (SEQ ID NO:11)

TABLE 3
Modified Thrombopoietin Peptides Containing a Linking Group

| | Linker | |
|---|---|---|
| Sequence | Abbreviation | SEQ ID NO |
| (reactive group)-GGGGG-IEGPTLRQWLAARA-GGGGGGGG- | (G₅)(SEQ ID | SEQ ID NO:117 |
| ARAALWQRLTPGEI | NO: 144)(G₈) | |
| | (SEQ ID NO: | |
| | 141) | |
| Ac-IEGPTLRQWLAARA-OA-OA-ARAALWQRLTPGEI-K(OA- | (OA₂)(OA) | SEQ ID NO:118 |
| reactive group) | | |
| (reactive group)-OA-IEGPTLRQWLAARA-OA-OA- | (OA)(OA₂) | SEQ ID NO:119 |
| ARAALWQRLTPGEI | | |
| Ac-IEGPTLRQWLAARA-OA-OA-ARAALWQRLTPGEI-K(OA- | (G₈)(SEQ ID | SEQ ID NO:120 |
| reactive group) | NO: 141) | |
| | (OA) | |
| (reactive group)-GGGGG-ARAALWQRLTPGEI-GGGGGGGG- | (G₅)(SEQ ID | SEQ ID NO:121 |
| IEGPTLRQWLAARA | NO: 144)(G₈) | |
| | (SEQ ID NO: | |
| | 141) | |
| (reactive group)-OA-ARAALWQRLTPGEI-OA-OA- | (OA)(OA₂) | SEQ ID NO:122 |
| IEGPTLRQWLAARA | | |
| Ac-ARAALWQRLTPGEI-GGGGGGGG-IEGPTLRQWLAARAI- | (G₈)(SEQ ID | SEQ ID NO:123 |
| K(OA-reactive group) | NO: 141) | |
| | (OA) | |
| Ac-ARAALWQRLTPGEI-OA-OA-IEGPTLRQWLAARA-K(OA- | (OA₂)(OA) | SEQ ID NO:124 |
| reactive group) | | |
| (reactive group)-GGGGG-ARAALWQRLTPGEI-GGGGGGGG- | (G₅)(SEQ ID | SEQ ID NO:125 |
| ARAALWQRLTPGEI | NO: 144)(G₈) | |
| | (SEQ ID NO: | |
| | 141) | |
| (reactive group)-OA-ARAALWQRLTPGEI-OA-OA- | (OA)(OA₂) | SEQ ID NO:126 |
| ARAALWQRLTPGEI | | |
| Ac-ARAALWQRLTPGEI-GGGGGGGG-ARAALWQRLTPGEI- | (G₈)(SEQ ID | SEQ ID NO:127 |
| K(OA-reactive group) | NO: 141) | |
| | (OA) | |
| Ac-ARAALWQRLTPGEI-OA-OA-ARAALWQRLTPGEI-K(OA- | (OA₂)(OA) | SEQ ID NO:128 |
| reactive group) | | |
| (reactive group)-GGGGG-IEGPTLRQWLAARA-GGGGGGGG- | (G₅)(SEQ ID | SEQ ID NO:168 |
| IEGPTLRQWLAARA | NO: 144)(G₈) | |
| | (SEQ ID NO: | |
| | 141) | |
| IEGPTLRQWLAARA-GGGGGGGG-IEGPTLRQWLAARA-OA-OA- | (Gly₈)(SEQ | SEQ ID NO:169 |
| K-(reactive group) | ID NO: 141) | |
| | (OA₂) | |
| IEGPTLRQWLAARA-GGGGGGGG-IEGPTLRQWLAARA-K-OA- | (Gly₈)(SEQ | SEQ ID NO:170 |
| OA-(reactive group) | ID NO: 141) - | |
| | (K)(OA₂) | |
| (reactive group)-OA-OA-IEGPTLRQWLAARA-OA-OA-OA- | (OA₂)(OA₃) | SEQ ID NO:171 |
| IEGPTLRQWLAARA | | |
| IEGPTLRQWLAARA-GGG-K[OA-OA-(reactive group)]GGGG- | (Gly₃)- | SEQ ID NO:172 |
| IEGPTLRQWLAARA | (K)(OA₂) | |
| | (Gly₄)(SEQ | |
| | ID NO: 205 | |
| (reactive group)-GGGGG-LQGPTLRAWRAGMA-GGGGGGGG- | (G₅)(SEQ ID | SEQ ID NO:184 |
| IEGPTLRQWLAARA | NO: 144)(G₈) | |
| | (SEQ ID NO: | |
| | 141) | |
| (reactive group)-GGGGG-IEGPTLRQWLAARA-GGGGGGGG- | (G₅)(SEQ ID | SEQ ID NO:185 |
| LQGPTLRAWRAGMA | NO: 144)(G₈) | |
| | (SEQ ID NO: | |
| | 141) | |
| LQGPTLRAWRAGMA-GGGGGGGG-IEGPTLRQWLAARA- | (G₈)(SEQ ID | SEQ ID NO:186 |
| GGGGGK-(reactive group) | NO: 141)(G₅) | |
| | (SEQ ID NO: | |
| | 144) | |
| IEGPTLRQWLAARA-GGGGGGG-IEGPTLRQWLAARA-GGGGGK- | (G₈)(SEQ ID | SEQ ID NO:187 |
| K-(reactive group) | NO: 141)(G₅) | |
| | (SEQ ID NO: | |
| | 144) | |
| (reactive group)-GGGGG-LQGPTLRAWRAGMA-GGGGGGGG- | (G₅)(SEQ ID | SEQ ID NO:188 |
| LQGPTLRAWRAGMA | NO: 144)(G₈) | |
| | (SEQ ID NO: | |
| | 141) | |
| (reactive group)-GGGGG-LQGCTLRAWRAGMC (cyclic | (G₅)(SEQ ID | SEQ ID NO:189 |
| peptide) | NO: 144) | |
| IEGPTLRQWLAARA-GGGGGGGG-IEGPTLRQWLAARA-(OA)-K- | (G₈)(SEQ ID | SEQ ID NO:190 |
| (reactive group) | NO: 141) | |
| | (OA)(K) | |
| LQGPTLRAWRAGMA-GGGGGGGG-IEGPTLRQWLAARA-(OA)-K- | (G₈)(SEQ ID | SEQ ID NO:191 |
| (reactive group) | NO: 141) | |
| | (OA)(K) | |

In the above table, “reactive group” designates a reactive group as described herein, e.g., a succinimide-containing reactive group or a maleimide-containing reactive group (such as NHS, sulfo-NHS, MBS, GMBS, GMBS and/or MPA). “OA” designates 8-amino octanoic acid, as described in the sections above. “K(OA-reactive group)” “K[OA-OA-(reactive group)]” refers to a lysine residue wherein a reactive group is linked to the epsilon amino group of the lysine side chain via one or more 8-amino octanoic acids (in this case, one OA or two OA monomers). In the above table, all modified peptides have an amide (CONH₂) at their C-termini.

### Example 4

### In Vivo Potency of Thrombopoietin Peptides and Conjugates

This Example shows the results of in vivo testing of selected modified TPO peptides and albumin conjugates thereof.

Ten to twelve-week old female CD-1 mice were administered with a single injection of the TPO derivatives indicated below at 10 nmol/kg (n=4/group). The DAC™ compounds in Table 4 are variants of TPO peptides modified to include an MPA reactive group, thereby forming an MPA-modified unconjugated peptides. The DAC™ compounds in Table 4 were administered intravenously. Albumin conjugates of the DAC™ compounds (also referred to herein as Preformed Conjugates (PC)) were injected subcutaneously. For each mouse, a blood sample was collected prior to dosing and 6 days postdose via the mandibular artery. Platelet counts were measured using an ABC Vet analyzer using standard procedures.

Platelet count fold increases are reported in Table 4 and 5 for MPA-modified TPO peptide and the albumin TPO conjugate, respectively.

TABLE 4
Platelet count increase following a single intravenous
injection of MPA-modified TPO peptide

| MPA-Modified | Platelet Count |
|---|---|
| TPO Peptide | Fold Increase |
| MPA-modified form of | 5.1 |
| SEQ ID NO: 168 | |
| (SEQ ID NO: 173) | |
| MPA-modified form of | <3 |
| SEQ ID NO: 117 | |
| (SEQ ID NO: 174) | |
| MPA-modified form of | <3 |
| SEQ ID NO: 118 | |
| (SEQ ID NO: 175) | |
| MPA-modified form of | <3 |
| SEQ ID NO: 121 | |
| (SEQ ID NO: 176) | |
| MPA-modified form of | <3 |
| SEQ ID NO: 122 | |
| (SEQ ID NO: 177) | |
| MPA-modified form of | <3 |
| SEQ ID NO: 123 | |
| (SEQ ID NO: 178) | |
| MPA-modified form of | <3 |
| SEQ ID NO: 124 | |
| (SEQ ID NO: 179) | |
| MPA-modified form of | <3 |
| SEQ ID NO: 125 | |
| (SEQ ID NO: 180) | |
| MPA-modified form of | <3 |
| SEQ ID NO: 126 | |
| (SEQ ID NO: 181) | |
| MPA-modified form of | <3 |
| SEQ ID NO: 127 | |
| (SEQ ID NO: 182) | |
| MPA-modified form of | <3 |
| SEQ ID NO: 128 | |
| (SEQ ID NO: 183) | |
| MPA-modified form of | <3 |
| SEQ ID NO: 184 | |
| (SEQ ID NO: 192) | |
| MPA-modified form of | <3 |
| SEQ ID NO: 185 | |
| (SEQ ID NO: 193) | |
| MPA-modified form of | 5.0 |
| SEQ ID NO: 190 | |
| (SEQ ID NO: 194) | |
| MPA-modified form of | <3 |
| SEQ ID NO: 191 | |
| (SEQ ID NO: 195) | |
| MPA-modified form of | 4.4 |
| SEQ ID NO: 169 | |
| (SEQ ID NO: 196) | |
| MPA-modified form of | 4.7 |
| SEQ ID NO: 170 | |
| (SEQ ID NO: 197) | |
| MPA-modified form of | 3.0 |
| SEQ ID NO: 171 | |
| (SEQ ID NO: 198) | |
| MPA-modified form of | 5.0 |
| SEQ ID NO: 172 | |
| (SEQ ID NO: 199) | |

TABLE 5
Platelet count increase following a single subcutaneous
injection of albumin-conjugated TPO

| Albumin-TPO | Platelet Count |
|---|---|
| Conjugate | Fold Increase |
| Albumin-conjugate of | 4.3 |
| SEQ ID NO: 168 | |
| (SEQ ID NO: 200) | |
| Albumin-conjugate of | 3.8 |
| SEQ ID NO: 169 | |
| (SEQ ID NO: 201) | |
| Albumin-conjugate of | 4.4 |
| SEQ ID NO: 170 | |
| (SEQ ID NO: 202) | |
| Albumin-conjugate of | 2.0 |
| SEQ ID NO: 171 | |
| (SEQ ID NO: 203) | |
| Albumin-conjugate of | 4.7 |
| SEQ ID NO: 172 | |
| (SEQ ID NO: 204) | |

### Example 5

### Solubilization of MPA-Modified TPO Peptides

MPA-modified TPO peptide of SEQ ID NO:171 is relatively insoluble in aqueous solution. In order to increase the solubility of this peptide, 10 mg of lyophilized MPA-modified TPO peptide of SEQ ID NO:171 was placed into a 100 μl volume of either 50% methanol/water, 50% ethanol/water, or 50% DMSO/water to make a stock solution of 25 mM. The sample was then diluted further to 19 mM using water only.

Solubilized MPA-modified TPO peptide of SEQ ID NO:171 (19 mM) was then diluted 1/10 slowly (dropwise) into a 20% (w/v) (3 mM) solution of recombinant human albumin, to make a final concentration of 1.9 mM (final molar ratio of modified peptide to rHA=0.7).

The mixture of the modified peptide and rHA was then agitated at room temperature for at least 30 min to allow for complete conjugation.

### Example 6

### Purification of TPO-albumin Conjugates

TPO-albumin conjugates were equilibrated in loading buffer: 20 mM sodium phosphate (pH 7), 5 mM sodium octanoate, 750 mM ammonium sulfate prior to loading onto Butyl-Sepharose Fast-Flow 4 resin (GE Healthcare) (50 ml bed volume) pre-equilibrated in loading buffer. The flow-rate of mobile phase was maintained at 1 ml/min. Under these conditions, free albumin elutes from the column and all TPO-albumin conjugates remained bound to the resin. TPO-albumin conjugates were then eluted from the column by applying a linear gradient of decreasing ammonium sulfate concentration over 5 column volumes. TPO-albumin conjugates eluted at 50-300 mM ammonium sulfate, and free unconjugated-TPO eluted within the water wash following completion of the ammonium sulfate gradient.

Complete removal of unbound unconjugated-TPO from the TPO-albumin conjugates was achieved by adding a second hydrophobic interaction chromatographic step either prior to or following the aforementioned method using Butyl-Sepharose resin. For example, a flow-thru column composed of Phenyl-Sepharose resin (GE Healthcare) equilibrated in 20 mM sodium phosphate (pH 7), 5 mM sodium octanoate, 5 mM ammonium sulfate provides complete removal of unbound unconjugated-TPO from TPO-albumin conjugates. Under these conditions, only unbound unconjugated-TPO binds to the resin.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A modified thrombopoietin peptide wherein the peptide is modified by the covalent attachment of a reactive group to the peptide, wherein the covalent attachment is optionally through a linking group, and wherein the reactive group is capable of forming a covalent bond with an amino, carboxyl, hydroxyl, or thiol group of a serum protein.

2. A conjugate comprising a modified thrombopoietin peptide, wherein the peptide is modified by the covalent attachment of a residue of a reactive group, optionally through a linking group, to the peptide, and wherein the residue of the reactive group is covalently attached to a serum protein.

3. The modified peptide of claim 1 or the conjugate of claim 2 where the amino acid sequence of the thrombopoietin peptide comprises SEQ ID NO:1:
X¹ X² X³ X⁴ X⁵ X⁶ X⁷
where X¹ is C, L, M, P, Q or V; X² is F, K, L, N, Q, R, S, T or V; X³ is C, F, I, L, M, R, S, V or W; X⁴ is any of the 20 genetically coded L-amino acids; X⁵ is A, D, E, G, K, M, Q, R, S, T, V or Y; X⁶ is C, F, G, L, M, S, V, W or Y; and X⁷ is C, G, I, K, L, M, N, R or V.

4. The modified peptide of claim 1 or the conjugate of claim 2 where the amino acid sequence of the thrombopoietin peptide comprises a sequence selected from the group consisting of:
(SEQ ID NO:4)
G G C A D G P T L R E W I S F C G G;
(SEQ ID NO:5)
G N A D G P T L R Q W L E G R R P K N;
(SEQ ID NO:6)
G G C A D G P T L R E W I S F C G G K;
(SEQ ID NO:7)
T I K G P T L R Q W L K S R E H T S;
(SEQ ID NO:8)
S I E G P T L R E W L T S R T P H S;
(SEQ ID NO:9)
L A I E G P T L R Q W L H G N G R D T;
(SEQ ID NO:10)
C A D G P T L R E W I S F C;
(SEQ ID NO:11)
I E G P T L R Q W L A A R A;
(SEQ ID NO:206)
L Q G P T L R A W R A G M A;
and
(SEQ ID NO:207)
L Q G C T L R A W R A G M C.
or a derivative thereof.

5. The modified peptide of claim 1 or the conjugate of claim 2, wherein the covalent attachment of the reactive group is at substantially only one site on the peptide.

6. The conjugate of claim 5, wherein the covalent attachment of the reactive group is at substantially only one site on the peptide and wherein the residue of the reactive group is covalently attached to the serum protein at substantially only one site on the protein.

7. The conjugate of claim 6, wherein the covalent attachment of the reactive group is at the epsilon amino group of a lysine in the peptide, optionally through a linking group, and wherein the residue of the reactive group is covalently attached to albumin through a maleimide group to the thiol group of cysteine 34 of albumin.

8. The conjugate of claim 7, wherein the maleimide group is part of a maleimido propionic acid (MPA).

9. The modified peptide of claim 1 or the conjugate of claim 2, wherein the serum protein is selected from the group consisting of albumin, transferrin, ferritin, IgM, IgG, a steroid binding protein, a thyroxin binding protein, and alpha-2-macroglobulin.

10. The modified peptide or the conjugate of claim 9, wherein the serum protein is albumin.

11. The modified peptide or conjugate of claim 10, wherein the albumin is recombinant.

12. The modified peptide or conjugate of claim 10, wherein the albumin is human.

13. The modified peptide of claim 1 or conjugate of claim 2, wherein the reactive group is a maleimide containing group.

14. The modified peptide or conjugate of claim 13, wherein the reactive group is gamma-maleimide-butylamide (GMBA) or maleimido propionic acid (MPA).

15. The modified peptide or conjugate of any one of claims 1-14, wherein the peptide is covalently attached to the reactive group through one or more linking groups.

16. The modified peptide or conjugate of claim 15, wherein the one or more linking groups are each independently selected from the group consisting of polyglycine, polylysine, AEA ((2-amino) ethoxy acetic acid), AEEA ([2-(2-amino)ethoxy)]ethoxy acetic acid), and OA (8-amino octanoic acid).

17. The modified peptide or conjugate of claim 15, wherein the one or more linking groups are (AEEA)ₙ and where n is 0, 1, 2, 3, 4, 5, or 6.

18. The modified peptide or conjugate of claim 15, wherein the one or more linking groups are (OA)ₙ and where n is 0, 1, 2, 3, 4, 5, or 6.

19. The modified peptide or conjugate of claim 15, wherein the peptide or conjugate comprises a formula selected from the group consisting of:
Reactive Group-(Gly₅)-TMP₁-(Gly₈)-TMP₂ (SEQ ID NO: 164) (Formula Va);
Protein-X-Residue-(Gly₅)-TMP₁-(Gly₈)-TMP₂ (SEQ ID NO: 164) (Formula Vb);
TMP₁-(Gly₈)-TMP₂-(OA₂)-K-reactive group (SEQ ID NO: 165) (Formula VIa);
TMP₁-(Gly₈)-TMP₂-(OA₂)-K-residue-X-protein (SEQ ID NO: 165), (Formula VIb);
TMP₁-(Gly₈)-TMP₂-K-(OA₂)-reactive group (SEQ ID NO: 166) (Formula VIb);
TMP₁-(Gly₈)-TMP₂-K-(OA₂)-residue-X-protein (SEQ ID NO: 166) (Formula VIb);
Reactive group-(OA₂)-(TMP)₁-(OA₃)-(TMP₂) (Formula VIIIa);
Protein-X-residue-(OA₂)-(TMP)₁-(OA₃)-(TMP₂) (Formula VIIb); and 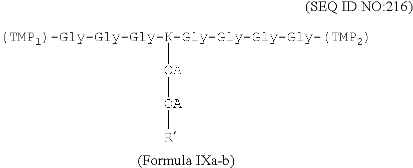
wherein
TMP₁ and TMP₂ are each a peptide having a sequence of X¹ X² X³ X⁴ X⁵ X⁶ X⁷ where X¹ is C, L, M, P, Q or V; X² is F, K, L, N, Q, R, S, T or V; X³ is C, F, I, L, M, R, S, V or W; X⁴ is any of the 20 genetically coded L-amino acids; X⁵ is A, D, E, G, K, M, Q, R, S, T, V or Y; X⁶ is C, F, G, L, M, S, V, W or Y; and X⁷ is C, G, I, K, L, M, N, R or V. In one embodiment, TMP₁ and TMP₂ are each a peptide having a sequence IEGPTLRQWLAARA (SEQ ID NO:11);
OA is an 8-aminooctanoyl linking group;
R′ is either a reactive group in Formula IXa, or a residue of a reactive group following conjugation in Formula IXb;
protein is a serum protein and X is a —S—, —N(H)— or —O— from said protein, e.g. from an amino acid side chain;
Gly or G is glycine; and
K is lysine and the reactive group is linked to the lysine through the lysine's epsilon amino group to form the reactive group residue.

20. The modified peptide or conjugate of any one of claims 1 or 2, wherein the peptide is selected from the group consisting of SEQ ID NOS 1-128, 168-204, and 206-209, or a derivative thereof.

21. The modified peptide or conjugate of claim 19, wherein TMP₁ and TMP₂ are each a peptide having a sequence IEGPTLRQWLAARA (SEQ ID NO:11).

22. The conjugate as claimed in claim 2, wherein the conjugate has a decreased rate of excretion and/or an increased circulating half-life in vivo compared to a corresponding unconjugated peptide.

23. The conjugate of claim 22, wherein the conjugate retains at least some of the biological activity of the unconjugated peptide.

24. The conjugate of claim 22, wherein the conjugate exhibits more biological activity than the unconjugated peptide.

25. The conjugate of claim 23 or 24, wherein the biological activity is the production of megakaryocytes or platelets or both.

26. The conjugate of claim 23 or 24, wherein the biological activity is the ability to bind to a thrombopoietin receptor.

27. The modified peptide of claim 1 or conjugate of claim 2 wherein the peptide is N-terminal acylated and/or C-terminal amidated.

28. A method for extending the in vivo half-life of a modified peptide as claimed in any one of claims 1, 3-5 and 9-21, the method comprising contacting the modified peptide with a serum protein under conditions suitable for the formation of a covalent bond between the reactive group and the serum protein, thereby forming a modified peptide conjugated to the serum protein, which modified peptide conjugated to the serum protein has an increased in vivo half-life in the blood relative to the unconjugated modified peptide.

29. The method of claim 28, wherein the contacting is performed in vivo.

30. The method of claim 28, wherein the contacting is performed in vitro.

31. The method of claim 28, wherein the serum protein is albumin.

32. The method of claim 31, wherein the serum protein is recombinant human albumin.

33. A method for treating or preventing a disease or disorder treatable with a thrombopoietin peptide in a subject, the method comprising administering to the subject an amount of the modified peptide or conjugate of any one of claims 1, 2, 19 or 21, effective for said treating or preventing.

34. The method of claim 33, wherein the disease or disorder is thrombocytopenia.

35. The method of claim 34, wherein the thrombocytopenia is caused by cancer therapy.

36. The method of claim 34, wherein the thrombocytopenia is immune thrombocytopenic purpura.

37. A method for harvesting platelets in a subject, the method comprising administering to the subject an amount of the modified peptide or conjugate of any one of claims 1, 2, 19 or 21, and harvesting platelets from the subject.

38. A method for increasing platelets in a subject, the method comprising administering to the subject an amount of the modified peptide or conjugate of any one of claims 1, 2, 19 or 21.

39. The method of claim 33, wherein the subject is human.

40. A pharmaceutical composition comprising the modified peptide or conjugate of any one of claims 1 or 2, and a pharmaceutically acceptable carrier, excipient, or diluent.

41. A kit comprising in one or more containers the modified peptide or conjugate of any one of claims 1 or 2.

42. The kit of claim 41, wherein the modified peptide or conjugate is in lyophilized form.

43. The kit of claim 42, further comprising a container of sterile solution suitable for reconstituting the lyophilized modified peptide or conjugate.

44. The kit of claim 41, wherein the modified peptide or conjugate is in liquid form.

45. A syringe containing in solution the modified peptide or conjugate of any of claims 1, 2, 19 or 21.

46. The modified peptide of claim 1 or the conjugate of claim 2 wherein the peptide has a CONH₂ at its carboxy terminus.

47. The modified peptide or conjugate of claim 3 or 9, wherein the peptide has a CONH₂ at its carboxy terminus.

48. The conjugate of claim 6, wherein the peptide has a CONH₂ at its carboxy terminus.

49. The method of claim 28, wherein the peptide has a CONH₂ at its carboxy terminus.

50. The pharmaceutical composition of claim 40, wherein the peptide has a CONH₂ at its carboxy terminus.

51. The kit of claim 41, wherein the peptide has a CONH₂ at its carboxy terminus.

52. The syringe of claim 45 wherein the peptide has a CONH₂ at its carboxy terminus.

53. A method of increasing the solubility of a modified thrombopoietin peptide, comprising contacting the modified peptide with an aqueous solution comprising a solubilizing agent, wherein the peptide is modified by the covalent attachment of a reactive group to the peptide, wherein the covalent attachment is optionally through a linking group, and wherein the reactive group is capable of forming a covalent bond with an amino, carboxyl, hydroxyl, or thiol group of a serum protein; and wherein the solubilizing agent is chosen from an alcohol or a polar aprotic solvent.

54. The method of claim 53, wherein the alcohol is selected from the group consisting of ethanol and methanol.

55. The method of claim 53, wherein the polar aprotic solvent is dimethyl sulfoxide.

56. A method of purifying a conjugated modified thrombopoietin peptide from unconjugated forms thereof, comprising:
(i) obtaining a sample containing a mixture of conjugated and unconjugated forms of the modified thrombopoietin peptide;
(ii) contacting the sample with a first hydrophobic support under conditions such that the conjugated forms of the modified thrombopoietin peptide are substantially bound to the support, whereas the unconjugated forms are substantially unbound;
(iii) (optionally) removing the unconjugated forms of the modified thrombopoietin peptide from the support;
(iv) (optionally) prior to or following step (ii) contacting the sample with a second hydrophobic support under conditions such that the conjugated forms of the modified thrombopoietin peptide are substantially bound to the support, whereas the unconjugated forms are substantially unbound;
(v) eluting the conjugated modified thrombopoietin peptide from the support, wherein the peptide is modified by the covalent attachment of a reactive group to the peptide, wherein the covalent attachment is optionally through a linking group, and wherein the reactive group is capable of forming a covalent bond with an amino, carboxyl, hydroxyl, or thiol group of a serum protein.

57. The method of claim 56, wherein the first and second hydrophobic supports are the same.

58. The method of claim 56, wherein the first and second hydrophobic supports are different.

59. The method of claim 58, wherein the first hydrophobic support is a butyl sepharose resin.

60. The method of claim 58, wherein the second hydrophobic support is a phenyl sepharose resin.
